SEQUENCE LISTING (95 sequences)

SEQ ID NO: 1 - DNA, 1605 bp, Yarrowia lipolytica
  misc_feature: YALI0B22308g
atggttcatcttggtccccgaaaacccccgtcccgaaagggctcaatggcagacgtcccgcgggacctgctggagcaaatctcccagcttgaaaccatcttcaccgtttcgcccgaaaagctgcgtcaaatcaccgaccactttgtgtccgagctcgctaaaggcctcacaaaggagggtggagatatccccatgaaccccacctggattctgggatggcccaccggaaaggagagcggctgctatctggctctcgacatgggtggcaccaacctgcgagttgtcaaggtgactctggacggcgaccgaggcttcgacgtcatgcagtccaagtaccacatgccccccaacatcaaggtcggcaagcaagaggagctgtgggagtacattgccgaatgtctgggcaagttcttggccgacaattatcctgaggctcttgatgcccatgagcgaggacgagatgtcgacagaaccgctgcgcagagcttcactcgagacaagtctcctcctccccacaaccagcacatttcgtgttctcctggcttcgacatccacaagattcctctcggtttcaccttttcatatccctgctctcagcccgccgtcaaccgaggtgtactgcagcgatggaccaagggtttcgacattgagggagtcgagggcgaggacgtggtccccatgctggaagctgccctcgaaagaaagaacattcctatttccatcaccgccctgatcaacgacaccaccggaactatggtggcctccaactaccacgacccccagatcaagctgggtaacatctttggtactggtgtcaacgccgcctactacgagaaggtcaaggacattcccaagctcaagggtctcatccccgacagcattgatcccgagacccccatggccgtcaattgcgagtatggagccttcgacaatgagcacaaggttctccctagaaccaagtgggacatcatcatcgatgaggagtctccccgacccggtcagcagaccttcgagaagatgagtgctggctactacctgggagaattgcttcgtctggttcttctggacctgtacaaggacgggtttgtgttcgagaaccagggcaagaacggtcaggagcttggaaacggcaacatcaacaagtcgtatttcttcgacacctctttcctgtctctgattgaggaggatccctgggagaacttgactgatgtcgagattctcttcaaggagaagcttggtattaacaccactgagcccgagcgaaagctcattcgtcgactggccgagctcattggtactcgatccgctcgaatctctgcctgtggtgtcgctgccatctgtaagaaggctggctacaaggaggctcacgctggagctgacggatccgtgttcaacaagtaccccggattcaaggagcgaggcgcccaggctctcaacgagatttttgagtggaacctgcccaaccctaaggaccaccccatcaaaatcgttcccgctgaggatggtagcggtgttggagctgctctgtgcgctgctctcaccatcaagcgagtcaagcagggtcttcccgttggtgtcaagcccggtgtcaagtacgatatttag

SEQ ID NO: 2 - protein, 534 aa, Yarrowia lipolytica
  misc_feature: YALI0B22308p
MVHLGPRKPPSRKGSMADVPRDLLEQISQLETIFTVSPEKLRQITDHFVSELAKGLTKEGGDIPMNPTWILGWPTGKESGCYLALDMGGTNLRVVKVTLDGDRGFDVMQSKYHMPPNIKVGKQEELWEYIAECLGKFLADNYPEALDAHERGRDVDRTAAQSFTRDKSPPPHNQHISCSPGFDIHKIPLGFTFSYPCSQPAVNRGVLQRWTKGFDIEGVEGEDVVPMLEAALERKNIPISITALINDTTGTMVASNYHDPQIKLGNIFGTGVNAAYYEKVKDIPKLKGLIPDSIDPETPMAVNCEYGAFDNEHKVLPRTKWDIIIDEESPRPGQQTFEKMSAGYYLGELLRLVLLDLYKDGFVFENQGKNGQELGNGNINKSYFFDTSFLSLIEEDPWENLTDVEILFKEKLGINTTEPERKLIRRLAELIGTRSARISACGVAAICKKAGYKEAHAGADGSVFNKYPGFKERGAQALNEIFEWNLPNPKDHPIKIVPAEDGSGVGAALCAALTIKRVKQGLPVGVKPGVKYDI

SEQ ID NO: 3 - protein, 533 aa, Yarrowia lipolytica
  misc_feature: Yarrowia lipolytica putative sugar transporterYSP1
MFWKNMKNEPRQVLNSTLWLSVIVFGLLGSARGLDEGMIAGTTSQASFEHQFNLKDPTKTANQQANELSNITAMVQIGSVGGALIAMFVQDRIGRIRCLQEMIILWTVGVIIEVTSYSQGQLLAGRFVAGLGIGQSVVVGPTYLAEVAPKNVRGLCTCIFSGSVYLGVMLEYFANYSTTLHMSPNSRIQWVLPTAVQFIFAGLLFIGSFFIKESPRWLMKIGKDELAVETLSKIRHLPVDDLYVQGEIVDVREQIEREKQALSGTSILSLLKELVSTKANRYRLFLGIMVQLLGQWSGANAVTVYSPKFFSMLGIPSKTDQMMYTAVLGVIKFTSAICCALFLIDTIGRRRSLYTGICLQFVSMLYLGIYLAIVPATVGVDRSPSQKKAGGAAIAAIYLSGCGWALGWNSIQYLINAEIYTVRHRSLASGIIMVFHFANQYGNSKALPFMRSGITDHGSMFFFAGVLLLGLAWSWFFLPEVSGRSLESIDEMFSLPWYQIGRRGHKLVPETGTVIQIQEEEEKKGGVIHVENC

SEQ ID NO: 4 - protein, 575 aa, Yarrowia lipolytica
  misc_feature: Yarrowia lipolytica putative sugar transporterYSP2
MKLFKREAPQGTVRTSYFGSRGTRLHRIIAAVAGIGFLLFGYDTGVMGSLLTLPTFIHQFPTMDSTSPHLDAKTKSFHSTVQGTAVAIYEIGCMLGALFTMWGGDKLGRRKIIFIGSIIVTIGAILQCASYSLGQFVTGRVIAGIGVGFTTATVPMFQAECARPERRGALVMLGGALTTGGIALSYWIDFGFYFVKRNDSDWRFPVAFQILFSLILSCTVLYLPESPRWLIKKGRYEEAAGVFAALEDVPIDDIYVSQQLMQVKESLMVGQLAQEGIEGDEARRRIASGDVELGEEPPFRKQLVLLFTFGKKKHLHRAMLAYSQQIMHQMCGINLISYYAAYIFQTSIGMSPLNSRILAACDGTEYFLASWIAFYTIERFGRRKLMLFGTIGQACTMAILAGTVYAASSVKDGGLDKPQAGIAAAVFLFVFNTFFSIGWLGMAWLYPAEIAPIEIRAISQGLSTSGNWVFNFLVVMITPVAFNSIKWRTYIIFACINVAMVPSIYFFFPETMGRSLEEIDLIFEDSNPRTPWDVVGIAKRLPRGTLDGVVPSDMELEKEIEHVSVKSLGGATIRG

SEQ ID NO: 5 - protein, 505 aa, Yarrowia lipolytica
  misc_feature: Yarrowia lipolytica putative sugar transporterYSP3
MSIKSLSKGSSLDDTCVGISRVATPTNLEKDLSLWETMKIYKKATFWSFVMCCTIIMDGYDGNMVPSFYALPVFQKKFGIQLPNGDWTVEAKWQTAFLVGVPIGRITGAVGVGLLADRFGRKKVTITSLAFMVGVTFVVFFATGKEMLCAGWIISGIIWGVFNTMAPTYVSEVCPIKMRSLLAAAINLSWVAGQFISTGVVTGTSTRTDDWAYRVPLAVQWVFPAILIPTLCFAPESPWWLLRRGEIDKARNALARLADETKIDLDAHLEHMIQTDKEEDKSGTIAECFKGPDLHRTEICSMVYSIQPLSGANIINFFAFFFQLTGVPQDIIFKLTLGLTGLGFLATLLSSVPIARVGRRKIMLTGLFVLTVALFAMGILGCFTSRGTNWATAILLFVWVGTYDLSIGPGAFVIYSESSSVRLRSKTIAIASVISSTVTLVFNVAVPYMLNEAEANMRGLVGFVYGPLCILSLIWVYFRLPELKGLSYMEIDRLFEGKKVAKDGV

SEQ ID NO: 6 - protein, 579 aa, Yarrowia lipolytica
  misc_feature: Yarrowia lipolytica putative sugar transporterYSP4
MSHRPWDCLPLWNTVVVFRRCVLWSLVMCFVIVMDGYDGFLIPSFYAVPSFQRRYGVQLPDGSWTVEAKWQTAYMVGAPIGRIAGALGVGVLADRFGRKRVTLVGLFCLSGITFIVFFAVGKAMLVVGWFLSGLIWGIFNTMTPTYVSEICPVSLRSTFAAAINLSWVVGQFISTAVITASESRQDEWAYRVPLAVQWVFPVVLIPLVMVMPESPWWILKHGDKKGARQVLTRLMDEKDVDIDMYLEYMRQTLEDEVDGGSFVDCFKGSDVRRTEICCLTYFFQPLSGLYILSYAAYFLQLTGIPQNVVFKLTLGLTGLAVMASLVAPIVILTFTRRSIYMGSLALMAATLFAIGITACYDTQAAKWAAPVLIYVWVGTYDATIGPLTYVIVSETSSVKLRSKTIALASITNSIMVLVLHISVPYMMNEEEANMDGFVGFIYAPFCLLAIAWAWWRLPELKGMSFMEIDMLFGNETGTQRAPLVFDGQKRERQPLESLDGLGWRFGGQQDLSETNDNNEGSREERLSNPYCSSNEDNRSDRFQPSERGCILDSPKRKHAIEKNGVVITETLEMGENADR

SEQ ID NO: 7 - protein, 554 aa, Yarrowia lipolytica
  misc_feature: Yarrowia lipolytica hexose transporterYHT5, strain E150
MYKVHNPYLTAAVATMGGMLFGFDISSVSAFVGEDNYMNYFGHPTSFQQGGITASMAGGSMLSCAFAGYISDRVGRKPTIQFAAAWWMVGASIQCSAQNMGQLIAGRAISGLGIGLGSSQIPVFISELSPKKIRGRLVGCFQWSVTWGILIMFYISFGCSYIKGHSSFRLAWGIQLIPGAMLAFGMMLLDESPRWLASKDRWEEAIQIIRSINANYGSEEDILMEIEDLREVVRIDHESKSVTIWDLFRKDSINRTMVGVWAQIWQQLTGMNIMMYYVVIIFKMAGYSGKSAVIVSGSIQYIINVVMTIPALLFIDKIGRRPLLLCGSMLMATWLLAVGGMLGAYGIQMPQGLPAVPSKNQAADPYTTIYIPDNQAPARKAIIACCYLFVASFAPTWGPGIWLYCSEIFPNKQRALANSLTAGANWGFNFALALFVPTAFKNINWKVYIIFGVFCIVMSIHVFLLFPETKGKSLEVIDQMWDARVPAWKTASWVPDHMPSHYAGDQEEKPTDELAEAPFHEENAPVNTETPPHEDEPTFAETEPKTQYPGTEHV

SEQ ID NO: 8 - protein, 554 aa, Yarrowia lipolytica; identical to SEQ ID NO: 7
  misc_feature: Yarrowia lipolytica hexose transporterYHT5 - strain H222

SEQ ID NO: 9 - protein, 554 aa, Yarrowia lipolytica; identical to SEQ ID NO: 7
  misc_feature: Yarrowia lipolytica hexose transporterYHT5; strain W29

SEQ ID NO: 10 - protein, 545 aa, Yarrowia lipolytica
  misc_feature: Yarrowia lipolytica YHT6
MIGNAQINQVGALQHRFPKLHNPYLTAAVATMGGLLFGFDISSVSAFVDTKPYKEYFGYPTSIQQGGITASMAGGSFLSSLVAGWISDRLGRRFAIHFASFWWVVGAAIQSSAQNKGQLIAGRLISGLGIGLGSSVIPVYISELSPKKIRGRLVGLFQWAVTWGILIMFYISFGLSNIHGVAGFRVAWGLQIIPGLLMSLGCLFLEESPRWLAKQDNWDESVRVLRAIHQGGYGTEEDILLEIEEIREAVRIEHETKNLRFWHLFQKDSINRTMVGIWAQIWQQLTGMNVMMYYIVLIFTMAGYTGNANLVASSIQYVINMIMTIPALLFIDRVGRRPLLLFGSIVMMIWLFAVAGILAVYGTQIPGGLDGDAFTTIVIEPTHKPAQKGVIACSYLFVATFAPTWGPGIWLYCSELFPLKQRAVAAGVTASANWIFNFALALFVPSAFKNINWKTYIIFGVFCIVMTIHVFVLFPETKGKTLEEIDMMWAARVPAWRTANWVPDHVPGALPEDEKHSEEMVEAVESNEEEPKIASANVDAPPSQL

SEQ ID NO: 11 - protein, 582 aa, Yarrowia lipolytica
  misc_feature: Yarrowia lipolytica putative sugar transporterYSP7
MKDFLAFTKASTEVKTVFFGCRGTPLHRVVAAIAGIGFLLFGYDQGVMGGLLTLPTFIQEFPSMDTSDHLPPDVKTHNTTIQGTAVGIYEIGCMLGALFTMWAGDKLGRRYMIFWGSIIITIGAILQCAAYSLAQFIVGRVIAGIGNGFITATVPMLQAECARPERRGALVMLEGALVTGGIALSYWIDFGFYFVKTNDADWRFPVAFQCVFSMFLTFTVMSLPESPRWLVKKGRYEEAAGVFAALEDVALDDPYVIDQVTRVKESIIMGQLAQHGIEGEEAQRKMAAGECQMGDELPFWQQIKLLFTFGKKKHFHRTMLAYWIQVMHQVSGINLITYYAAYIYQTSIGMNAMNSRILAACNGTEYFLASWVAFYTIERFGRRKLMLFGTIGQACTMAILTGCVYASSTPADGGLGNEGAGIAAAVFLFVFNSFFAIGWLGMSWLYPAEITSLEVRAPANGLATSGNWVFNFMVVMVTPVAFNSIKWRTYIIFACINAAMIPTIYFMYPETMGKSLEDIDMIFALSNPKTPWDVVGISRRMPSSIDNASPAPIILEKPSQENLEYAPSMSEGTINSVRIETA

SEQ ID NO: 12 - protein, 476 aa, Yarrowia lipolytica
  misc_feature: Yarrowia lipolytica putative sugar transporter YSP8
MSSISSSQQPNFKSILQYNARQEMIYWMAFVACLGTLQFGYHVAELNAPLDVIICHDAVTDCIDLHPRDVGYVTAAFSVGGLISALLAGMAASKYGPKKVSLFNTLSFIAGPLFMANATNTNTLAFGRFVSGLGAGAAIVVTPLFLNEIAPHNLRGMFGALSQISVNVGIVAAQVAGLIISQSWRYILIIGFFLGLINLASLAFIPESPKWLVSKNRATEATAILARLRDNRATAAREVEEWQKELQSVMFESDQLMAPASAASANHIEPVSLSAFLTRKPYRKPLLAILIIMTAQQLCGINSIIFYGVSILGQIIPKYSTLVNVFISVLGTVVTVGASRFIDVLGRKRLLLYSIFGMSVSAALVATGIINSWPIVSSLSAALFVVSFAIGLGPIPFLMVSEMVEPNCVGVGQSVGMTSNWIVTFAVGYFFPMVNARLGGATFYLFSVFGIAYLTLVVKFVPETKGKRNFTEVWTM

SEQ ID NO: 13 - protein, 565 aa, Yarrowia lipolytica
  misc_feature: Yarrowia lipolytica putative sugar transporter YSP10
MGFRGQRLHHYVATVAGMGFLLFGYDQGVMGGLLTLPSFVKQFPKMDTSDYLPPDVKSFNTTIQGTAIAIYEIGCMMGALFTMWGGDKVGRRYIIFYGSIIMTIGAVLQCASYSLGMFITGRVVSGVGNGFITATVPMLQSECAKPEKRGKLVMLEGALITAGIALSYWIDFGFYWVKANDADWRFPVAFQIVFCLFLFFTVLTIPESPRWLVKKGRFEEAAGVFAALEDVDIEDPYVVTQITYVKESIMLEQLAQLGIDGPAAREKIAAGEFSMGEELPFLSQMKLMFTFGKKKNFHRTMLAYWSQVMQQITGINLITYYAAYIYETSVGMTPTNSRILAACNGTEYFLASWIAFYTIERFGRRKLMIFGAAGQAATMAILTGCVYAASSPADGGLDNQSAGVAAAVFLFVFNTFFAIGWLGMSWLYPAEISSLEIRAPANGLSTSGNWVFNFMVVMITPVAFDTIKWKTYIIFAVINAAMVPVVYFFYPETAGRSLEEIDQIFADSNPKTPWDVVWIARRLPKSTAVDHNLVEPRILLEEKTALETVESVSPTPSDQEETRHV

SEQ ID NO: 14 - protein, 515 aa, Yarrowia lipolytica
  misc_feature: Yarrowia lipolyticaYHT1 - Strain E150
MGLANIINRGEKPEGSAFMAAFVAVFVAFGGILFGYDTGTISGVMAMPFVKKTFTDDGLEFTSEQTSLITSILSAGTFTGAISAPWASDTLGRRLGLILFCVVFSVGAILQTAATGRTLLIVGRVVAGLGVGGVSSIVPLYQSEVAPKWIRGAVVSIYQFAITIGLLLAAIVNNATKNKDNSASYRIPLGLQLLWAVILSGGLILLPETPRFWIKKGEYDKAADSLRRLRRLPVEHEAVQKELLEIQSSHDHEMQIGSATWAACFSPKGSQLKRMLTGIAIQALQQLTGINFIFYYGTEFFKKSNISNPFLIQMITNIVNVVMTIPGIMFVDRVGRRKLLLIGAIVMCSSEFIVAAVGTAIDNETSSKVLIAFTCTFIAGFAATWGPIAWVVIGEIFPLRIRAKGVALCAASNWLFNFAIAFATPYLVDEAPGSAGLKTKVFFIWGGCNFLCIAFTYFFIYETKGLTLEEVDQMYAEIKIASRSHQFVPTTRVAAYDEHASDDKKDGQHVYIESV

SEQ ID NO: 15 - protein, 515 aa, Yarrowia lipolytica
  misc_feature: Yarrowia lipolyticaYHT1 - Strain H222
MGLANIINRGEKPEGSAFMAAFVAVFVAFGGILFGYDTGTISGVMAMPFVKKTFTDDGLEFTSEQTSLITSILSAGTFTGAISAPWASDTLGRRLGLILFCVVFSVGAILQTAATGRTLLIVGRVVAGLGVGGVSSIVPLYQSEVAPKWIRGAVVSIYQFAITIGLLLAAIVNNATKNKDNSASYRIPLGLQLLWAVILSGGLILLPETPRFWIKKGEYDKAADSLRRLRRLPVEHEAVQKELLEIQSSHDHEMQIGSATWAACFSPKGSQLKRMLTGIAIQALQQLTGINFIFYYGTEFFKKSNISNPFLIQMITNIANVVMTIPGIMFVDRVGRRKLLLIGAIVMCSSEFIVAAVGTAIDNETSSKVLIAFTCTFIAGFAATWGPIAWVVIGEIFPLRIRAKGVALCAASNWLFNFAIAFATPYLVDEAPGSAGLKTKVFFIWGGCNFLCIAFTYFFIYETKGLTLEEVDQMYAEIKIASRSHQFVPTTRVAAYDEHASDDKKDGQHVYIESV

SEQ ID NO: 16 - protein, 515 aa, Yarrowia lipolytica; identical to SEQ ID NO: 14
  misc_feature: Yarrowia lipolyticaYHT1 - Strain W29

SEQ ID NO: 17 - protein, 494 aa, Yarrowia lipolytica
  misc_feature: Yarrowia lipolyticaYHT2 - Strain E150
MAIIVAVFVAFGGLLYGYDTGTIAGIMTMGYVKEHFTDFGKNDFTSGQSSLTTSILSVGTFTGAIVAPLAADTAGRRLGLLLYCLVFSVGAILQTVTTGRVLLIVGRVIAGLGVGGISSIVPLYQSEVSPKWIRGAVVSVYQFAITVGLLLAAIVNNATKDRPNTSSYRIPLGIQLIWALILSAGLVFLPETPRFWVKKNRPEKAAEALSRLRRLPTDSKPVKKELLELQKSFEMEMEVGNSSWKACFSPHGSQLKRLLTGVSIQALQQLTGINFIFYYGTNFFKTAGIKDPFVVSMITSAVNVAFTLPGILFVDKVGRRKLLLIGAVVMCVSELIVAAVGAALDSQVSSKVLIAFTCTFIAGFASTWGPIAWVVVAEIFPLRIRAKGVAISVAANWIFNFAIAFATPYLVDKKPGSAGLESKVFFIWGGCNFLAIAFVYLFVYETKGLSLEQVDEMYSEVKYAWQSDRFQTEIMSGKTEVSPDQSCDSGFDSD

SEQ ID NO: 18 - protein, 494 aa, Yarrowia lipolytica
  misc_feature: Yarrowia lipolyticYHT2 - Strain H222
MAIIVAVFVAFGGLLYGYDTGTIAGIMTMGYVKEHFTDFGKNDFTSGQSSLITSILSVGTFTGAIVAPLAADTAGRRLGLLLYCLVFSVGAILQTVTTGRVLLIVGRVIAGLGVGGISSIVPLYQSEVSPKWIRGAVVSVYQFAITVGLLLAAIVNNATKDRPNTSSYRISLGIQLIWALILSAGLVFLPETPRFWVKKNRPEKAAEALSRLRRLPTDSKPVKKELLELQKSFEMEMEVGNSSWKACFSPHGSQLKRLLTGVSIQALQQLTGINFIFYYGTNFFKTAGIKDPFVVSMITSAVNVAFTLPGILFVDKVGRRKLLLIGAVVMCVSELIVAAVGAALDSQVSSKVLIAFTCTFIAGFASTWGPIAWVVVAEIFPLRIRAKGVAISVAANWIFNFAIAFATPYLVDKKPGSAGLESKVFFIWGGCNFLAIAFVYLFVYETKGLSLEQVDEMYSEVKYAWQSDRFQTEIMSGKTEVSPDQSCDSGFDSD

SEQ ID NO: 19 - protein, 494 aa, Yarrowia lipolytica; identical to SEQ ID NO: 17
  misc_feature: Yarrowia lipolyticaYHT2 - Strain W29

SEQ ID NO: 20 - protein, 578 aa, Yarrowia lipolytica
  misc_feature: Yarrowia lipolytica putative sugar transporterYSP13
MKLQVPAFARSSSDVKTSFMGARGQKLHNLVAAIAGLGFLLFGYDQGVMGGLLTLDTFIQQFPKMDTSDYLPPKVKTFNTTIQGTAVGIYEIGCMIGALFTMWAGDKLGRRYMIFFGSIIMTIGAILQCASYSLGQFIAGRVISGIGNGFITATVPMLQSECAKPERRGKLVMLEGALITAGIALSYWIDFGFYWVRTNDADWRFPIAFQIVFSLVLTFTIMSLPESPRWLVKKQRFEEAAGVFAALEDVPLDDPYVINQITSVKESIMMEQLAQLGVDGVDARRKIQSGEFQMGQELSFIGQMKLMFTFGKKKNFHRTMLAYWNQVMQQVTGINLITYYAAYIYQTSVGMNATDSRILAACNGTEYFMASWVAFYTIERFGRRKLMLFGAVGQACTMAILTGCVYAASKPEDGGLDMQGAGIAAAVFLFVFNTFFAIGWLGMTWLYPAEISSLEIRAPANGLSTSGNWAFNFMVVMITPVAFNSIKWKTYIIFACINAFMVPMVYFFYPETAGRSLEEIDMIFAESNPRTPWDVVGIANRLPKNSVATYDDYEAGEQEEKAIVETAESVSHDSAEFQ

SEQ ID NO: 21 - protein, 659 aa, Yarrowia lipolytica
  misc_feature: Yarrowia lipolyticaYSP14
MVFGREKDDSEGIEHVPSPQDNPSDQTSDIIALNEKASNEHDDLPTIPKPEGDAPVNSELDPDNPLIRYSRAELLEIATQFAVDNDLADKAEAFRKGALVAQDPSGFENIDILDDDDRYWLNREITNKWDHPMKVYYLVVCCSLAAAVQGMDETVINGANIIFPAQFGIKEDSGVVSRKSWLLGLVNSAPYLCCACISCWMTDPINKVLGRKWTVFWTCFWAGATCFWSGFVNTWWHLFIARFFLGFGIGPKSATVPVYAAECAPPRIRGAMVMMWQMWTAFGIMMGYVMDLAFYYVKDRGTIVGLNWRLMLGSALIPALLVCIFIVKCPESPRWHLARGEIRKSFECMREIRHTDIQAARDTFYAHVLLIEENEMKKGKNRFVELFTVPRNRRAAWASFIVMFMQQFCGINVIAYYSSNIFMESGFGAIQALLASFGFGAINFVFALPAVYTIDTFGRRALLLATFPLMAIFLLFAGFCFYIGQNDPTHSHARVGLIALGIYLFSAVYSCGEGPVPFTYSAEAFPLYVRDLGMSFATAVCWLFNFVLAVTWPSLLAAFTPQGAFGWYAAWNVVGFFLVLCFLPETKNLTLEELDKVFSVPTRVHMKYQFNAFKINIQRTILRKDVPKPPPLYAHEAGIGGTSHWSSKPQPNANTAEFV

SEQ ID NO: 22 - protein, 539 aa, Yarrowia lipolytica
  misc_feature: Yarrowia lipolytica putative sugar transporter YSP15
MFWKNMKNEPKQVLNWTLWLSVLVFGLLGSARGLDEGNISGTSHQVSFENQFGLRDPNKTEKEIADALSNITAMVQLGSVGGSIIAMFAQDKIGRVRCLQQMLIVWIVGVVIQVTSHGQGQLLAGRFIAGLGIGQSVVIGPTYLAEVSPKNVRGLCTCVFSGSVYLGIMLEYFANYSTTLNVSDESRMQWVLPTSVQFIFAGLLFIGSFFIYESPRWLMKIGQEEKAMETLSKIRHLPIDDVYIQGEILDVREQVEREKQALSGTSIFSILKELVATQANRYRLFIGIMVQLLGQWSGANAVTIYSPQFFAMLGVTLRTDRMMYTAILGIIKFAASVVCAVFLIDTIGRRRSLYTGVCLQFISMLYLGIYLSIVPASSGAVEDRTASQRHAGGGAIAAIYLSGCGWALGWNSIQYLINAEIYTVRHRSLACGIIMVFHFVNQYGNSKALPYMRKSLTDHGTMFFFSGVILIGLAWSWFFLPEVSGRSLESIDEMFSLPWYLIGRRGAKLVPETSTTTQLQEDDDLAKEKKGATVHVETC

SEQ ID NO: 23 - protein, 592 aa, Yarrowia lipolytica
  misc_feature: Yarrowia lipolytica putative sugar transporter YSP16
MGRNWLAFSQASTEVKTEFLGLRGQKLHRAVAFIAGMGFLLFGYDQGVMGGLLTLPRFIHQFPKMDTSDYVPEETRKFNTTIQGVSVGIYEIGCMIGALFTMWAGDKLGRRRMIFWGSIIMTIGAILQCASYSLGQFITGRVVSGVGNGFITATVPMFQSECAKPERRGALVMMEGALITGGIALSYWIDFGFYWVNNDADWRFPIAFQIIFSMFLTFTVMSLPESPRWLVKKQRFDEAAGVFSALEDVPIDDPYVIDQIAEVKESIMMEQLAQLGVDGSEAREKIASGEFQMGAELPFLEQMKLLFTFGKKKNFHRTMLAYWNQVMQQITGINLITYYAAYIYETSVGMNATNSRILAACNGTEYFLASWVAFYTIERFGRRKLMLFGAIGQACTMAILTGTVYAASPPEDGGLDNSGAGIAAAVFLFVFNTFFAIGWLGMTWLYPAEITSLEIRAPANGLSTSGNWVFNFMVVMITPVAFDTIKWKTYIIFACINAAMVPVVYFFYPETAGRSLEEIDKIFAESNPRTPWDVVGIARRMPRESALTRRKQGVNQTFDNSNEKAVVEENTESAVGSTTGSASPSFENVSQA

SEQ ID NO: 24 - protein, 536 aa, Yarrowia lipolytica
  misc_feature: Yarrowia lipolytica putative sugar transporter YSP17
MFWKNMKNEPPQVLNWTLWLAVIVFGSLGSVRGLDEGLISGITSQASFESQFKLKDPLKSKSQQDDELSNITAMVQIGSVGGAMIAMLIQDKIGRIRSLQEMLILWTVGVIIEVTSYSQGQMLAGRLIAGLGIGQSVVIGPTYLAEVSPKNVRGLCTCIFSGSVYLGVMLEYFANYSTSMHMPATSRNQWVVPVSIQFIFAGLLFIGSFFVHESPRWLMKIGKDEEAIETLSKIRNLPADDLYVQGEIMDVREQIEREKQALSGTNIFSLMKELVSTKANRYRLFLGIMVQLLGQWSGANAVTVYSPKFFSMLGIPSKIDQMMYTAILGVIKLVSALSCALFLIDTIGRRRSLYTGICLQFVSMLYLGIYLAIVPAKTGVERTPSQRHAGAAAIAAIYLSGCGWALGWNSIQYLINAEIYTVRHRSLASGIIMTFHFANQYGNSKALPYMRAGITDHGTMFFFAGVLLLGFAWSWFFLPEVSGRSLESIDEMFSLPWYVIGRRGHKMIPETSATVHLRQEEDSESKKGGVVMVESC

SEQ ID NO: 25 - protein, 566 aa, Yarrowia lipolytica
  misc_feature: Yarrowia lipolytica putative sugar transporter YSP18
MSGQTYIEGWPGVEPVLFLLSLSTLSLSPPAMFWISMKNEPKQVLNRTLWFAIFVFGLLGTSRGLDEGNISGTVAQPSFISKFKLHDPSLSKDAQANLLSNITSMVQIGSVAGALIAMLIVDRIGRLWALREMLVIWIVGVIVQITSNNVGQLYAGRFVAGMGIGQSVVIGPTYLAEIAPPHVRGLATCVFSGSVYLGVMLSYFANYGTTLHISNTSRNQWVIPTTLQIIFSGIMLIGSIFVHESPRWLMKIGKEEEAVETLCKIRKLPADDLYVQGEILMVREQIEREKQAMTGASYWSLFKELLATPANRYRLFLGLMIQILGQWSGANAITIYAPSFFQMIGVSGQQQKMFATAILGVVKFAASIICAVFLIDTIGRRRSLYSGITLQFISILYVAIYLAVVPNNDPSRIKSAAERHAGVGAIAFIYLSGVGWALGWNSIQYLINAEIYTVRHRSLASGLIMTVHFANQYGNSKALPSMRLGMTDTGAMFFFSGIILVGLVWSWFFLPEVSGRSLESIDEMFSLPWYLIGRRGHQMVPETSAAVQIAADEDEKKGGVVHVENC

SEQ ID NO: 26 - protein, 565 aa, Yarrowia lipolytica; identical to SEQ ID NO: 13
  misc_feature: Yarrowia lipolytica hexose transporterYHT4 - Strain E150

SEQ ID NO: 27 - protein, 565 aa, Yarrowia lipolytica
  misc_feature: Yarrowia lipolytica hexose transporterYHT4 - Strain H222
MGFRGQRLHHYVATVAGMGFLLFGYDQGVMGGLLTLPSFVKQFPKMDTSDYLPPDVKSFNTTIQGTAIAIYEIGCMMGALFTMWGGDKVGRRYIIFYGSIIMTIGAVLQCASYSLGMFITGRVVSGVGNGFITATVPMLQSECAKPEKRGKLVMLEGALITAGIALSYWIDFGFYWVKANDADWRFPVAFQIVFCLFLFFTVLTIPESPRWLVKKGRFEEAAGVFAALEDVDIEDPYVVTQITYVKESIMLEQLAQLGIDGPAAREKIAAGEFSMGEELPFLSQMKLMFTFGKKKNFHRTMLAYWSQVMQQITGINLITYYAAYIYETSVGMTPTNSRILAACNGTEYFLASWIAFYTIERFGRRKLMIFGAAGQAATMAILTGCVYAASSPADGGLDNQSAGVAAAVFLFVFNTFFAIGWLGMSWLYPAEISSLEIRAPANGLSTSGNWVFNFMVVMITPVAFDTIKWKTYIIFAVINAAMVPVVYFFYPETAGRSLEEIDQIFADSNPKTPWDVVWIARRLPKSTAVDHNLVEPRILHEEKTALETVESVSPTPSDQEETRHV

SEQ ID NO: 28 - protein, 565 aa, Yarrowia lipolytica; identical to SEQ ID NO: 13
  misc_feature: Yarrowia lipolytica hexose transporterYHT4 - Strain W29

SEQ ID NO: 29 - protein, 532 aa, Yarrowia lipolytica
  misc_feature: Yarrowia lipolytica putative sugar transporter YSP20
MFSLTGKPLLYFTSVFVSLGVFLFGYDQGVMSGIITGFYFKEYFHEPTRAEIGTMVSILEVGAFVSSLMVGRIGDIIGRRKTIMYGAFIFIIGGAFQTFAVSMSEMILGRVVAGFGVGMLSTIVPVYQSEISPPHNRGKLACIEFTGNIVGYASSVWVDYFCSFINSNMSWRIPLFLQCAMGALLFGGSFLIAETPRWLLDNDHDEEGLVVLANLHGGGDIDSPLAKQEYREIKQSVLIHRLEGERSYTDMWKKYKKRVLIAMSSQMFAQLNGINVISYYAPLVFEEAGWVGRSAILMTGINGIVYVCSTIPPWYLVDKWGRRPILLSGAVIMAISLASVAFWMRLDFAHTPALVVISVVIFNAAFGYSWGPIPWLYPPEIMPLTIRAKGASLSTATNWAFNWLVGYMTPILQETIKWRLYLMHAAFCSLSFVLVYFTYPETSGINLEDMDSLFGDKSVVNTPDSRSLLGDRDTPEPDVPHSYTDAATDRLPAGMQGYGSAPSSRGGSVVGSPRRGNSVVGSPKRDFPQPPV

SEQ ID NO: 30 - protein, 659 aa, Yarrowia lipolytica
  misc_feature: Yarrowia lipolytica putative sugar transporter YSP21
MSSYPSEKGGTEGIDRVPSETNDASDNTSDNNGLHEKPSNEHAEGLPPGNALNADLDPENPLSRYTRDELLEIASGFAKENGMGDKEDIFRKGALVAQDPANFDNIDILDDNDRYWLRREITHKWDHPMKVYYIVICCSLAAAVQGMDETVINGANIIFPAQFGIKEEAGVVSQKTWLLGLVNSAPYLCCAVVSCWLTDPINRLLGRKWTIFWTCFWSGATCFWSGFVNNWWHLFIARFFLGFGIGPKSATVPVYAAECAPPTIRGAMVMMWQMWTAFGIMMGYVMDLAFYYVPDHGIEGLNWRLMLGSALIPALLVCVQVIWCPESPRWHLARGEISKAYECMRVIRNSEVQAARDLFYAHVLLLEEEELKRGKNRFFELFTVPRNRRASWASFIVMFMQQFCGINVIAYYSSNIFMESGFGEIQALLASFGFGAINFVFALPAVYTIDTFGRRALLLVTFPLMAIFLLFAGFCFWIDQDDPTNSPARVGCIALGIYLFSAVYSCGEGPVPFTYSAEAFPLYIRDLGMSFATATCWLFNFVLAVTWPSLLAAFTPQGAFGWYAAWNVVGFFLVLCFLPETKNLTLEELDKVFGVPTRVHMKYQFNAFKVNIQRHLFRQDIPKPPPLYAHEVGVGGTSHYQHKPHHTANLTPLHTAELA

SEQ ID NO: 31 - protein, 533 aa, Yarrowia lipolytica
  misc_feature: Yarrowia lipolytica YHT3 - Strain E150
MSTSAMTDDYPMAEVSITEETKMPEPAKKSQLLVILLCLFAAMGGFVFGYDTGTISGFINMDPFLESFGEADESGVFFLSRIRAGLIVGLFSIGAFLGTLLGGFLADKVGRKKGIVYVAMVYIVGMLIQITAFTAWYQIAIGRVIGGVGIGALSVLVPMFQSETAPQNLRGALVSSFQLFITLGIFIGFSVCYATKSRLDTGAYRIPMGLCFAWAFILLIGMCFMPESPRFLVSIGRIEEARKAMAMTNQVPLHDAVIDEELKAIENSVIREKSAGKATWKELFTGEPRMGYRLTLGILVQVLQQLCGANYFFYYGTSIFKAIGMSDSFATSMIFGGINLLSTFGGLYIVDRFGRRKCLLGGAMVMFVCFLVYSTVGFAALYPNGDTTLPARKAVGDVMILFTCIFIAAFASTWAPIAFVVVSETFPLRMRSKGMAVATGGNWMINFLVSFLTPFITSSIGFKYGYVFTACIGFAIIFVFFFIPETKGLSLEDVDELYASGVSARNSPNWVPSSKKQEVNEPKNSMTDFSESS

SEQ ID NO: 32 - protein, 533 aa, Yarrowia lipolytica; identical to SEQ ID NO: 31
  misc_feature: Yarrowia lipolytica YHT3 - strain H222

SEQ ID NO: 33 - protein, 533 aa, Yarrowia lipolytica
  misc_feature: Yarrowia lipolytica  YHT3 - strain W29
MSTSAMTDDYPMAEVSITEETKMPEPAKKSQLLVILLCLFAAMGGFVFGYDTGTISGFINMDPFLESFGEADESGVFFLSRIRAGLIVGLFSIGAFLGTLLGGFLADKVGRKKGIVYVAMVYIVGMLIQITAFTAWYQIAIGRVIGGVGIGALSVLVPMFQSETAPQNLRGALVSSFQLFITLGIFIGFSVCYATKSRLDTGAYRIPMGLCFAWAFILLIGMCFMPESPRFLVSIGRIEEARKAMAMTNQVPLHDAVIDEELKAIENSVIREKSAGKATWKELFTGEPRMGYRLTLGILVQVLQQLCGANYFFYYGTSIFKAIGMSDSFATSMIFGGINLLSTFGGLYIVDRFCRRKCLLGGAMVMFVCFLVYSTVGFAALYPNGDTTLPARKAVGDVMILFTCIFIAAFASTWAPIAFVVVSETFPLRMRSKGMAVATGGNWMINFLVSFLTPFITSSIGFKYGYVFTACIGFAIIFVFFFIPETKGLSLEDVDELYASGVSARNSPNWVPSSKKQEVNEPKNSMTDFSESS

SEQ ID NO: 34 - protein, 540 aa, Yarrowia lipolytica
  misc_feature: Yarrowia lipolytica putative sugar transporterYSP23
MSLDKNRQITTSESSSGSSADEGTHIMRGLTSTSTQDETTGSISEGNLEAEKISPFVFVLVALASISGFLFGYDTGYVSGALVVIKEDLGRALSNGDKELITASTSLGALLGGVIAGAMCDFFGRKWVITFANILFLVGAAIQCGAHAVWTMIGGRFVMGWGVGIASLCAPLYISELAPTRIRGRLVVLNVLAITGGQLVAYGIGAGMAHVHQGWRILVGLSMVPAFVQMVIFVFMPETPRYLVRKNKIAEAKKVLAKTYATDDDNLLDRKLHELMLHNAYKESGLSTMARARNTMKELYCVPSNLRALIIACGLQGIQQFCGFNSLMYFSATIFEVVGFDNATAVSIIVAGTNFVFTIVAFMVIDRIGRRRILLGTIWGMSLGLVVNAIAFHFLDKQKEKNPNHELDKEHISGWAYVVLVAQLVYVAFYATGIGNVPWQQSELFPISVRGVGTGMATATNWAGSLIVSSTFLTMLENITPTGTFSFYAGLCALGEVFVFFLYPETSGMDLEQIQQLLTGGFNIKESMRLSDEAKRGYKK

SEQ ID NO: 35 - protein, 599 aa, Yarrowia lipolytica
  misc_feature: Yarrowia lipolytica putative sugar transporter YSP24
MILFWLHRGVFALSEYRIYIRGQVPRSRSLFLTARLTMLFSKASGTKYFGLKGKTLQRAIGGIAGLGFFLFGYDQGVMGGLLNLKTFREQFETINTVDDTSLHTATIQGTAIAVYELGCMVGALSTIYLGDKLGRRKVIYFGTWIIIIGAIIQTASYHLGQLIVGRVVAGIGNGLITATVPMWQSECARPEDRGLMVMIEGCLISGGIALSYWLDFAFFFVKVGSMDWRFPVAFQILIALIIVAFVLEFPESPRWLVKVGREEDAREVWAALENCGPNDDYINYEIHEVKKNLAAEEQMTSWQTFKLIFTYGERKHFHRACLAFWNQAMQQLTGINLITYYAAKIYQDSLHMDDVVSRALAAANGTEYFMASFIPFWSIERFGRRKLMLFGAVGQACTMGILTGTAWAADPNNKDNRAAGIAACVFLFVFNSFFAIGWLGMTWLYPAEITSLEVRAPVSGISTASNWLFNFTVVMICPVGFNSIQSYTYTIFAAINACMVPVIYFLYPETAGRSLEEIDEIFKESNPKTPWDVVGIAARMPKRDHYNYDIESRGNSMGEQYPEGEPKPEHEEVPEGYTSETAGENSNSSVEYPDTTTHQ

SEQ ID NO: 36 - protein, 515 aa, Yarrowia lipolytica
  misc_feature: Yarrowia lipolyticaYHT1-I161V - Strain H222
MGLANIINRGEKPEGSAFMAAFVAVFVAFGGILFGYDTGTISGVMAMPFVKKTFTDDGLEFTSEQTSLITSILSAGTFTGAISAPWASDTLGRRLGLILFCVVFSVGAILQTAATGRTLLIVGRVVAGLGVGGVSSIVPLYQSEVAPKWIRGAVVSIYQFAVTIGLLLAAIVNNATKNKDNSASYRIPLGLQLLWAVILSGGLILLPETPRFWIKKGEYDKAADSLRRLRRLPVEHEAVQKELLEIQSSHDHEMQIGSATWAACFSPKGSQLKRMLTGIAIQALQQLTGINFIFYYGTEFFKKSNISNPFLIQMITNIANVVMTIPGIMFVDRVGRRKLLLIGAIVMCSSEFIVAAVGTAIDNETSSKVLIAFTCTFIAGFAATWGPIAWVVIGEIFPLRIRAKGVALCAASNWLFNFAIAFATPYLVDEAPGSAGLKTKVFFIWGGCNFLCIAFTYFFIYETKGLTLEEVDQMYAEIKIASRSHQFVPTTRVAAYDEHASDDKKDGQHVYIESV

SEQ ID NO: 37 - protein, 533 aa, Yarrowia lipolytica
  misc_feature: Yarrowia lipolyticaYHT3-I181V - Strain H222
MSTSAMTDDYPMAEVSITEETKMPEPAKKSQLLVILLCLFAAMGGFVFGYDTGTISGFINMDPFLESFGEADESGVFFLSRIRAGLIVGLFSIGAFLGTLLGGFLADKVGRKKGIVYVAMVYIVGMLIQITAFTAWYQIAIGRVIGGVGIGALSVLVPMFQSETAPQNLRGALVSSFQLFVTLGIFIGFSVCYATKSRLDTGAYRIPMGLCFAWAFILLIGMCFMPESPRFLVSIGRIEEARKAMAMTNQVPLHDAVIDEELKAIENSVIREKSAGKATWKELFTGEPRMGYRLTLGILVQVLQQLCGANYFFYYGTSIFKAIGMSDSFATSMIFGGINLLSTFGGLYIVDRFGRRKCLLGGAMVMFVCFLVYSTVGFAALYPNGDTTLPARKAVGDVMILFTCIFIAAFASTWAPIAFVVVSETFPLRMRSKGMAVATGGNWMINFLVSFLTPFITSSIGFKYGYVFTACIGFAIIFVFFFIPETKGLSLEDVDELYASGVSARNSPNWVPSSKKQEVNEPKNSMTDFSESS

SEQ ID NO: 38 - DNA, 37 bp, artificial
  ylHXK1-forward primer
gagaagatctatggttcatcttggtccccgaaaaccc

SEQ ID NO: 39 - DNA, 38 bp, artificial
  ylHXK1-reverse primer
gcgccctaggctaaatatcgtacttgacaccgggcttg

SEQ ID NO: 40 - DNA, 36 bp, artificial
  scHXK2-forward primer
gcgcggatccatggttcatttaggtccaaaaaaacc

SEQ ID NO: 41 - DNA, 32 bp, artificial
  scHXK2-reverse primer
gcgccctaggttaagcaccgatgataccaacg

SEQ ID NO: 42 - DNA, 20 bp, artificial
  ylHXK1-qPCR-forward primer
tctcccagcttgaaaccatc

SEQ ID NO: 43 - DNA, 20 bp, artificial
  ylHXK1-qPCR-reverse primer
cttgacaactcgcaggttgg

SEQ ID NO: 44 - DNA, 35 bp, artificial
  A01958-forward primer
gcgcactagtatgttctggaagaacatgaaaaatg

SEQ ID NO: 45 - DNA, 36 bp, artificial
  A01958-reverse primer
gcgcaagcttttaacaattctccacatgaataacac

SEQ ID NO: 46 - DNA, 33 bp, artificial
  A08998-forward primer
gcgcactagtatgaagctgtttaaacgagaagc

SEQ ID NO: 47 - DNA, 33 bp, artificial
  A08998-reverse primer
gcgcaagcttctatccacgaatagtggcacctc

SEQ ID NO: 48 - DNA, 35 bp, artificial
  A14212-forward primer
gcgcactagtatgtcaatcaagtcgctctcaaagg

SEQ ID NO: 49 - DNA, 35 bp, artificial
  A14212-reverse primer
gcgcaagcttctagacaccatctttagcaaccttc

SEQ ID NO: 50 - DNA, 28 bp, artificial
  B00396-forward primer
gagaactagtatgtcgcaccggccctgg

SEQ ID NO: 51 - DNA, 38 bp, artificial
  B00396-reverse primer
gcgcaagctttcacctatcagcattttcacccatttcc

SEQ ID NO: 52 - DNA, 37 bp, artificial
  B01342-forward primer
gcgcactagtatgtacaaggtccataacccctacctc

SEQ ID NO: 53 - DNA, 35 bp, artificial
  B01342-reverse primer
gcgcaagcttttagacatgctcagttccaggatac

SEQ ID NO: 54 - DNA, 35 bp, artificial
  B06391-forward primer
gcgcactagtatgattggaaacgctcaaattaacc

SEQ ID NO: 55 - DNA, 35 bp, artificial
  B06391-reverse primer
gcgcaagcttttacaattgagagggaggggcgtcg

SEQ ID NO: 56 - DNA, 33 bp, artificial
  B17138-forward primer
gcgcactagtatgaaagacttcctcgccttcac

SEQ ID NO: 57 - DNA, 31 bp, artificial
  B17138-reverse primer
gcgcaagcttctacgctgtctcgattcgaac

SEQ ID NO: 58 - DNA, 37 bp, artificial
  B21230-forward primer
gcgcactagtatgtcgtctatatcttcgtcccagcag

SEQ ID NO: 59 - DNA, 40 bp, artificial
  B21230-reverse primer
gcgcaagcttctacatggtccaaacctcggtaaaatttcg

SEQ ID NO: 60 - DNA, 31 bp, artificial
  C04686-forward primer
gcgcactagtatgtcgctggctatcaccaac

SEQ ID NO: 61 - DNA, 36 bp, artificial
  C04686-reverse primer
gcgcaagcttttaagctggctgagtagtgttattgg

SEQ ID NO: 62 - DNA, 32 bp, artificial
  C04730-forward primer
gcgcactagtatgggcttcagaggccaaagac

SEQ ID NO: 63 - DNA, 42 bp, artificial
  C04730-reverse primer
gcgcaagcttttaaacatgtctggtttcctcttgatcagaag

SEQ ID NO: 64 - DNA, 31 bp, artificial
  C06424-forward primer
gcgcactagtatgggactcgctaacatcatc

SEQ ID NO: 65 - DNA, 40 bp, artificial
  C06424-reverse primer
gcgcaagcttctagacagactcaatgtagactgtctgtcc

SEQ ID NO: 66 - DNA, 35 bp, artificial
  C08943-forward primer
gcgcggatccatggccattattgtggctgtatttg

SEQ ID NO: 67 - DNA, 35 bp, artificial
  C08943-reverse primer
gcgcatcgatctaatccgaatcaaatccagaatcg

SEQ ID NO: 68 - DNA, 35 bp, artificial
  C16522-forward primer
gcgcactagtatgaagctacaagtacccgcgtttg

SEQ ID NO: 69 - DNA, 31 bp, artificial
  C16522-reverse primer
gagagtcgactcactgaaactcggccgaatc

SEQ ID NO: 70 - DNA, 34 bp, artificial
  D00132-forward primer
gcgcactagtatggtttttggacgagaaaaagac

SEQ ID NO: 71 - DNA, 29 bp, artificial
  D00132-reverse primer
gcgcaagcttttaaacgaactcggcagtg

SEQ ID NO: 72 - DNA, 37 bp, artificial
  D00363-forward primer
gcgcactagtatgttctggaaaaacatgaagaatgag

SEQ ID NO: 73 - DNA, 31 bp, artificial
  D00363-reverse primer
gagagtcgacctaacaggtctccacgtgaac

SEQ ID NO: 74 - DNA, 29 bp, artificial
  D01111-forward primer
gcgcactagtatgggacgaaactggctag

SEQ ID NO: 75 - DNA, 36 bp, artificial
  D01111-reverse primer
gcgccccgggttaagcttgagaaacgttctcaaaag

SEQ ID NO: 76 - DNA, 35 bp, artificial
  D18876-forward primer
gcgcactagtatgttctggaaaaatatgaagaatg

SEQ ID NO: 77 - DNA, 29 bp, artificial
  D18876-reverse primer
gcgcaagcttctaacacgactccaccatc

SEQ ID NO: 78 - DNA, 32 bp, artificial
  E20427-forward primer
gcgcactagtatgtccgggcagacatatatag

SEQ ID NO: 79 - DNA, 29 bp, artificial
  E20427-reverse primer
gagagtcgacctagcagttctccacatgg

SEQ ID NO: 80 - DNA, 30 bp, artificial
  E23287-forward primer
gcgcactagtatggcgaggctttgtctttc

SEQ ID NO: 81 - DNA, 34 bp, artificial
  E23287-reverse primer
gcgcaagcttttaaacagtctcggtgtactgagg

SEQ ID NO: 82 - DNA, 33 bp, artificial
  F06776-forward primer
gcgcactagtatgttttcgttaacgggcaaacc

SEQ ID NO: 83 - DNA, 34 bp, artificial
  F06776-reverse primer
gcgcaagcttttataccggaggttgagggaagtc

SEQ ID NO: 84 - DNA, 34 bp, artificial
  F18084-forward primer
gcgcactagtatgtcttcctatccatccgagaag

SEQ ID NO: 85 - DNA, 31 bp, artificial
  F18084-reverse primer
gagtaagcttttaagcaagctccgccgtgtg

SEQ ID NO: 86 - DNA, 30 bp, artificial
  F19184-forward primer
gcgcggatccatgtccactagtgctatgac

SEQ ID NO: 87 - DNA, 31 bp, artificial
  F19184-reverse primer
gcgcaagcttctaagaggactcggagaagtc

SEQ ID NO: 88 - DNA, 29 bp, artificial
  F23903-forward primer
gcgcggatccatgtcgctggacaaaaacc

SEQ ID NO: 89 - DNA, 33 bp, artificial
  F23903-reverse primer
gcgcaagcttctacttcttgtagcctctcttgg

SEQ ID NO: 90 - DNA, 43 bp, artificial
  F25553-forward primer
gcgcactagtatgatacttttttggttacacagaggcgtcttc

SEQ ID NO: 91 - DNA, 38 bp, artificial
  F25553-reverse primer
gcgcaagcttttattgatgagtggtggtgtcggggtac

SEQ ID NO: 92 - DNA, 28 bp, artificial
  C06424-I162V-forward primer
cagtttgccgtcaccattggtcttctgc

SEQ ID NO: 93 - DNA, 28 bp, artificial
  C06424-I162V-reverse primer
gcagaagaccaatggtgacggcaaactg

SEQ ID NO: 94 - DNA, 28 bp, artificial
  F19184-I181V-forward primer
ccagctgtttgttactctcggcatcttc

SEQ ID NO: 95 - DNA, 28 bp, artificial
  F19184-I181V-reverse primer
gaagatgccgagagtaacaaacagctgg

(19) United States
(12) Patent Application Publication
(10) Pub. No.: US 2017/0145449 A1
(43) Pub. Date: May 25, 2017

(54) Improved lipid accumulation in Yarrowia lipolytica strains by overexpression of hexokinase and new strains thereof

(71) Applicant: INSTITUT NATIONAL DE LA RECHERCHE AGRONOMIQUE, Paris (FR)

(72) Inventors: Jean-Marc NICAUD, Trappes (FR); Zbigniew LAZAR, Wroclaw (PL); Thierry DULERMO, Saint Germain en Laye (FR); Anne-Marie CRUTZ-LE COQ, Velizy (FR)

(21) Appl. No.: 15/316,392

(22) PCT Filed: Jun. 11, 2015
(86) PCT No.: PCT/EP2015/063102

§ 371 Date: Dec. 5, 2016

(30) Foreign Application Priority Data
Jun. 11, 2014 (EP) 14305881.6

(51) Int. Cl. C12P 7/64; C07K 14/395; C12N 15/81; C12N 9/04; C12N 9/10; C12N 9/12; C12N 9/26

(52) U.S. Cl. CPC C12P 7/6409; C12N 9/1205; C12Y 207/01001; C07K 14/395; C12N 9/2431; C12Y 302/01026; C12N 9/0006; C12Y 101/01008; C12N 9/1029; C12Y 203/0102; C12P 7/6427; C12N 15/815

(57) ABSTRACT

The present invention relates to oleaginous yeast strains overexpressing a hexokinase gene, wherein said strains are capable of accumulating lipids. Methods for obtaining said strains as well as methods for producing lipids are also disclosed.

Several technologies such as large-scale fermentation are used for the industrial production of oil from microorganisms by using fatty substances or glycerol as a substrate. Within the framework of these projects, the microorganisms are used as a cell factory by redirecting the metabolism thereof to the production of compounds of industrial or dietary interest, such as waxy esters, isoprenoids, polyhydroxyalkanoates and hydroxylated fatty acids. The majority of these target the production of reserve lipids with a specific structure and/or composition. These include essential polyunsaturated fatty acid-enriched oils, which can potentially be used as a food supplement, lipids having compositional similarities with cocoa butter and non-specific oils intended for use in synthesizing biofuels.

Consequently, a growing interest is being observed in improving the composition and oil content of microorganisms, particularly yeasts.

One of the most studied and used oleaginous microorganisms is the yeast Yarrowia lipolytica, which can accumulate cell lipids of up to 40% of its dry weight. In contrast, the standard reference yeast Saccharomyces cerevisiae, which is not oleaginous, can only accumulate lipids in amounts of up to 15% over its own biomass (Dyer et al., 2002). The fully sequenced Y. lipolytica genome (Dujon et al., 2004) has served as a valuable tool. It has enabled the improvement of some aspects of lipid metabolism through the manipulation of several genes involved in the bioconversion, synthesis, and mobilization of lipids (Beopoulos et al., 2008; Dulermo and Nicaud, 2011; Beopoulos et al., 2012; Tai and Stephanopoulos, 2013; Blazeck et al., 2014). However, despite the increasing amount of information available on the biosynthesis of triacylglycerols and steryl esters, the rate-limiting steps in the lipid production process have yet to be identified.

Yeasts, in particular Y. lipolytica, begin to accumulate lipids when nitrogen in the medium is limiting and carbon resources are in excess. Specifically, yeasts under nutriment limitation undergo three phases of growth: (i) cell proliferation or the exponential growth phase, (ii) a lipid accumulation phase where growth slows down due to nutriment (i.e. nitrogen) limitation and lipid synthesis is maximal and (iii) a late accumulation phase where lipids continue to accumulate, but β-oxidation, the catabolic (break down) pathway is active in an effort to remobilize the carbon stored. Finally, cells become unable to produce essential metabolites and most of metabolic activity ceases. The process depends on temperature and pH and is also competitive with the production of citric acid, an immediate precursor of lipid accumulation. The C/N ratio of the medium affects various metabolic parameters, such as growth, organic acid production, and lipid biosynthesis (Beopoulos et al., 2009).

The efficiency of carbon source utilization is therefore an important factor in biomass production and lipid accumulation.

Glucose and fructose, widespread in nature and easy to produce industrially, are relatively cheap raw materials for the production of intracellular lipids. Both monosaccharides are also components of the disaccharide sucrose (table sugar), a readily available compound that has already been successfully used in citric acid production by genetically modified strains of Y. lipolytica (Lazar et al., 2011, 2013; Moeller et al., 2012).

It is thus highly desirable that both glucose and fructose be utilized as efficiently as possible by the yeast in order to maximize the ratio of lipids produced by hexose consumed.

However, this process has revealed some issues related to the use of fructose: it appears that glucose is preferentially consumed over fructose and, therefore, fructose is only used after any available glucose has been completely consumed (Lazar et al., 2011; 2013). Fructose is thus utilized late in the production process and may not be completely consumed before cell growth is inhibited, partially due to citric acid production (Lazar et al., 2011). A similar situation occurs during ethanol fermentation of grape must by S. cerevisiae and can lead to fermentation defects (Liccioli et al., 2011). In both species, strains with different fructose utilization capacities have been characterized (Guillaume et al., 2007; Lazar et al., 2011; Liccioli et al., 2011).

There is thus still a need for a yeast strain capable of accumulating lipids which can utilize both glucose and fructose efficiently.

### DESCRIPTION

The present inventors have now identified the formation of fructose-6-phosphate as a key limiting step for the accumulation of lipids in oleaginous organisms.

Phosphorylation of hexoses, e.g., glucose and fructose, is one of the key steps in sugar metabolism. This process is carried out by specific kinases in the hexokinase gene family, namely, glucokinase, which is specialized for glucose phosphorylation, and hexokinase, which is involved in the phosphorylation of other hexoses, including fructose.

The present inventors have now shown that the formation of the fructose-6-phosphate is crucial for lipid production in yeasts.

Indeed, they have shown that hexokinase plays an important role in lipid accumulation in yeasts, particularly in oleaginous yeasts such as Y. lipolytica. Overexpression of a hexokinase gene leads to increased hexokinase activity and thereby improved fructose uptake. Importantly, hexokinase overexpression triggers enhanced biomass production and lipid accumulation.

Thus in first embodiment, the present invention relates to a yeast strain overexpressing a hexokinase gene, said strain being capable of accumulating lipids.

Within the meaning of the present invention, the term “yeast” is understood to mean yeast strains in general, i.e., this term includes, among others, S. cerevisiae, Saccharomyces sp., Hansenula polymorpha, Schizzosaccharomyces pombe, Y. lipolytica, Pichia pastoris, Pichia finlandica, Pichia trehalophila, Pichia koclamae, Pichia membranaefaciens, Pichia minuta (Ogataea minuta, Pichia linderneri), Pichia opuntiae, Pichia thermotolerans, Pichia salictaria, Pichia guercuum, Pichia pijperi, Pichia stiptis, Pichia methanolica, Pichia sp., Metschnikowia puicherrima, Kluyveromyces sp., Kluyveromyces lactis, Candida albicans.

According to the invention, the yeast is preferably an oleaginous yeast (Ratledge, in: Ratlege C, Wilkinson S G editors, Microbial lipids, Vol. 2. London: Academic press 1988). The term “oleaginous” refers to those organisms that tend to store their energy source in the form of oil (Weete, In: Fungal Lipid Biochemistry, 2^{nd} Ed., Plenum, 1980). More specifically, an “oleaginous yeast” according to the invention is a yeast that can make oil. Generally, the cellular oil content of oleaginous microorganisms follows a sigmoid curve, wherein the concentration of lipid increases until it reaches a maximum at the late logarithmic or early stationary growth phase and then gradually decreases during the late stationary and death phases (Yongmanitchai and Ward, 1991). It is common for oleaginous microorganisms to accumulate in excess of about 25% of their dry cell weight as oil. The most widely known oleaginous yeasts include the genera Candida, Cryptoccocus, Rhodotorula, Rhizopus, Trichosporon, Lypomyces and Yarrowia. The particularly preferred yeasts, within the meaning of the invention, include Y. lipolytica, Rhodotura glutinis and Rhodosporidium torulides. A preferred yeast within the meaning of the present invention is Y. lipolytica. Most preferably, said Y. lipolytica strain has an A101, a H222 or a W29 background.

The present invention therefore preferentially relates to an oleaginous yeast strain overexpressing a hexokinase gene, said mutant strain being capable of accumulating lipids.

The term “overexpression” as used herein, refers to the increased expression of a polynucleotide encoding a protein. The term “expression”, as used herein, refers to the transcription and stable accumulation of sense (mRNA) or antisense RNA. Expression also includes translation of mRNA into a polypeptide. The term “increased” as used in certain embodiments means having a greater quantity, for example a quantity only slightly greater than the original quantity, or for example a quantity in large excess compared to the original quantity, and including all quantities in between. Alternatively, “increased” may refer to a quantity or activity that is at least 1%, 2%, 3%, 4%, 5%, 6%, 7%, 8%, 9%, 10%, 11%, 12%, 13%, 14%, 15%, 16%, 17%, 18%, 19% or 20% more than the quantity or activity for which the increased quantity or activity is being compared. The terms “increased”, “greater than”, and “improved” are used interchangeably herein.

A “hexokinase” according to the invention is an enzyme which phosphorylates a hexose to yield a hexose phosphate (EC number: 2.7.1.1). Within the hexokinase family, two different types of enzymes can be distinguished on the basis of their preferred substrates. Glucokinase is specialized for glucose phosphorylation, while hexokinase is involved in the phosphorylation of other hexoses, including fructose. Preferably, a hexokinase according to the invention is not a glucokinase. According to this specific embodiment, the oleaginous yeast strain of the invention overexpresses a non-glucokinase hexokinase gene, and is capable of accumulating lipids. In a further preferred embodiment, the oleaginous yeast strain of the invention is a Y. lipolytica strain overexpressing a non-glucokinase hexokinase gene, and capable of accumulating lipids.

In S. cerevisiae, glucose phosphorylation at position C6 is catalyzed by two hexokinases (i.e., Hxk1 and Hxk2) and a glucokinase (i.e., GlkI). Likewise, a hexokinase and a glucokinase have been experimentally identified in Y. lipolytica (Petit and Gancedo, 1999) and are encoded by YALIOB22308g (ylHXK1) and YALI0E15488g (ylGLK1), respectively.

The ylHXK1 gene encodes a hexokinase catalyzing the phosphorylation of hexoses with the exception of glucose, notably fructose. The sequence of the said ylHXK1 gene is represented by SEQ ID NO: 1 and is accessible under the accession number YALI0B22308g at the address: http://gryc.inra.fr/ (formerly www.genolevures.org). The sequence of the hexokinase encoded by ylHXK1 gene is represented by SEQ ID NO: 2. Y. lipolytica hexokinase has been shown to be the functional equivalent of S. cerevisiae hexokinase II (scHXK2p, YGL253W), which is involved in glucose catabolite repression (Petit and Gancedo, 1999); in addition, ylHXK1 is suspected to be involved in glucose repression of the LIP2 gene, which encodes extracellular lipase in Y. lipolytica (Fickers et al., 2005a).

Overexpression of the ylHXK1 gene is particularly advantageous for obtaining high amounts of lipids in an oleaginous yeast, such as Y. lipolytica, grown on fructose. Indeed, overexpression of an endogenous HXK2 gene has no effect on S. cerevisiae growth on fructose, while overexpressing a hexokinase gene, such as ylHXK1, in an oleaginous yeast results in a clear stimulation of fructose assimilation and, ultimately, in lipid accumulation.

In a preferred embodiment, the hexokinase gene is ylHXK1 and the invention relates to an oleaginous yeast strain, more particularly a Y. lipolytica strain, overexpressing ylHXK1, said strain being capable of overexpressing lipids. Most preferably, said Y. lipolytica strain has an A101, a H222 or a W29 background.

The selection of the carbon source which is to be used is of great importance for optimizing lipid production by the oleaginous yeast of the invention. In this regard, the strain of the invention is highly advantageous since it is capable of generating high amounts of biomass when grown on fructose as a carbon source. In particular, the inventors showed that ylHXK1 is crucial for fructose assimilation in Y. lipolytica. Overexpression of Y. lipolytica hexokinase results in increased biomass production and improved lipids yield.

The term “biomass” refers to material produced by growth and/or propagation of cells. Biomass may contain cells and/or intracellular contents as well as extracellular material. Extracellular material includes, but is not limited to, compounds secreted by a cell.

As explained above, and as shown in more details in the examples, a large proportion of the biomass produced by the oleaginous yeast strain of the present invention is constituted by lipids, i.e., the strain of the present invention is capable of producing significant levels of lipids.

By “lipids”, it is herein referred to any fat-soluble (i.e., lipophilic), naturally-occurring molecule. Lipids are a diverse group of compounds that have many key biological functions, such as structural components of cell membranes, energy storage sources and intermediates in signaling pathways. Lipids may be broadly defined as hydrophobic or amphiphilic small molecules that originate entirely or in part from either ketoacyl or isoprene groups. For a general overview of all lipid classes, refer to the Lipid Metabolites and Pathways Strategy (LIPID MAPS) classification system (National Institute of General Medical Sciences, Bethesda, Md.). The term “oil” refers to a lipid substance that is liquid at 25° C. and usually polyunsaturated. In oleaginous organisms, oil constitutes a major part of the total lipid and is composed primarily of triacylglycerols. Indeed, oleaginous yeasts store their lipids mostly in the form of TAG (80-90% of the neutral lipid fraction) and the rest in the form of steryl esters (SE). As used herein, the term “triacylglycerols” (TAGs) is synonymous with the term “triacylglycerides” and refers to neutral lipids composed of three fatty acyl residues esterified to a glycerol molecule. TAGs can contain long chain polyunsaturated fatty acids (PUFAs) and saturated fatty acids, as well as shorter chain saturated and unsaturated fatty acids.

In yeasts, triglyceride synthesis follows the Kennedy pathway. The free fatty acids are activated for the coenzyme A (CoA) and used for the acylation of glycerol, which is pivotal to the synthesis of the triglycerides. In the first step of assembling triglycerides, glycerol-3-phosphate (G-3-P) is acylated via the specific acyltransferase of the glycerol-3-phosphate (glycerol-3-phosphate acyltransferase or SCT1) in order to yield lysophosphatidic acid, which is then acylated via the specific acyltrasferase of the lysophosphatidic acid (phosphatidic acid acyltranferase or SLC1) in order to yield phosphatidic acid (PA). The latter is then dephosphorylated via a specific phosphohydrolase of the phosphatidic acid (phosphatidic acid phosphohydrolase (PAP)) in order to release diacylglycerol (DAG). In the final step, the diacylglycerol is acylated either by diacylglycerol acyltransferase or by phospholipid diacylglycerol acyltransferase, in order to produce triglycerides.

In particular, it is particularly advantageous to use a substrate cheap and widely available such as sucrose. In this regard, it has been shown that overexpression of the S. cerevisiae gene SUC2 encoding invertase in an oleaginous yeast such as Y. lipolytica enables the said yeast to use sucrose by breaking it down into fructose and glucose (Lazar et al., 2013). Actually, when SUC2 is introduced in a strain overexpressing ylHXK1, the resulting strain grown on sucrose gives the largest overall amounts of lipids, whereas the same strain grown on glucose or fructose produces significantly lower concentrations. Thus sucrose turns out to be a better substrate for lipid production for such a strain than either of its building blocks, glucose or fructose.

An embodiment of the invention thus relates to an oleaginous yeast strain, e.g. a strain of Y. lipolytica, overexpressing a hexokinase such as ylHXK1 and the S. cerevisiae SUC2, said strain being capable of accumulating lipids.

The strain of the invention can be further improved by increasing the efficiency of the transport of hexose, and particularly fructose, in the cell. Indeed, formation of higher amounts of fructose-6-phosphate may be achieved either by increasing the activity of hexokinase and/or by increasing the amount of fructose (i.e. the substrate of hexokinase) in the cell.

In yeast, the uptake of hexoses, such as glucose and fructose, is mediated by specific hexose transporters that belong to a superfamily of monosaccharide facilitators. The proteins belonging to this family exhibit strong structural conservation although they may share little sequence similarity.

In S. cerevisiae, the HXT family encodes 20 different hexose transporters. Most of these transporters operate by facilitated diffusion (Leandro et al., 2009). The various hexose transporters differ considerably in substrate specificity and affinity. In a series of experiments with mutant yeast strains expressing only one of the genes HXT1 through HXT7, it was shown that Hxt1 and Hxt3 are low-affinity transporters (K_{M}≈50-100 mM hexose), Hxt4 is moderately low, and Hxt2, Hxt6 and Hxt7 are high affinity transporters (K_{M}≈1-4 mM hexose), regardless of the culture conditions of these mutants (0.1% or 5% glucose) (Reifenberger et al., 1995). Most hexose carriers display a stronger affinity for glucose compared to fructose. This is especially the case for the low affinity carriers Hxt1 (K_{M}≈110 mM for glucose versus >300 mM for fructose) and Hxt3 (K_{M}≈65 mM for glucose versus 125 mM for fructose).

In a preferred embodiment, the invention thus relates to an oleaginous yeast strain overexpressing a hexokinase, notably ylHXK1, and overexpressing a hexose transporter, said yeast strain being capable of accumulating lipids. More preferably, this strain further overexpresses SUC2.

A “transporter” refers to a protein responsible for transfer of the molecule to be transported from the extracellular culture medium into the cell or vice versa, i.e. effecting its passage, e.g. diffusion, across the plasma membrane. A “hexose transporter” thus refers to a transporter which may be a naturally occurring protein or a functionally equivalent variant as described herein, which is able to transport a saccharide as described above. A “hexose transporter” according to the invention is for example any one of the Hxt1, Hxt2, Hxt3, Hxt4, Hxt5, Hxt6, or Hxt7 proteins of budding yeast, or their homologues in other yeasts.

Advantageously, low-affinity hexose transporters are used (k_{M}=20-100 mM) in the oleaginous yeast of the invention. Preferably Hxt1 and/or Hxt3 genes are used.

By “Hxt1”, it is herein referred to a low-affinity transporter for hexoses having higher affinity for glucose than for fructose and represented by e.g. the protein having the amino acid sequence as in NP_011962 and encoded by the gene HXT1 (YHR094C) which has a nucleotide sequence as in NM_001179224.

By “Hxt3”, it is herein referred to a low-affinity transporter for hexoses having higher affinity for glucose than for fructose and represented by e.g. the protein having the amino acid sequence as in NP_010632 and encoded by the gene HXT3 (YDR345C) which has a nucleotide sequence as in NM_001180653. Hxt3 mutants

The present inventors have now identified new yeast hexose transporters. More specifically, the inventors have now identified 24 new genes, each of which encodes a putative Y. lipolytica sugar transporter. These genes are listed in Table 1.

TABLE 1
Sugar transporters in Y. lipolytica in E150 strain

| | Protein | Gene | |
|---|---|---|---|
| | systematic | usual | |
| N° | name | name | |
| 1 | A01958 | YSP1 | Yarrowia lipolytica putative sugar transporter |
| 2 | A08998 | YSP2 | Yarrowia lipolytica putative sugar transporter |
| 3 | A14212 | YSP3 | Yarrowia lipolytica putative sugar transporter |
| 4 | B00396 | YSP4 | Yarrowia lipolytica putative sugar transporter |
| 5 | B01342 | YHT5 | Yarrowia lipolytica hexose transporterYht5 |
| 6 | B06391 | YHT6 | Yarrowia lipolytica hexose transporterYht6 |
| 7 | B17138 | YSP7 | Yarrowia lipolytica putative sugar transporter |
| 8 | B21230 | YSP8 | Yarrowia lipolytica putative sugar transporter |
| 9 | C04686 | YSP9 | Yarrowia lipolytica putative sugar transporter, |
| | | | pseudogene |
| 10 | C04730 | YSP10 | Yarrowia lipolytica putative sugar transporter |
| 11 | C06424 | YHT1 | Yarrowia lipolytica hexose transporterYht1 |
| 12 | C08943 | YHT2 | Yarrowia lipolytica hexose transporterYht2 |
| 13 | C16522 | YSP13 | Yarrowia lipolytica putative sugar transporter |
| 14 | D00132 | YSP14 | Yarrowia lipolytica putative sugar transporter |
| 15 | D00363 | YSP15 | Yarrowia lipolytica putative sugar transporter |
| 16 | D01111 | YSP16 | Yarrowia lipolytica putative sugar transporter |
| 17 | D18876 | YSP17 | Yarrowia lipolytica putative sugar transporter |
| 18 | E20427 | YSP18 | Yarrowia lipolytica putative sugar transporter |
| 19 | E23287 | YHT4 | Yarrowia lipolytica hexose transporterYht4 |
| 20 | F06776 | YSP20 | Yarrowia lipolytica putative sugar transporter |
| 21 | F18084 | YSP21 | Yarrowia lipolytica putative sugar transporter |
| 22 | F19184 | YHT3 | Yarrowia lipolytica hexose transporterYht3 |
| 23 | F23903 | YSP23 | Yarrowia lipolytica putative sugar transporter |
| 24 | F25553 | YSP24 | Yarrowia lipolytica putative sugar transporter |

YHT; Yarrowia hexose transporter;
YSP; Yarrowia sugar porter;
The YALI proteins names are simplified for clarification; i.e. the annotation of YALI0A01958p is indicated as A01958.

In a specific embodiment of the invention, the oleaginous yeast strain overexpressing a hexokinase, notably ylHXK1, overexpresses a hexose transporter selected in the list of Table 1, said yeast strain being capable of accumulating lipids. More preferably, this strain further overexpresses SUC2.

In particular, the inventors have identified 6 Y. lipolytica hexose transporters, designated Yht1, Yht2, Yht3, Yht4, Yht5, and Yht6 (see Table 1). Thus, the hexose transporter expressed by the oleaginous yeast of the invention is preferably selected from the group of Yht1-6.

The hexose transporters of the invention are functional in Y. lipolytica since deletion thereof, either individually or in combination, leads to defects in carbon source utilization. For example, strain deleted for YHT1 is unable to grow on fructose 0.1%; strains deleted for both YHT1 and YHT4, or YHT1-flare unable to grow on glucose, mannose and fructose.

These proteins, Yht1 to Yht5 are capable of restoring growth on glucose and/or on fructose to a budding yeast mutant entirely devoid of the Hxt1-7 transporters, while Yht6 is capable of restoring growth only on mannose and galactose. In particular, expression of YHT3 enables S. cerevisiae to utilize glucose and fructose at the same time, whereas a yeast cell expressing YHT1 and YHT4 imports fructose only when glucose concentration is low (YHT1) or when glucose has been fully consumed (YHT4). On the other hand, expression of YHT5 only allows growth of the host cell on glucose, but not on fructose, while expression of YHT2 allows growth on fructose but not on glucose.

Expression of YHT1, YHT3 or YHT4 in a Yarrowia lipolytica yht1-4 quadruple mutant restores the capacity of the cell to utilize sugars. In particular, expression of YHT3 or YHT1 enables Y. lipolytica to utilize glucose and fructose at the same time, whereas a yeast cell expressing YHT4 only imports fructose after glucose has been fully consumed.

In a specific aspect, the invention thus relates to a Y. lipolytica Yht1 protein, said protein having the sequence of SEQ ID NO: 14. The Yht1 protein is a Y. lipolytica homolog of the budding yeast Hxt1. It should be emphasized that the protein of SEQ ID NO: 14 is the protein encoded by the YHT1 gene present in reference strain E150, whose genome was completely sequenced (Dujon et al., 2004). However, it is well known in this field that Y. lipolytica strains show some degree of polymorphism. In the present case, the inventors have shown that Yht1 proteins isolated from the H222 strain differ from the one of E150 and W29. Thus the invention also relates to a Y. lipolytica Yht1 protein from the H222 or the W29 strain, said protein having the sequence of SEQ ID NO: 15 or SEQ ID NO: 16, respectively.

In another aspect, the invention relates to a Y. lipolytica Yht2 protein, said protein being isolated from the E150, the H222, or the W29 strain, and having the sequence represented by SEQ ID NO: 17, SEQ ID NO: 18, or SEQ ID NO: 19, respectively.

In another aspect, the invention relates to a Y. lipolytica Yht4 protein, said protein being isolated from the E150, the H222, or the W29 strain, and having the sequence represented by SEQ ID NO: 26, SEQ ID NO: 27, or SEQ ID NO: 28, respectively.

In another aspect, the invention relates to a Y. lipolytica Yht5 protein, said protein being isolated from the E150, the H222, or the W29 strain, and having the sequence represented by SEQ ID NO: 7, SEQ ID NO: 8, or SEQ ID NO: 9, respectively.

In yet another aspect, the invention relates to a Y. lipolytica Yht6 protein from the E150 strain, having the sequence represented by SEQ ID NO: 10.

In another aspect, the invention relates to a Y. lipolytica Yht3 protein, said protein being isolated from the E150, the H222, or the W29 strain, and having the sequence represented by SEQ ID NO: 31, SEQ ID NO: 32, or SEQ ID NO: 33, respectively. The inventors have shown that YHT3 from H222 is the most efficient in rescuing a budding yeast strain lacking all Hxt1-7 transporters. Preferably, the Yht3 protein of the invention has the sequence SEQ ID NO: 32.

DNA sequences derived from these genes by substitution, deletion or addition of one or more nucleotides in such a way that the DNA sequence still encodes a protein capable of transporting hexose(s) are considered to be a part of this invention. In a specific example, the Yht1 protein of the invention comprises a valine at position 162 and has the sequence represented by SEQ ID NO: 36. In a preferred embodiment, the Yht3 protein of the invention comprises a valine at position 181 and has the sequence represented by SEQ ID NO: 37. Expression in Y. lipolytica of this specific Yht3-I181V protein results in improved assimilation of both glucose and fructose.

Thus, in a preferred embodiment, the yeast strain of the invention further overexpresses YHT1 or YHT3, preferably YHT3, more preferably YHT3-I181V. In addition, this strain may overexpress SUC2.

It will be appreciated that the yeast strain of the invention can be further modified by introducing additional mutations therein, in order to improve the amount and/or the nature of the lipids produced.

The present inventors previously constructed yeast strains which yield very high amounts of lipids. For example, the knock-out of the gene GUT2 results in an increased accumulation of lipids in yeasts, particularly in Y. lipolytica (WO 2010/004141; Beopoulous et al., 2008). The gene GUT2 encodes the isoform Gut2p of the glycerol-3-phosphate dehydrogenase, which catalyzes the oxidation reaction of the glycerol-3-phosphate into DHAP.

Thus in a preferred embodiment, the yeast strain of the invention does not express Gut2p.

On the other hand, the inventors have shown that it is possible to obtain an accumulation of lipids by overexpressing the gene GPD1 in yeasts in which the beta-oxidation of the fatty acids is deficient (WO 2012/001144). GPD1 encodes the glycerol-3-phosphate dehydrogenase catalyzes the synthesis reaction of glycerol-3-phosphate from DHAP.

Thus in another preferred embodiment, the yeast strain of the invention overexpresses GPD1 and is deficient for beta-oxidation of the fatty acids.

The beta-oxidation involves four successive reactions which occur during degradation pathway of fatty acids and involved an acyl-CoA oxidase which six isoforms are encoded by six POX genes, a multifunctional enzyme encoded by the gene MFE1 and a 3-ketoacyl-CoA thiolase encoded by the POT1 gene (Table 2). Beta-oxidation in yeast takes place exclusively in the peroxisome, a cytoplasmic organelle whose biogenesis is controlled by the PEX genes (see Table 3). When the peroxisome is not properly assembled or when it is not functional, the fatty acids are not properly degraded (WO 2006/064131; Thevenieau et al., 2007).

In general, mutations affecting the beta-oxidation according to the invention are loss-of-function mutations that result in a strong reduction or even in a complete absence of beta oxidation. The loss-of-function mutations of the invention may be point mutations, insertions, deletions (total or partial), gene replacement or any other molecular cause that leads to a substantial decrease in beta-oxidation.

Yeast strains in which the beta-oxidation of fatty acids is deficient according to the present invention include all strains carrying at least one loss-of-function mutation in at least one gene encoding an enzyme directly involved in beta-oxidation. These strains also encompass all the strains that carry at least one loss-of-function mutation that affects beta-oxidation only indirectly, including through the biogenesis and function of peroxisomes. It is understood that the strains according to the invention also include all strains carrying combinations of the mutations described above. For example, are encompassed within the scope of the present invention, the strains that carry at least one loss-of-function mutation which affects beta-oxidation directly and at least one loss-of-function mutation which affects beta-oxidation only indirectly.

According to a preferred aspect of the invention, the strains deficient in the beta-oxidation of fatty acids include any strain carrying a loss-of-function mutation in the PEX genes listed in Table 3. According to another preferred aspect of the invention, strains deficient in beta-oxidation of fatty acids include strains carrying at least one loss-of-function mutation in one of the following genes: POX1, POX2, POX3, POX4, POX5, POX6, MFE1, and POT1. More preferably, the strains according to the invention comprise at least a loss-of-function mutation in at least one gene POX1, POX2, POX3, POX4, POX5 and POX6. Even more preferably, the strains according to the invention include mutations in each of the genes POX1, POX2, POX3, POX4, POX5 and POX6.

According to a particular embodiment, the invention relates to an oleaginous yeast strain, notably a strain of Y. lipolytica, which overexpresses a hexokinase gene such as ylHXK1, and which also overexpresses the GPD1 gene and comprises at least one loss-of-function mutation in at least one gene involved in the beta-oxidation of fatty acids, said yeast strain being able to accumulate lipid. Advantageously, said yeast strain comprises at least a loss-of-function mutation in at least one of the genes selected from PEX, POX, and MFE1 POT1 gene. More preferably, the POX genes are partially (POX2 to POX5) or totally (POX1 to POX6) inactivated in the mutant strain of the invention, said yeast strain being able to accumulate lipid.

In addition to the aforementioned loss-of-function mutations, which lead to an impairment of beta-oxidation, the yeast strain according to the invention may comprise one or more additional mutations in at least one gene encoding an enzyme involved in the metabolism of fatty acids. These additional mutations may further increase the capacity of the strain to accumulate lipids. Alternatively, they may alter the profile of stored fatty acids.

For example, the genes encode TGL3 and TGL4 lipases involved in the remobilisation of triglycerides (Kurat et al., 2006; WO 2012/001144). The present inventors showed that inactivation of TLG3 and/or TLG4 leads to higher lipid accumulation (Dulermo et al., 2013). The invention therefore also relates to a yeast strain, preferably a strain of oleaginous yeast, particularly a strain of Y. lipolytica, overexpressing the GPD1 gene, and deficient in the beta-oxidation of fatty acids, said strain overexpressing a hexokinase gene such as ylHXK1 and being capable of accumulating lipids, wherein said strain further carries at least one loss-of-function mutation in TLG3 or TLG4. Preferably, the said strain carries mutations in both genes.

In Y. lipolytica, the major acyl-CoA:diacylglycerol acyltransferase activity is encoded by the ylDGA2 gene (YALI0D07986g) (Beopoulos et al., 2012). This activity is responsible for the formation of TAGs by catalyzing the acyl-CoA-dependent acylation of sn-1,2-diacylglycerol, a rate-limiting step in the formation of TAGs. Hence, the invention also relates to a strain of oleaginous yeast, such as Y. lipolytica, which overexpresses a hexokinase gene, e.g. ylHXK1 and is capable of accumulating lipids, said strain further overexpressing the ylDGA2 gene. (Completer avec DGA1 et LRO1?).

It has also been shown that inactivation of the ylFAD2 gene (YALI0B10153g), which encodes a Δ12 fatty acid desaturase, increases the proportion of fatty acid C18:1 (WO 2005/047485). The present invention thus also provides a strain of oleaginous yeast, preferably a strain of Y. lipolytica, overexpressing a hexokinase gene such as ylHXK1 and being capable of accumulating lipids, said strain further comprising an inactivated gene YALI0B10153g.

In another embodiment, the yeast strain of the invention further comprises a gene whose expression is used to modify the fatty acid profile of said strain. Indeed, it was previously shown that the ectopic expression of certain genes encoding desaturases can alter the polyunsaturated fatty acids pattern in a yeast strain, notably in Y. lipolytica. Thus the expression of a Δ12 fatty acid desaturase yield increased quantities of C18:2 fatty acids (WO 2005/047485). Similarly, the expression of a Δ8 desaturase or a Δ15 desaturase leads to a change of the pattern of fatty acids in Y. lipolytica (WO 2005/047480, WO 2006/012325). The invention therefore also relates to a yeast strain, preferably a strain of oleaginous yeast, particularly a strain of Y. lipolytica, overexpressing a hexokinase gene such as ylHXK1 and being capable of accumulating lipids, said strain further expressing a gene encoding an enzyme selected from Δ8-desaturase, Δ12-desaturase and Δ15 desaturase. Preferably, the enzyme is a Δ12 desaturase. Still more preferably, the gene encoding said Δ12 desaturase is the Y. lipolytica gene whose accession number is YALI0B10153g.

It will be immediately clear to the person of skills in the art that the mutations described above can be combined in order to create genetic backgrounds wherein overexpression of the hexokinase will result in an even greater accumulation of lipids. For example, it may be advantageous to delete all six POX genes while overexpressing at the same time the ylDGA2 gene. Alternatively, the deletion of POX1-6 may be combined with the inactivation of the TLG3 and/or TLG4 genes. Of course, the POX1-6 deletion may be constructed in a strain wherein the TLG3 and/or TLG4 genes are deleted and the ylDGA2 gene is overexpressed. More preferably, these strains overexpress the GPD1 gene as well.

Thus the invention also provides an oleaginous yeast strain comprising any combination of the mutations described above, said strain further overexpressing the ylHXK1 gene and being capable of accumulating lipids. Preferably, the yeast strain of the invention further overexpresses YHT1 or YHT3, preferably YHT3, more preferably YHT3-I181V. In addition, this strain may overexpress SUC2.

The invention also relates to a method for constructing a yeast strain which is capable of accumulating lipids, wherein the said method comprises the step of transforming the yeast strain with a polynucleotide allowing the overexpression of a hexokinase gene.

In a preferred embodiment, the yeast is an oleaginous yeast. In a more preferred embodiment, the yeast is R. glutinis, R. toluroides or Y. lipolytica. In a further more preferred embodiment, the yeast is Y. lipolytica.

In another preferred embodiment, the hexokinase gene is the gene ylHXK1. The ylHXK1 gene can be overexpressed by any manner known to a person skilled in the art.

To accomplish this, each copy of the ylHXK1 open reading frame is placed under the control of appropriate regulatory sequences. Said regulatory sequences include promoter sequences placed upstream (5′) from the ylHXK1 open reading frame, and terminator sequences placed downstream (3′) from the ylHXK1 open reading frame.

The promoter and terminator sequences used preferably belong to different genes, so as to minimize the risks of undesirable recombination in the genome of the Yarrowia strain.

Such promoter sequences are well known to a person skilled in the art and can, in particular, correspond to inducible and constitutive promoters. As examples of promoters that can be used in the method of the invention, reference can be made in particular to the promoter of a Y. lipolytica gene that is strongly repressed by glucose and that can be induced by fatty acids or triglycerides, such as the POX2 promoter of the acyl-CoA oxidase gene of Y. lipolytica and the promoter of the LIP2 gene described in PCT application WO 01/83773. It is also possible to use the FBA/gene promoter of the fructose-bisphosphate aldolase gene (US 2005/0019297), the GPM promoter of the phosphogylcerate mutase gene (WO 2006/0019297), the YAT1 gene promoter of the ammonium transporter gene (US 2006/0094102 A1), the GPAT gene promoter of the glycerol-3-phosphate O-acyltransferase gene (US 2006/0057690 A1), the TEF gene promoter (Muller et al., 1998; US 2001/6265185), the hp4d hybrid promoter (WO 96/41889) or even the XPR2 hybrid promoters described in Mazdak et al. (2000).

Such terminator sequences are likewise well-known to a person skilled in the art, and, as examples of terminator sequences that can be used in the method according to the invention, reference can be made to terminator sequence of the PGK1 gene, and the terminator sequence of the LIP2 gene described in PCT application WO 01/83773.

The overexpression of ylHXK1 can be obtained by replacing the sequences controlling the expression of ylHXK1 by regulatory sequences enabling stronger expression, such as those described above. A person skilled in the art can thus replace the copy of the ylHXK1 gene in the genome, as well as the specific regulatory sequences thereof, by transforming the mutant strain of yeast with a linear polynucleotide including the ylHXK1 open reading frame under the control of regulatory sequences such as those described above. Said polynucleotide is advantageously flanked by sequences that are homologues of sequences situated on each side of the chromosomal ylHXK1 gene. Insofar as this recombination event is rare, selection markers are inserted between the sequences ensuring recombination so that, after transformation, it is possible to isolate the cells where integration of the fragment occurred, by highlighting the corresponding markers.

The overexpression of ylHXK1 is obtained by introducing into the strain of yeast according to the invention supernumerary copies of the ylHXK1 gene under the control of regulatory sequences such as those described above. Said additional copies of ylHXK1 can be carried by an episomal vector, i.e., one capable of replicating in the yeast.

Said copies are preferably carried by an integrative vector, i.e., being integrated at a specific location in the genome of the yeast (Mazdak et al., 2004). In this case, the polynucleotide comprising the GPD1 gene under the control of regulatory regions is integrated by targeted integration.

Targeted integration of a gene in the yeast genome is a technique frequently used in molecular biology. In this technique, a DNA fragment is cloned in an integrative vector introduced into a cell being transformed, which DNA fragment is then integrated by homologous recombination in a targeted region of the recipient genome (Orr-Weaver et al., 1981). Such transformation methods are well known to a person skilled in the art and are described, in particular, in Ito et al. (1983), in Klebe et al. (1983) and in Gysler et al. (1990). Insofar as this recombination event is rare, selection markers are inserted between the sequences ensuring recombination so that, after transformation, it is possible to isolate the cells where integration of the fragment occurred, by highlighting the corresponding markers.

They can also be carried by PCR fragments the ends of which have homology with a specific locus of the yeast, thus enabling said copies to be integrated into the genome of the yeast by homologous recombination.

Any transfer method known to a person skilled in the art can be used to introduce the invalidation cassette 1 into the yeast strain. Preferably, use can be made of the lithium acetate and polyethylene glycol method (Gaillardin, 1987; Le Dall et al., 1994).

According to the invention, it is possible to use any selection method known in the prior art, which is compatible with the gene (or genes) used, any strain expressing the selected marker gene potentially being a strain of yeast defective with regard to the GUT2, URA3 or LEU2 gene.

Selection markers enabling auxotrophy complementation, likewise commonly called auxotrophy markers, are well-known to a person skilled in the art.

The URA3 selection marker is well-known to a person skilled in the art. More specifically, a strain of Y. lipolytica, the URA3 gene of which (YALIOE26719g), encodes for the orotidine-5′-phosphate decarboxylase, is inactivated (e.g., by deletion), will not be capable of growing in a medium not supplemented with uracil. Integration of the URA3 selection marker into this strain of Y. lipolytica will then enable the growth of this strain to be restored in a uracil-free medium.

The LEU2 selection marker, described in particular in U.S. Pat. No. 4,937,189, is likewise well-known to a person skilled in the art. More specifically, a strain of Y. lipolytica, of which the LEU2 gene (YALIOE26719g) encodes for the β-isopropylmalate dehydrogenase, is inactivated (e.g., by deletion), and will not be capable of growing in a medium not supplemented with leucine. As previously, integration of the LEU2 selection marker will then enable the growth of this strain to be restored in a medium not supplemented with leucine.

The ADE2 selection marker is likewise well-known to a person skilled in the art, in the field of yeast transformation. A strain of Yarrowia, of which the ADE2 gene (YALIOB23188g) encodes for the phosphoriboxylaminoimidazole carboxylase, is inactivated, and will not be capable of growing in a medium not supplemented with adenine. Here again, integration of the ADE2 selection marker in this strain of Y. lipolytica will then allow one to restore the growth of this strain on a medium not supplemented with adenine.

In a preferred embodiment of the invention, the method for constructing a yeast strain capable of accumulating lipids may comprise a further step of introducing at least one additional mutation affecting lipid synthesis. Such mutation may affect preferably one of the genes listed above, such as e.g. at least one of the genes controlling beta-oxidation, the TLG3 and TLG4 genes, GUT2, or YALI0B10153g. In a further preferred embodiment, this step is repeated so that different mutations are introduced in the same strain.

In another preferred embodiment of the invention, the method for constructing a yeast strain capable of accumulating lipids may comprise a further step of introducing at least one additional polynucleotide enabling the overexpression of another gene regulating lipid synthesis. Preferably, the said gene is one of the genes described above, such as e.g. YHT1, YHT3 (notably YHT3-I181V), SUC2, GPD1, or ylDGA2. In a further preferred embodiment, this step is repeated so that different polynucleotides carrying distinct genes are introduced in the same strain.

In yet another further preferred embodiment, the method of the invention comprises a further step of introducing at least one additional mutation affecting lipid synthesis and a further step of introducing at least one additional polynucleotide enabling the overexpression of another gene regulating lipid synthesis. Each of these steps can be repeated in order to introduce different mutations and/or different polynucleotides carrying distinct genes in the same strain. The method of the invention thus generates oleaginous yeast strains, notably strains of Y. lipolytica, carrying all the possible combinations of mutations and/or polynucleotides described above.

These further steps may be carried out either simultaneously or consecutively with the step of transforming the yeast strain with a polynucleotide allowing the overexpression of the gene ylHXK1. If these steps are carried out one after the other, the order in which they are performed does not matter.

Overexpression of the said genes can be achieved as described above.

The prior art also teaches various methods that allow the construction of an oleaginous yeast stain, especially a Y. lipolytica strain, wherein a gene is inactivated. In particular, the POP IN/POP OUT method has been used in yeast, especially in Y. lipolytica for deleting the genes LEU2, URA3 and XPR2 as described in the review of G. Barth et al. (1996). According to this method, a vector comprising an inactivated allele of a gene of interest is first integrated at the corresponding chromosomal locus. This creates a duplication with the wild-type and mutant copies of the gene flanking the plasmid sequences. After the excision of said vector is induced, recombinant clones that have eliminated the wild-type gene and retained the mutated gene can be identified.

Preferably, the method according to the invention results in the inactivation of the gene of interest.

By “inactivation” or “knock-out” of a gene of interest (both terms as used herein are synonymous and therefore have the same meaning), it is herein referred to any method that results in the absence of expression of the protein encoded by said native gene of interest, by modifying the nucleotide chain constituting said gene in such a way that, even if its translation were to be effective, it would not lead to the expression of the native protein coded by the wild type gene of interest.

Preferably, a method leading to a total suppression of the expression of the gene of interest is used. This can be achieved by a total deletion of the gene of interest in a partial deletion of the gene of interest, by insertion of one or more nucleotides in said gene of interest, said method making the gene of interest non-functional (or inactivated gene of interest invalidated), at least not encoding a protein having the properties of said native protein.

Thus, a yeast strain not expressing the gene of interest is obtained by the method above, which is called in this text “strain defective in the gene of interest.”

The skilled person can also use the SEP method (Maftahi et al., 1996) which was adapted in Y. lipolytica for the successive disruption of all 6 POX genes (Wang et al., 1999). This method is quicker, but still requires the use of a counter-selection marker. Advantageously according to the invention, the SEP/Cre method developed by Fickers et al. (2003) and described in international patent application WO 2006/064131 is used. This is a quick method that does not require the use of a counter-selection marker.

This method comprises the steps of:
1) selecting the gene of interest that is to be deleted,
2) constructing a disruption cassette by PCR (“Polymerase Chain Reaction) or by cloning,
3) introducing a selectable marker flanked by identical recombination sequences (preferably the loxP and/or loxR sequences or derivatives thereof), thus permitting elimination of the marker (preferably a loxP-type sequence that allows recombination under the action of Cre recombinase) by recombination between said sequences,
4) selecting the strains carrying a deletion in the gene of interest (transformation and selection of transformants) and verifying the deletion,
5) transforming with a vector allowing the expression of the recombinase (advantageously the Cre recombinase which allows recombination of the loxP/loxR sequences and the removal of the marker)
6) isolating a clone wherein the gene of interest is deleted and the recombinase expression plasmid lost.

The insertion cassette of step 2 comprises a gene encoding a selection marker (selection gene), said gene being preferably flanked by the promoter and terminator regions of the gene of interest, so as to allow the replacement of the whole coding region of the gene of interest by homologous recombination. According to a particular embodiment, the selection gene too is flanked by one or more recombination sequences, said sequences enabling elimination of the gene encoding the selectable marker by recombination between them. Preferably the recombination sequences are loxP and/or loxR sequences or derivatives thereof, said derivatives having retained the activity of the original recombination sequences. Preferably, at this stage, the gene encoding the selectable marker may be flanked by loxP-type sequences which, under the action of the Cre recombinase, recombine between them, giving rise to a plasmid carrying the selection marker gene.

The introduction of the knock-out cassette in the recipient yeast strain in step 3 can be carried out by any technique known to the skilled person. As noted above, the said person will refer to G. Barth et al. (1996).

Transformants expressing the selection marker are selected in step 4. The presence of the marker can be verified by any conventional method known to the person of skills in the art, such as PCR or Southern blot hybridization.

In step 5, a plasmid allowing expression of a recombinase is introduced into a transformant selected in the previous step. Preferably, the plasmid carries a gene encoding the Cre recombinase (Sauer, 1987) which induces recombination of loxP/loxR sequences and the removal of the marker. This technique is commonly used by those skilled in the art seeking to excise specific integrated sequence (Hoess and Abremski, 1984).

Step 6 is a standard step of selecting a clone wherein the selection gene has been excised, said clone thus having a phenotype of absence of the selection marker.

In a specific embodiment of the invention, at least one gene controlling beta-oxidation is inactivated. As noted above, these genes are both the MFE1, POT1, and POX genes (Table 2), and the PEX genes (Table 3).

TABLE 2
Genes involved in fatty acids metabolism in yeast, notably in
Y. lipolytica. The sequences are available through their names
or their accession numbers at http://gryc.inra.fr/
(formerly www.genolevures.org).

| Gene | Name | N° EC | Function |
|---|---|---|---|
| GUT1 | YALI0F00484g | EC 2.7.1.30 | Glycerol kinase |
| GPD1 | YALI0B02948g | EC 1.1.1.18 | Glycerol-3-phosphate |
| | | | dehydrogenase (NAD(+)) |
| GUT2 | YALI0B13970g | EC 1.1.99.5 | Glycerol-3-phosphate |
| | | | dehydrogenase |
| SCT1 | YALI0C00209g | EC 2.3.1.15 | Glycerol-3-phosphate |
| | | | acyltransferase |
| SLC1 | YALI0E18964g | EC 2.3.1.51 | 1-acyl-sn-glycerol-3- |
| | | | phosphate acyltransferase |
| DGA1 | YALI0E32769g | EC 2.3.1.20 | Diacylglycerol acyltransferase |
| LRO1 | YALI0E16797g | EC 2.3.1.158 | Phospholipid:diacylglycerol |
| | | | acyltransferase |
| TGL3 | YALI0D17534g | EC 3.1.1.3 | Triacylglycerol lipase |
| TGL4 | YALI0F10010g | EC 3.1.1.3 | Triacylglycerol lipase |
| ARE1 | YALI0F06578g | EC 2.3.1.26 | Acyl-CoA:sterol |
| | | | acyltransferase |
| DGA2 | YALI0D07986g | EC 2.3.1.20 | Diacylglycerol acyltransferase |
| TGL1 | YALI0E32035g | EC 3.1.1.13 | Cholesterol esterase |
| POX1 | YALI0E32835g | EC 6.2.1.3 | Acyl-coenzyme A oxidase |
| POX2 | YALI0F10857g | EC 6.2.1.3 | Acyl-coenzyme A oxidase |
| POX3 | YALI0D24750g | EC 6.2.1.3 | Acyl-coenzyme A oxidase |
| POX4 | YALI0E27654g | EC 6.2.1.3 | Acyl-coenzyme A oxidase |
| POX5 | YALI0C23859g | EC 6.2.1.3 | Acyl-coenzyme A oxidase |
| POX6 | YALI0E06567g | EC 6.2.1.3 | Acyl-coenzyme A oxidase |
| MFE1 | YALI0E15378g | EC 4.2.1.74 | Multi-functional beta |
| | | | oxidation protein |
| POT1 | YALI018568g | EC 2.3.1.16 | Peroxisomal Oxoacyl Thiolase |

TABLE 3
Genes involved in peroxisome metabolism in yeast, notably in
Y. lipolytica. The sequences are available through their names or
their accession numbers at http://gryc.inra.fr/
(formerly www.genolevures.org).

| | Accession | Accession | |
|---|---|---|---|
| | number | number | |
| Gene | S. cerevisiae | Y. lipolytica | Function |
| PEX1 | YKL197c | YALI0C15356g | AAA-peroxin |
| PEX2 | YJL210W | YALI0F01012g | RING-finger peroxin |
| | | | which functions in |
| | | | peroxisomal matrix |
| | | | protein import |
| PEX3 | YDR329c | YALI0F22539g | Peroxisomal membrane |
| | | | protein (PMP) |
| PEX4 | YGR133w | YALI0E04620g | Peroxisomal ubiquitin |
| | | | conjugating enzyme |
| PEX5 | YDR244w | YALI0F28457g | Peroxisomal membrane |
| | | | signal receptor |
| PEX6 | YNL329c | YALI0C18689g | AAA-peroxin |
| PEX7 | YDR142c | YALI0F18480g | Peroxisomal signal receptor |
| PEX8 | YGR077c | / | Intraperoxisomal organizer |
| | | | of the peroxisomal import |
| | | | machinery |
| PEX9 | / | YALI0E14729g | Peroxisomal integral |
| | | | membrane protein |
| PEX10 | YDR265w | YALI0C01023g | Peroxisomal membrane |
| | | | E3 ubiquitin ligase |
| PEX11 | YOL147c | YALI0C04092g | Peroxisomal membrane |
| | | | protein |
| PEX12 | YMR026c | YALI0D26642g | C3HC4-type RING-finger |
| | | | peroxisomal membrane |
| | | | peroxin |
| PEX13 | YLR191w | YALI0C05775g | Integral peroxisomal |
| | | | membrane |
| PEX14 | YGL153w | YALI0E9405g | Peroxisomal membrane |
| | | | peroxin |
| PEX15 | YOL044w | / | Phosphorylated tail- |
| | | | anchored type II integral |
| | | | peroxisomal membrane |
| | | | protein |
| PEX16 | / | YALI0E16599g | Intraperoxisomal peripheral |
| | | | membrane peroxin |
| PEX17 | YNL214w | / | Peroxisomal membrane |
| | | | peroxin |
| PEX18 | YHR160c | / | Peroxin |
| PEX19 | YDL065c | YALI0B322660g | Chaperone and import |
| | | | receptor |
| PEX20 | / | YALI0E06831g | Peroxin |
| PEX21 | YGR239c | / | Peroxin |
| PEX22 | YAL055w | / | Putative peroxisomal |
| | | | membrane protein |
| PEX23 | PEX30 | YALI0D27302g | Integral peroxisomal |
| | (YLR324w) | | membrane peroxin |
| | PEX31 | | |
| | (YGR004w) | | |
| | PEX32 | | |
| | (Y8R168w) | | |
| PEX25 | YPL112c | YALI0D05005g | Peripheral peroxisomal |
| | | | membrane peroxin |
| PEX27 | YOR193w | / | Peripheral peroxisomal |
| | | | membrane protein |
| PEX28 | YHR150w | YALI0D11858g | Peroxisomal integral |
| | | YALI0F19580g | membrane peroxin |
| PEX29 | YDR479c | YALI0F19580g | Peroxisomal integral |
| | | | membrane peroxin |
| PEX30 | YLR324W | YALI0D27302g | Peroxisomal integral |
| | | | membrane protein |
| PEX31 | YGR004W | YALI0D27302g | Peroxisomal integral |
| | | | membrane protein |
| PEX32 | Y8R168w | YALI0D27302g | Peroxisomal integral |
| | | | membrane protein |

In this embodiment, the invention relates specifically to a method for obtaining a strain of an oleaginous yeast, notably a Y. lipolytica strain, which does not express a gene controlling beta-oxidation, wherein:
in a first step, an invalidation cassette is constructed, which includes the promoter and terminator sequences of said gene of oleaginous yeast, notably of Y. lipolytica, flanking a gene encoding a selection marker (selection gene), said selection gene itself being flanked on both sides of the sequence thereof by one (or more) recombination sequence(s), said recombination sequences enabling recombination there between, thus resulting in the elimination of said selection gene;
in a second step, said invalidation cassette obtained in step 1 is introduced into a strain of oleaginous strain of yeast, notably Y. lipolytica;
in a third step, a clone of yeast is selected among the strains of oleaginous yeast (notably Y. lipolytica) transformed in step 2, which is defective with regard to the gene of interest, said strain having the marker gene replaced by said gene of interest via two recombination events, thereby resulting in an inactivated gene;
in a fourth step, the invalidation of said gene in said strain of yeast selected in step 3 is verified.

According to a specific embodiment of the invention, the method may comprise two additional steps, namely:
a fifth step, during which said strain selected in step 4 is transformed using a vector enabling the expression of a recombinase, so as to eliminate the gene expressing the selection marker;
a sixth step during which a strain of yeast is isolated, which is defective with regard to the gene and which no longer expresses the marker gene.

The method for inactivating a beta-oxidation gene can then be repeated so as to inactivate another gene, if necessary. A person skilled in the art will thus be able to inactivate as many genes as necessary, by simply repeating the SEP gene inactivation method. Said person can thus construct the mutant strains of yeast described above, which comprise several inactivated genes.

According to the invention, an oleaginous yeast strain that is unable to carry out the beta-oxidation of lipids may advantageously be used, e.g., a strain that will not express the genes responsible for the beta-oxidation of lipids, such as the POX, MFE1 or POT1 genes, advantageously a strain not expressing the POX gene, at the very least the POX2, POX3, POX4 and POX5 genes, preferably the POX1, POX2, POX3, POX4, POX5 and POX6 genes, e.g., such as the strains described in international application WO 2006/064131 published on Jun. 22, 2006, preferably the strains:
MTLY37 (Leu⁺, Ura⁺; Δpox5, Δpox2, Δpox3, Δpox4::URA3),
MTLY40 (Leu⁺, Ura⁻; Δpox5-PT, Δpox2-PT, Δpox3-PT, pox4::URA3-41),
MTLY64 (Leu⁻, Ura⁻; Δpox5, Δpox2, Δpox3, Δpox4::URA3-41, LEU2::Hyg),
MTLY66 (Leu⁻, Ura⁻; Δpox5, Δpox2, Δpox3, Δpox4::URA3-41, Δleu2),
MTLY82 (Leu⁻, Ura⁻; Hyg⁻; Δpox5, Δpox2, Δpox3, Δpox4::URA3-41, Δleu2, Δpox1),
MTLY86 (Leu⁻, Ura⁻; Δpox5; Δpox2, Δpox3, Δpox4::URA3-41, Δpox1),
MTLY92 (Leu⁻, Ura⁻; Hyg⁺; Δpox5, Δpox2, Δpox3, Δpox4::URA3-41, Δleu2, Δpox1, pox6::Hyg),
MTLY95a (Leu⁻, Ura⁻; Δpox5, Δpox2, Δpox3, Δpox4::URA3-41, Δleu2, Δpox1, Δpox6)

In another aspect of the invention, a yeast strain such as those described in PCT application WO 2010/004141 published on Jan. 14, 2010 may be used. For example, the following strains may be used:
JMY1351 (Leu⁻, Ura⁺, Δpox5, Δpox2, Δpox3, Δpox4::URA3-41, Δleu1, Δpox1, Δpox6, Δgut2)
JMY1393 (Leu⁻, Ura⁻, Δpox5, Δpox2, Δpox3, Δpox4::URA3-41, Δpox1, Δpox6, Δgut2).

In yet another aspect of the invention, the strains described in WO 2012/001144, Beopoulos et al. (2008, 2012), Dulermo et al. (2013) and Wang et al (1999) may be used in the method of the invention.

The invention also relates to the use of a strain of oleaginous yeast, in particular Y. lipolytica, for synthesizing lipids, especially free fatty acids and triacylglycerols. In a preferred embodiment, the invention relates to the use of a strain of oleaginous yeast, in particular Y. lipolytica, which overexpresses ylHXK1, as described above, for synthesizing free fatty acids and triacylglycerols. In a more especially preferred embodiment, the strain which is used for producing lipids comprises additional mutations, such as the ones described above, which result in an increased lipid yield.

The present invention also relates to a lipid-synthesizing method in which:
in a first step, a strain of oleaginous yeast according to the invention is grown in an culture appropriate medium, and
in a second step, the lipids produced by the culture of the first step are harvested.

Preferably, the appropriate medium of the invention comprises fructose as a carbon source. More preferably, the carbon source in the said medium is sucrose.

In addition to the preceding arrangements, the present invention likewise includes other characteristics and advantages, which will emerge from the following examples and figures, and which must be considered as illustrating the invention without limiting the scope thereof.

### FIGURE LEGENDS

FIG. 1. Schematic representation of strain construction.

The JMY3501 strain was derived from JMY1233 (Beopoulos et al., 2008). (i) TGL4 was inactivated by introducing the disruption cassette tgl4::URA3ex from JMP1364 (Dulermo et al., 2013), which generated JMY2179. (ii) An excisable auxotrophic marker, URA3ex, was then excised from JMY2179 using JMP547 (Fickers et al., 2003), which generated JMY3122. (iii) JMY3501 was then obtained by successively introducing pTEF-DGA2-LEU2ex, from JMP1822, and pTEF-GPD1-URA3ex, from JMP1128 (Dulermo and Nicaud, 2011), into JMY3122. JMP1822 was obtained by replacing the URA3ex marker from JMP1132 (Beopoulos et al., 2008) with LEU2ex.

The JMY4086 strain was generated by successively introducing pTEF-YlHXK1-URA3ex, from JMP2103, and pTEF-SUC2-LEU2ex, from JMP2347, into JMY3820. JMY3820 corresponds to JMY3501, but is different in that the URA3ex and LEU2ex markers in the former have been rescued, as previously described (Fickers et al., 2003).

FIG. 2. Growth curves of different Y. lipolytica WT strains (A,B) and ylHXK1-overexpression transformants (C,D) grown in YNB medium with 10 g·L⁻¹ glucose (A,C) or 10 g·L⁻¹ fructose (B,D). WT strains were W29 ( . . . ), A-101 (—), and H222 (- -); growth was analyzed using a Biotek apparatus.

FIG. 3. Cell morphology of Y. lipolytica WT and ylHXK1-overexpression transformants. Images are of the WT French line W29 (A), Polish line A-101 (C), and German line H222 (E), as well as of their respective overexpression transformants (B, D, E, respectively). Images were taken after 120 h of growth in flasks in YNB fructose medium (carbon source 100 g·L⁻¹).

FIG. 4. Fatty acid production by Y. lipolytica W29 (□) and its ylHXK1-overexpression transformant (▪) in YNB fructose medium with different C/N molar ratios (A) and rich YP medium with different fructose concentrations (B). In red: the improvement in FA production (%; ratio of ylHXK1 to WT). Lipid content was analyzed after 120 h of culture or after complete fructose consumption. Different C/N ratios were obtained by increasing fructose concentration.

FIG. 5. Sucrose (♦), glucose (▪), fructose (▴), CA (), dry biomass (×), and FA (∘) concentration during Y. lipolytica Y4086 (A) and Y3501 (B) growth in YNB medium with sucrose over the 96 h of culture in the bioreactor.

FIG. 6. Sugar utilization by Y. lipolytica WT (♦) and ylHXK1-overexpressing (▪) strains in YNB medium containing 100 g·L⁻¹ glucose or 100 g·L⁻¹ fructose over 120 h of growth in flasks. Strains represented are W29 (A,B), A-101 (C,D), and H222 (E,F).

FIG. 7. Fatty acid production by Y. lipolytica WT (□) and Y. lipolytica mutants overexpressing native (ylHXK1 (▪)—YALI0B22308g) and S. cerevisiae (scHXK2 ( [symbol] )—YGL253W) hexokinases. Yeast were grown in YNB medium with 6% fructose as carbon source with C/N ratio=60. In red: the improvement in FA production (% of CDW; ratio of HXK to WT).

FIG. 8. Sugar utilization by Y. lipolytica W29 (A,B) and its ylHXK1-overexpression transformant (C,D) in YNB fructose medium with different C/N molar ratios (A,C) and rich YP medium with different fructose concentrations (B,D).

FIG. 9. Y. lipolytica phenotype and lipid body development during lipid biosynthesis in stirred-tank bioreactor cultures (150 g·L⁻¹ of sucrose, C/N=60)

FIG. 10. Functional characterization of Y. lipolytica hexose transporter YHT1, YHT2 and YHT3 from the wild type W29 strain. Growth assay of EBY.VW4000 overexpressing the indicated transporters. Cells were pregrown in selective YNB 2% maltose medium. Serial dilutions of washed cells were dropped on solid YNB maltose, glucose and fructose medium as indicated. Cells were grown at 28° C. for 7 days. A) Growth analysis of strains expressing YHT1 and YHT2 genes. Empty vector (1); C06424 (2, 3) and C08943 (4, 5) under the ADH1 (A) or TEF (T) promoter. B) Growth analysis of strains expressing YHT3 from W29, H222 and A101 under the ADH1 (1, 3, 5) and TEF (2, 4, 6) promoter.

FIG. 11. Functional characterization of Y. lipolytica hexose transporter from the wild type H222 and W29 strains. Growth assay of EBY.VW4000 overexpressing the indicated transporters. Cells were pregrown in YNB 2% maltose. Serial dilutions of washed cells were dropped on solid YNB 2% maltose media. Cells were grown at 28° C. for 7 days.

FIG. 12. Functional characterization of Y. lipolytica hexose transporter from the wild type H222 and W29 strains. Growth assay of EBY.VW4000 overexpressing the indicated transporters. Cells were pregrown in YNB 2% maltose. Serial dilutions of washed cells were spotted on solid YNB media with the indicated carbon sources and concentrations. Cells were grown at 28° C. for 7 days.

FIG. 13. Growth curves of EBY.VW4000 overexpressing the indicated transporters from Y. lipolytica WT strains H222. S. cerevisiae YHT overexpression transformants grown in YNB medium with 10 g·L⁻¹ glucose (blue line) or 10 g·L⁻¹ fructose (red line) or 10 g·L⁻¹ glucose-fructose mixture (green line). Growth was analyzed using a Biotek apparatus.

FIG. 14. a) Growth curves and sugar consumption in fructose media supplemented with various glucose concentration. Transformants of EBY.VW4000 overexpressing the indicated transporters from Y. lipolytica WT strain H222 were grown in YNB fructose-glucose media. a) Growth was analyzed in flasks at 28° C. with 10 g·L⁻¹ fructose (F1%, blue line) or in the presence of 1, 5 and 10 g·L⁻¹ glucose (F1% G0.1%, red line; F1% G0.5%, green line; F1% G1%, violet line, respectively); b) Growth curves and sugar consumption in fructose media supplemented with various glucose concentration. Transformants of EBY.VW4000 overexpressing the indicated transporters from Y. lipolytica WT strain H222 were grown in YNB fructose-glucose media. b) Sugar concentration in the media during time. Glucose (Blue line) and fructose (red line).

FIG. 15. Transcription profiles for YHT and D01111 genes during cultivation in minimal medium supplemented with fructose at two concentrations. Transcripts were detected by RT-PCR in the preculture just before inoculation (P) or after inoculation at the time indicated above the wells (h), for strain W29 (panel A) or for strain H222 (panel B).

### EXAMPLES

Characterization of the Y. Lipolytica Hexokinase Gene

### 1. MATERIALS AND METHODS

1.1. Yeast Strains and Plasmids

The plasmids and strains used in this study are listed in Table 4. Three Y. lipolytica wild-type (WT) strains were used (country of origin in parentheses): W29 (France), A-101 (Poland), and H222 (Germany) (Wojtatowicz and Rymowicz, 1991; Barth and Gaillardin, 1996). The following auxotrophic strains had previously been derived from these WT strains and were also used in this study: PO1d (Ura⁻Leu⁻) from W29 (Barth and Gaillardin 1996), A-101-A18 (Ura⁻) from A-101 (Walczak and Robak, 2009), and Y322 (Ura⁻) from H222 (Mauersberger et al., 2001). The other strains used in this study were strains Y3573, Y3812, and Y3850, which contained an expression cassette that included the Y. lipolytica HXK1 gene from W29 (ylHXK1, YALI0B22308g) under the control of the constitutive TEF promoter (Muller et al., 1998), and strain Y3572, which contained an expression cassette carrying the S. cerevisiae hexokinase gene HXK2 (scHXK2, YGL253W). Transformation of Y. lipolytica was performed with the lithium acetate procedure (Xuan et al., 1990), using NotI digested fragments for random chromosomal integration (Mauersberger et al., 2001).

TABLE 4
Strains used in this study. For simplification purposes, the transformants of
three different origin of Y. lipolytica overexpressing hexokinase are named: W29-
HXK1, A-101-HXK1 and H222-HXK1, respectively. Additionally, strains named in the
table e.g. JMY3501are named Y3501.

| | | |
|---|---|---|
| Name | Relevant genotype | Reference |

E. coli strains

| | | |
|---|---|---|
| JME547 | pUB4-CRE 1 | Fickers et al. |
| | | (2003) |
| JME1128 | JMP62 pTEF-GPD1-URA3ex | Dutermo and |
| | | Nicaud (2011) |
| JME1364 | pKS P-LEU2ex-T TGL4 | Dutermo et al. |
| | | (2013) |
| JME1822 | JMP62 pTEF-DGA2-LEU2ex | |
| JME2103 | JMP62 pTEF-YlHXK1-URA3ex | This work |
| JME2347 | JMP62 pTEF-SUC2-LEU2ex | Lazar et al. |
| | | (2013) |
| JME2441 | JMP62 pTEF-ScHXK2-Ura3ex | This work |

Y. lipolytica strains

| | | |
|---|---|---|
| A-101 | MATa WT | Wojtatowicz |
| | | and Rymowicz |
| | | (1991) |
| H222 | MATa WT | Barth and |
| | | Gaillardin |
| | | (1996) |
| W29 | MATa WT | Barth and |
| | | Gaillardin |
| | | (1996) |
| PO1d | MATa ura3-302 leu2-270 xpr2-322 + pXPR2-SUC2 | Barth and |
| | | Gaillardin |
| | | (1996) |
| A-101- | MATa ura3-302 + pXPR2-SUC2 | Walczak and |
| A18 | | Robak (2009) |
| Y322 | MATa ura3-302 + pXPR2-SUC2(H222) | Mauersberger |
| | | et al. (2001) |
| JMY1233 | MATa ura3-302 leu2-270 xpr2-322 Δpox1-6 + pXPR2-SUC2 | Beopoulos et |
| | | al. (2008) |
| JMY2179 | MATa ura3-302 leu2-270 xpr2-322 Δpox1-6 Δtgl4::URA3ex + | This work |
| | pXPR2-SUC2 | |
| JMY2900 | MATa ura3-302 xpr2-322 + URA3ex + pXPR2-SUC2 | Brunel and |
| | | Nicaud |
| | | (unpublished |
| | | data) |
| JMY3122 | MATa ura3-302 leu2-270 xpr2-322 Δpox1-6Δtgl4 + pXPR2-SUC2 | This work |
| JMY3373 | MATa ura3-302 leu2-270 xpr2-322 Δpox1-6Δtgl4 + pXPR2- | This work |
| | SUC2 + pTEF-DGA2-LEU2ex | |
| JMY3501 | MATa ura3-302 leu2-270 xpr2-322 Δpox1-6 Δtgl4 + pXPR2- | This work |
| | SUC2 + pTEF-DGA2-LEU2ex + pTEF-GPD1-URA3ex | |
| JMY3572 | MATa ura3-302 leu2-270 xpr2-322 + pXPR2-SUC2 + pTEF-ScHXK2- | This work |
| | URA3ex + LEU2ex | |
| JMY3573 | MATa ura3-302 leu2-270 xpr2-322 + pXPR2-SUC2 + pTEF-YlHXK1- | This work |
| | URA3ex + LEU2ex | |
| JMY3812 | MATa ura3-302 + pXPR2-SUC2 + pTEF-YlHXK1-URA3ex (A-101) | This work |
| JMY3820 | MATa ura3-302 leu2-270 xpr2-322 Δpox1-6 Δtgl4 + pXPR2- | This work |
| | SUC2 + pTEF-DGA2 + pTEF-GPD1 | |
| JMY3850 | MATa ura3-302 + pXPR2-SUC2 + pTEF-YlHXK1-URA3ex (H222) | This work |
| JMY4059 | MATa ura3-302 leu2-270 xpr2-322 Δpox1-6 Δtgl4 + pXPR2- | This work |
| | SUC2 + pTEF-DGA2 + pTEF-GPD1 + pTEF-YlHXK1-URA3ex | |
| JMY4086 | MATa ura3-302 leu2-270 xpr2-322 Δpox1-6 Δtgl4 + pXPR2- | This work |
| | SUC2 + pTEF-DGA2 + pTEF-GPD1 + pTEF-YlHXK1-URA3ex + pTEF-SUC2- | |
| | LEU2ex | |

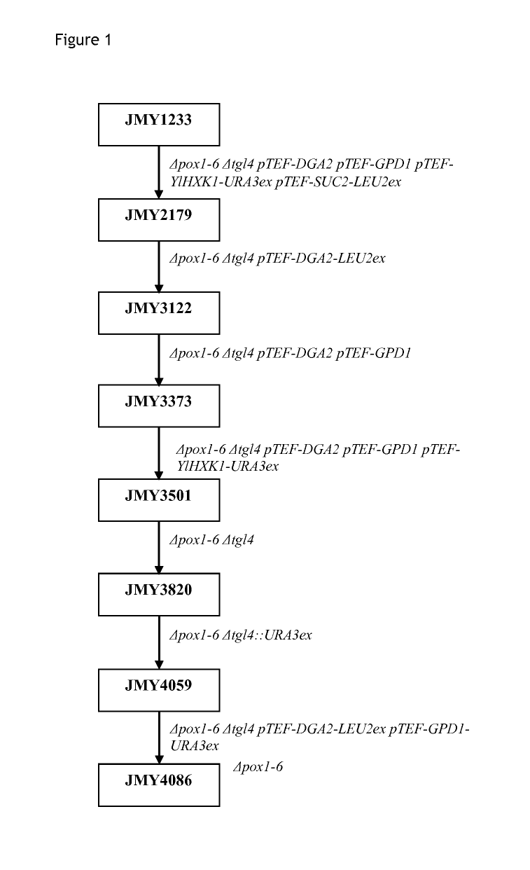

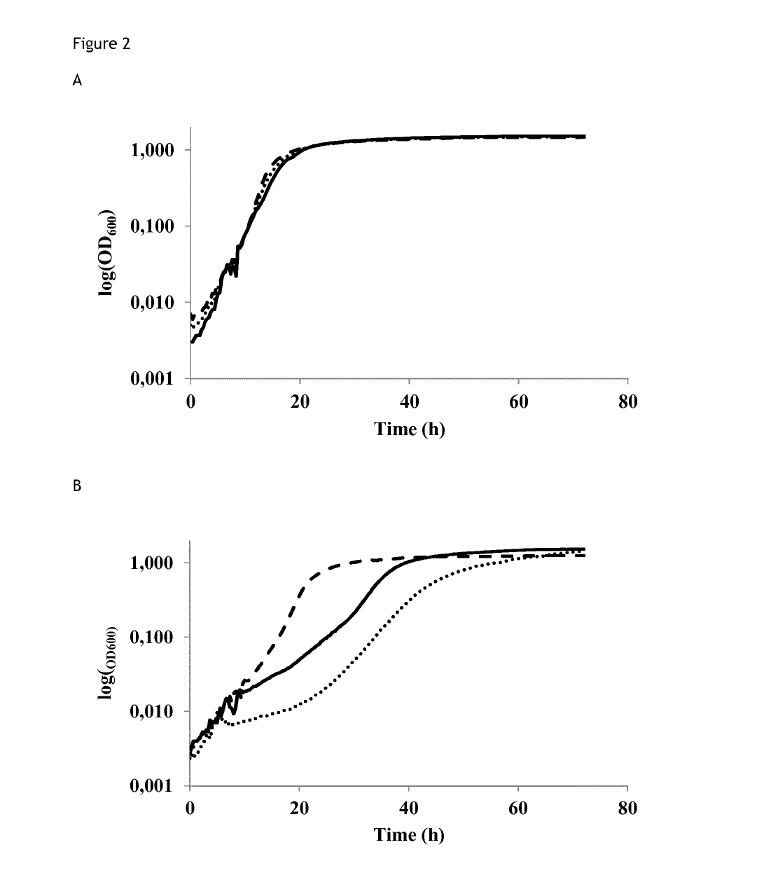

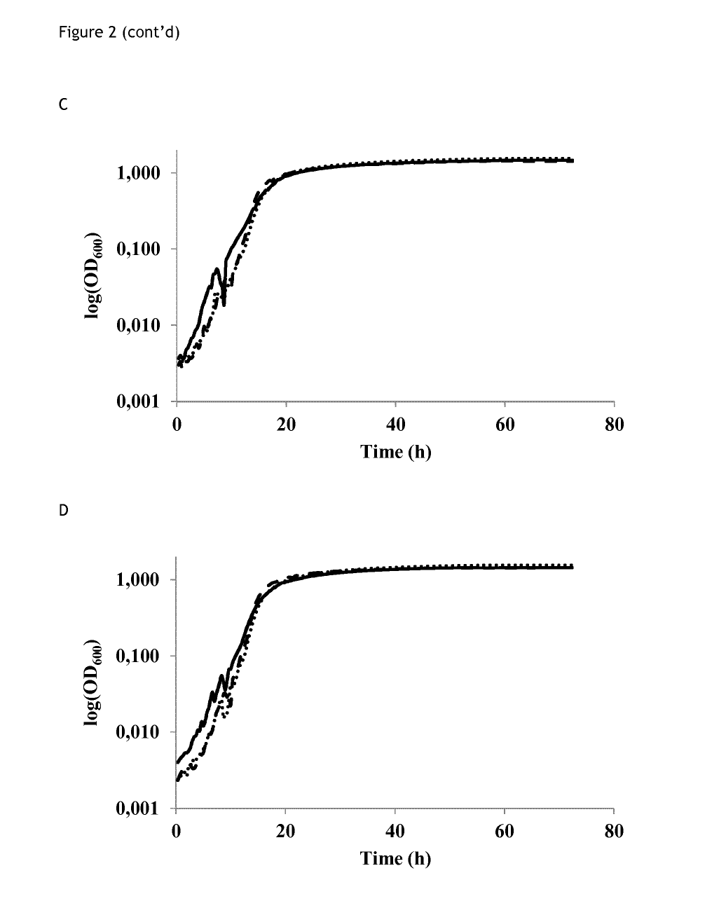

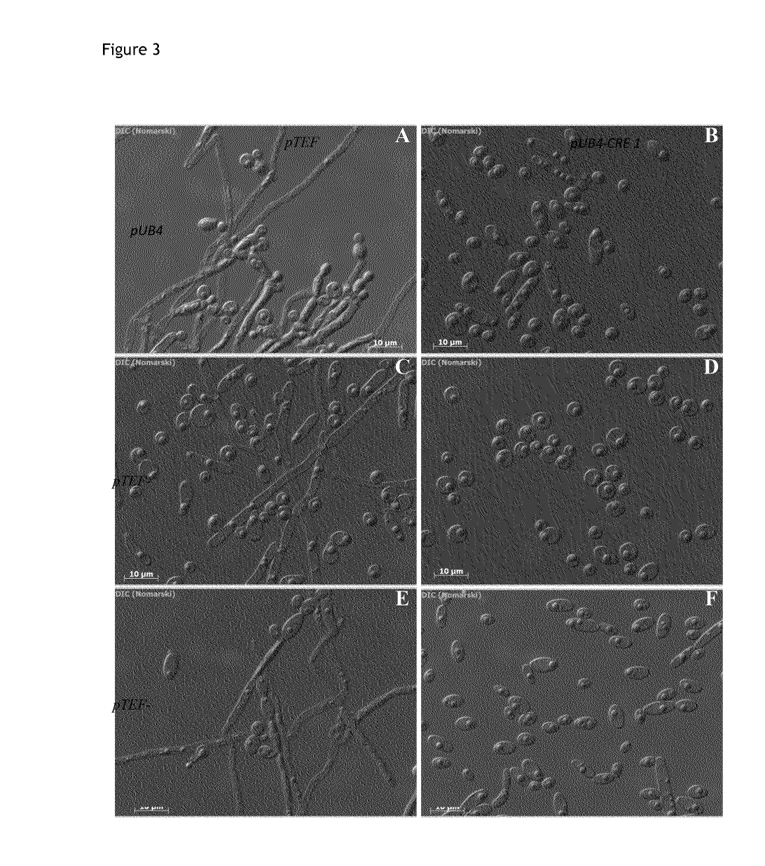

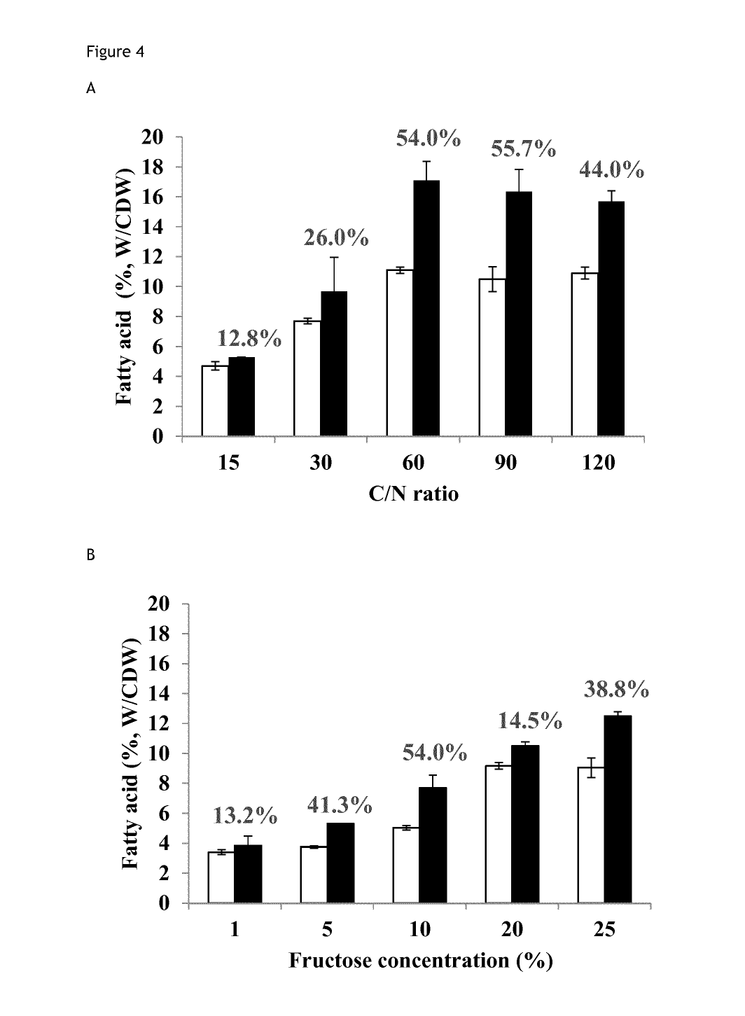

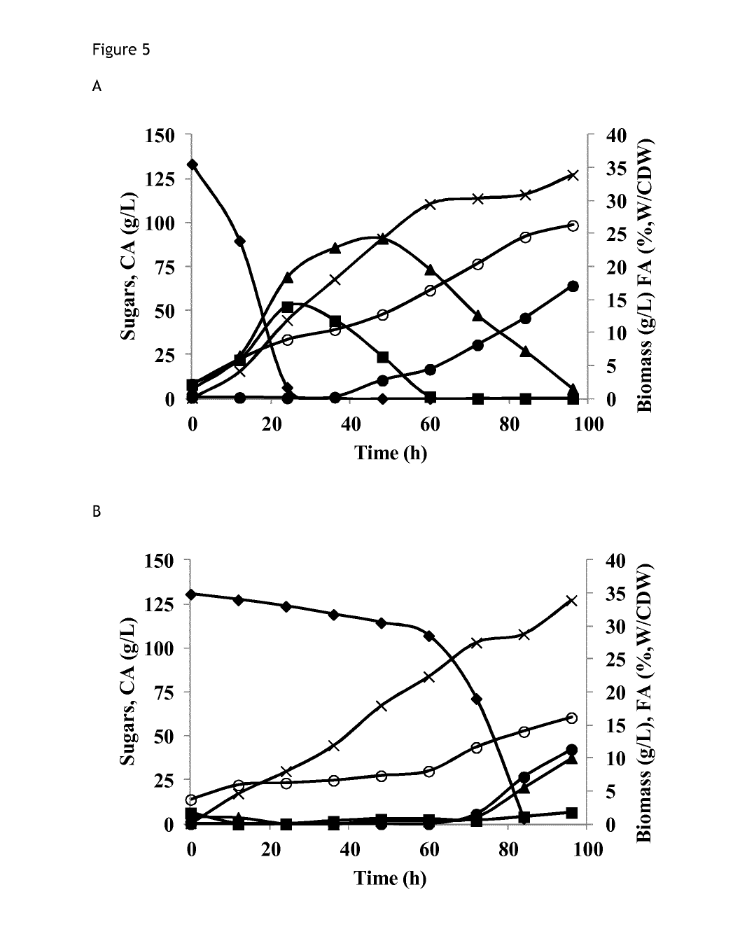

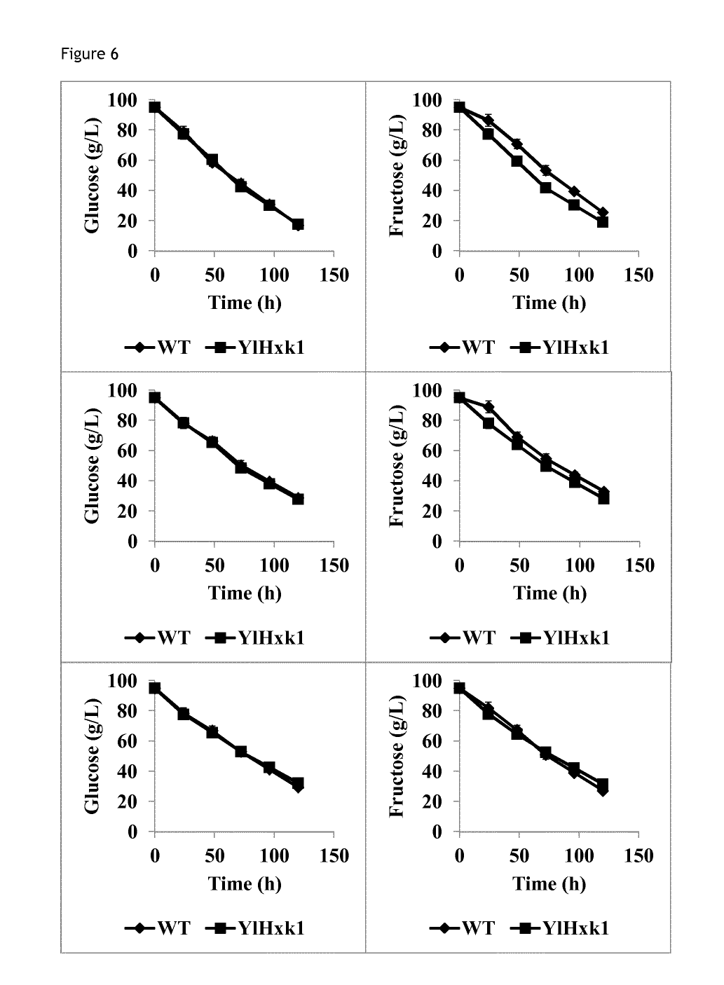

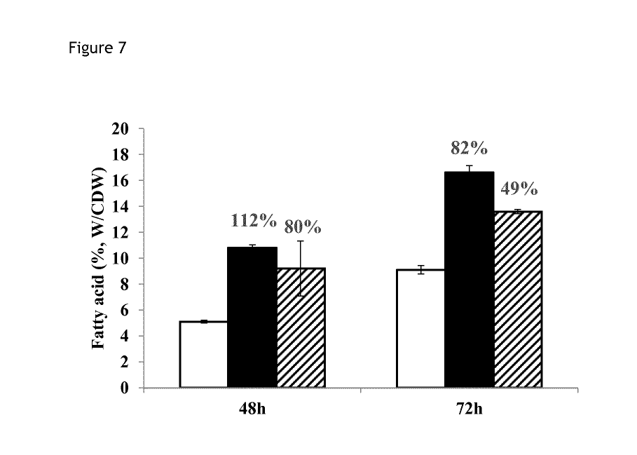

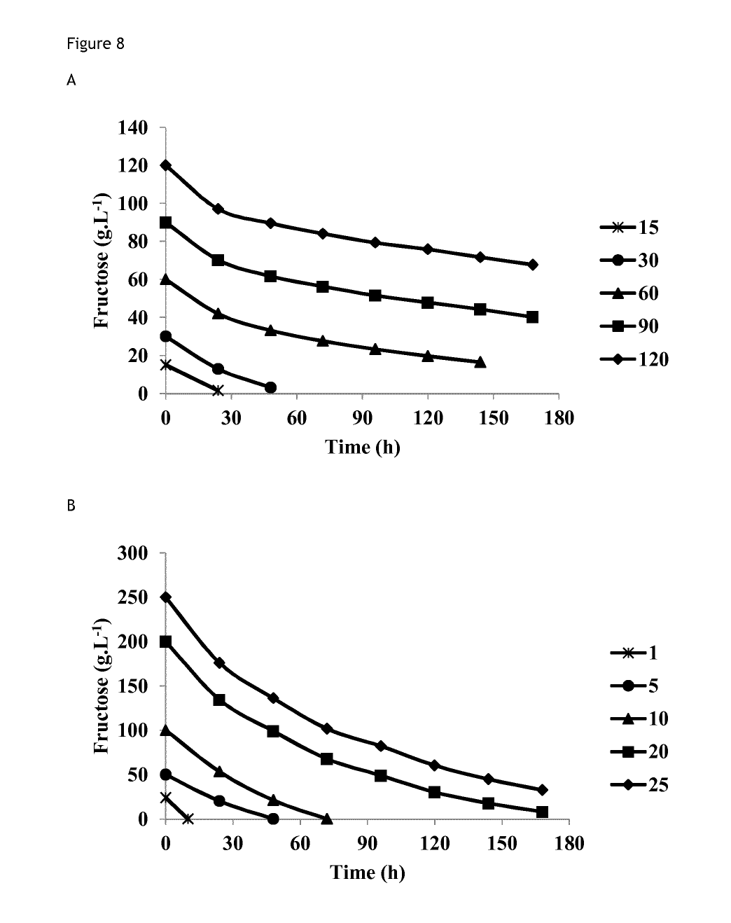

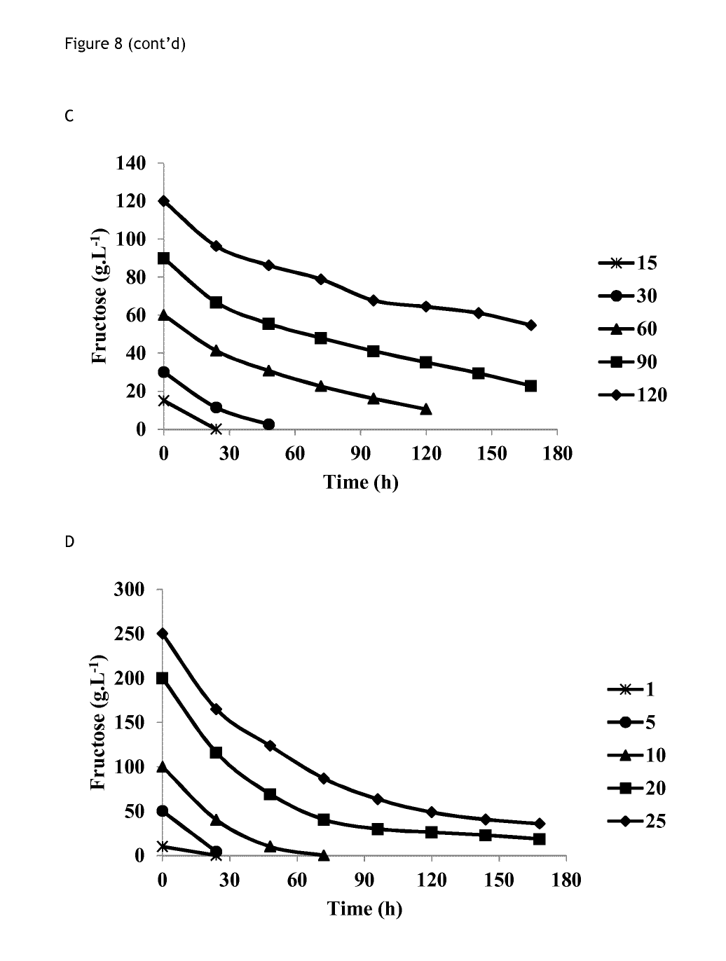

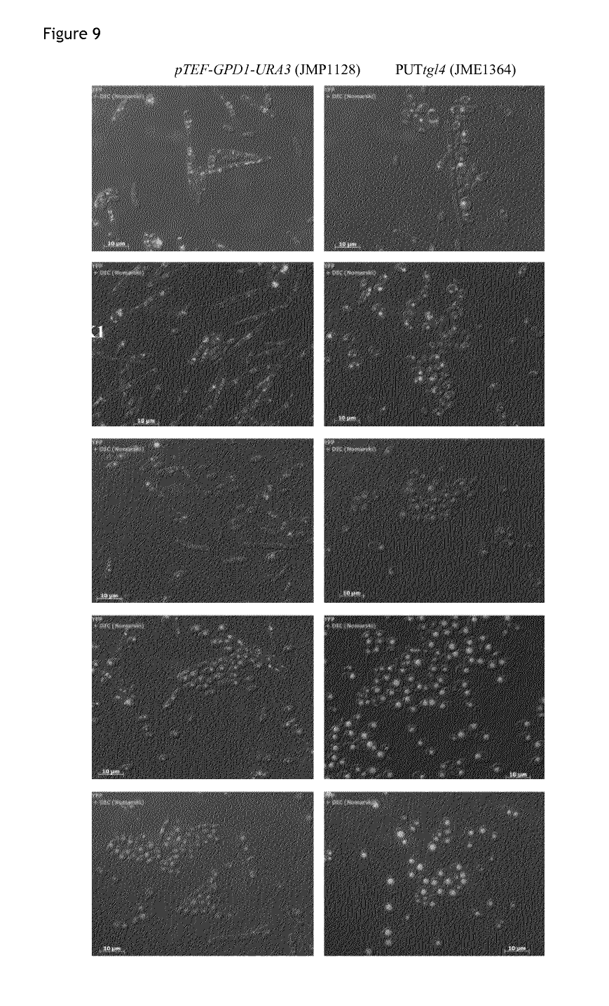

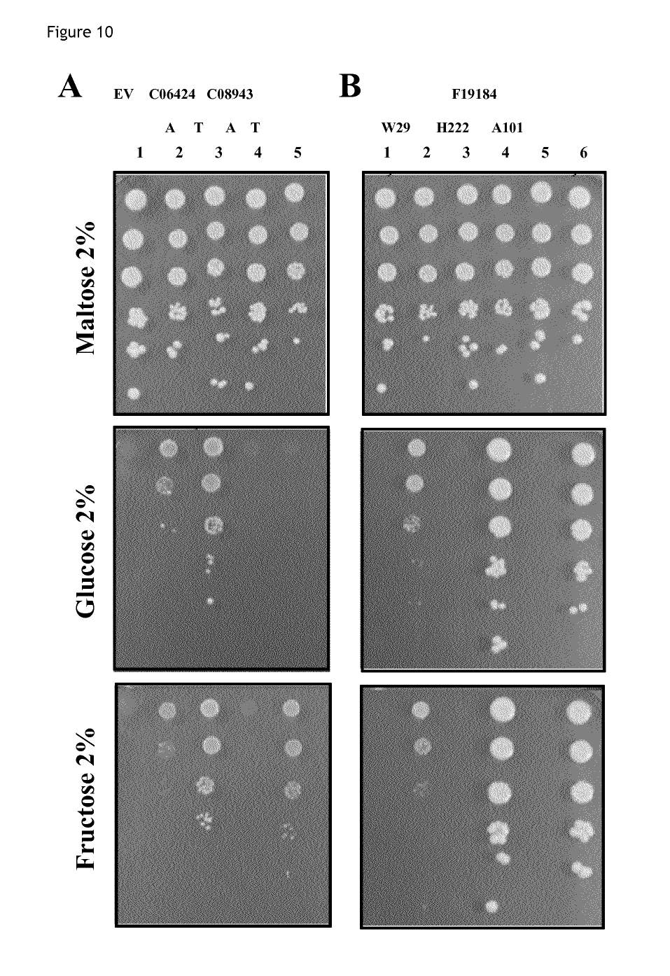

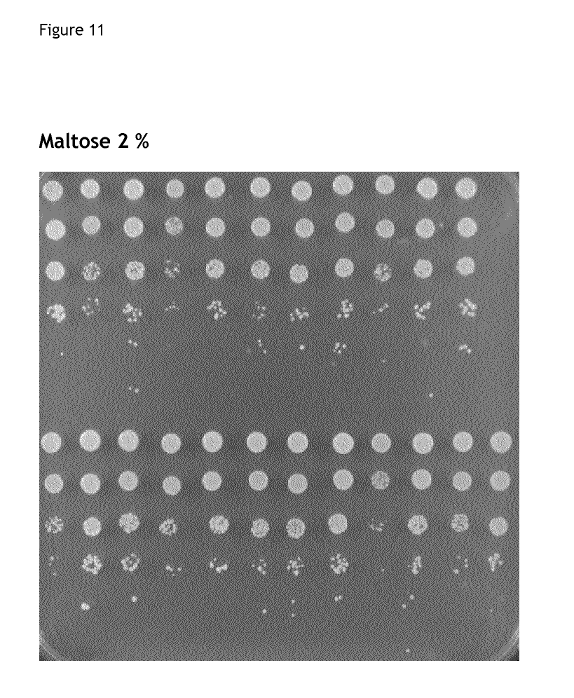

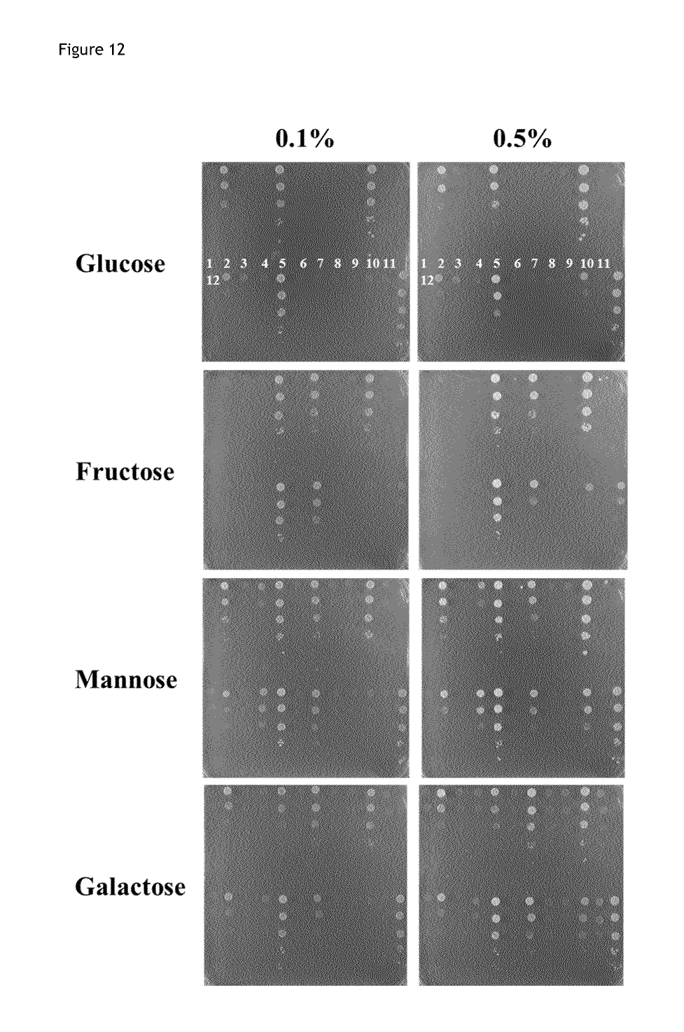

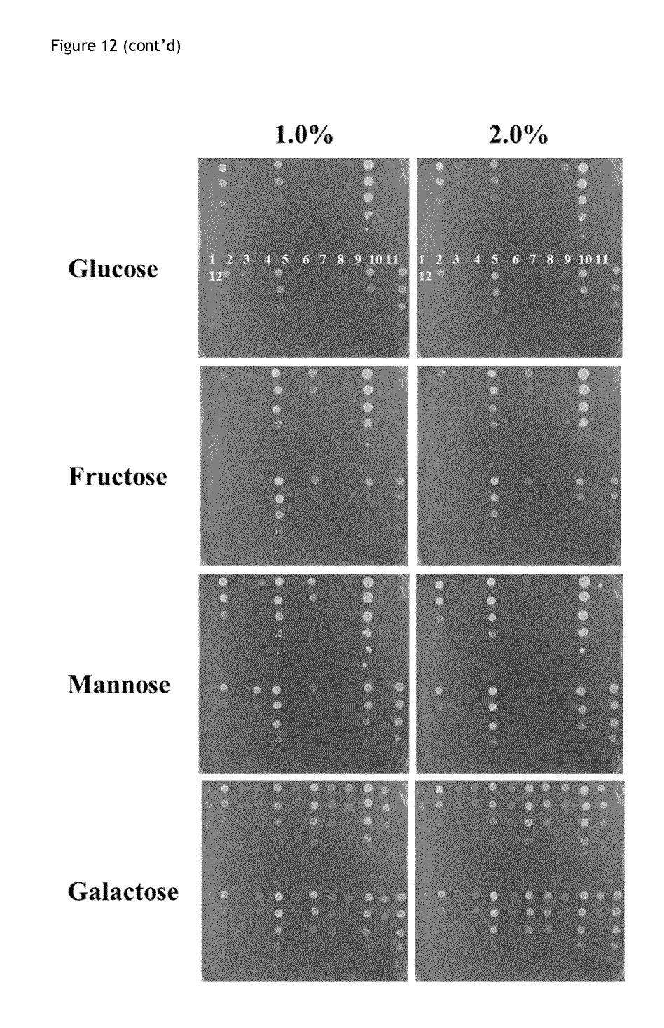

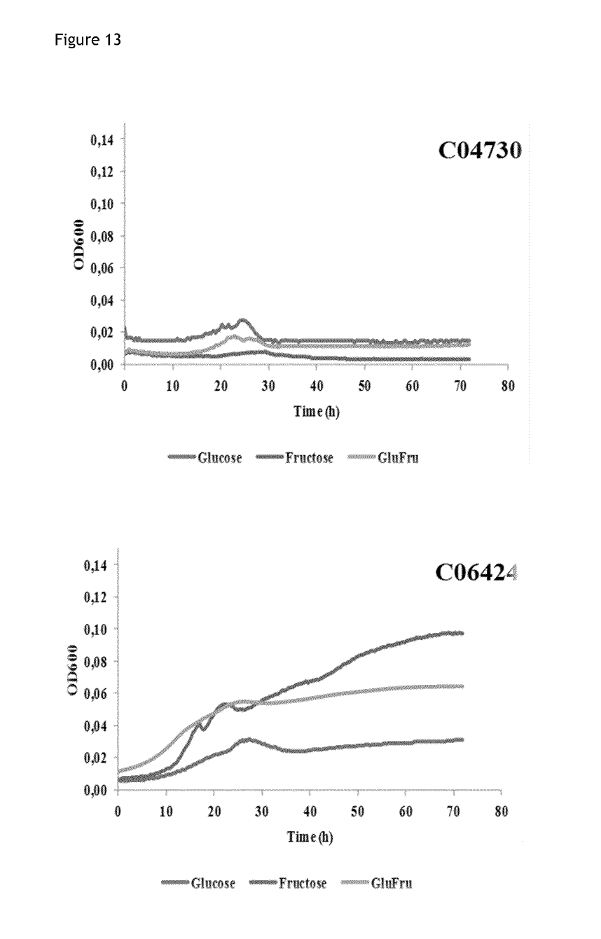

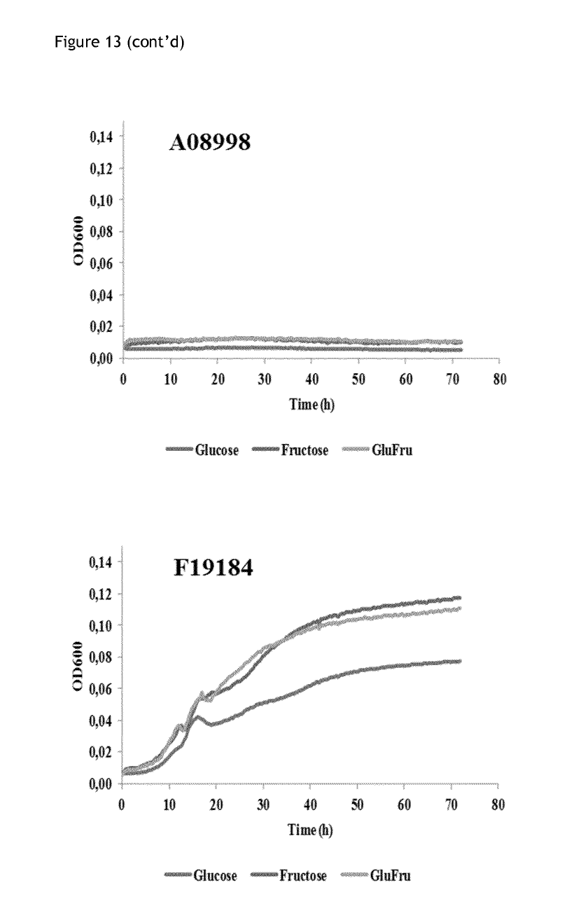

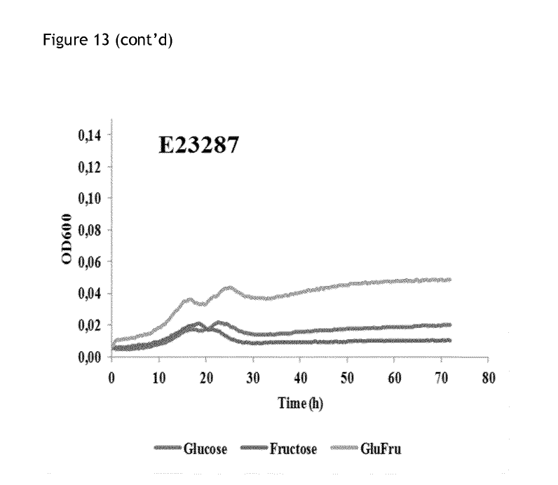

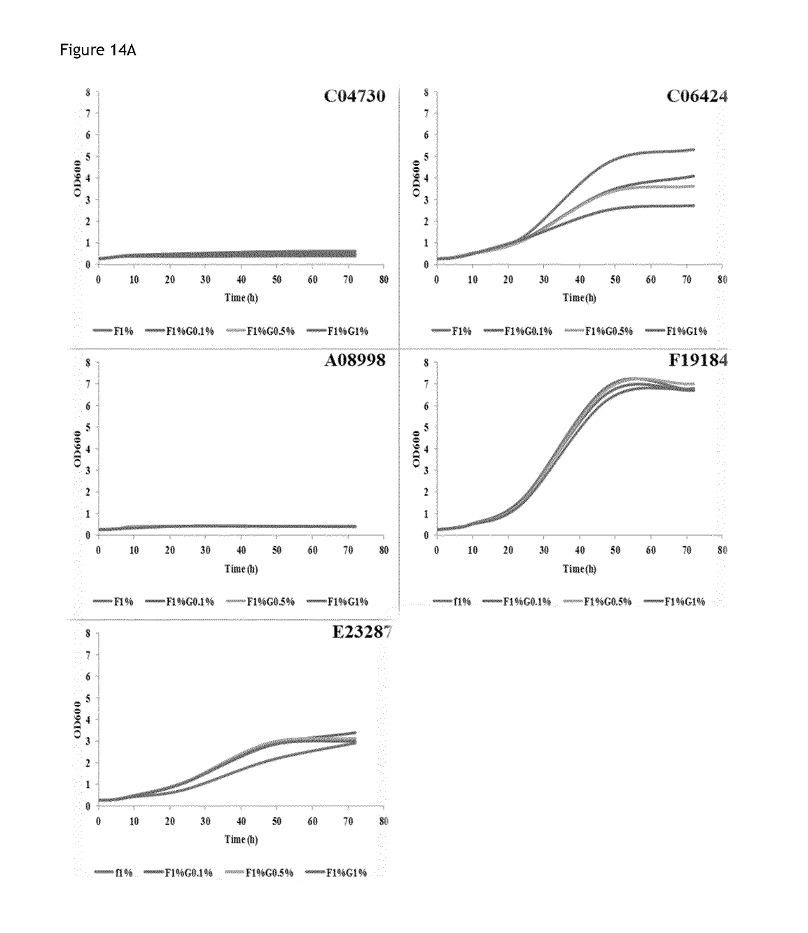

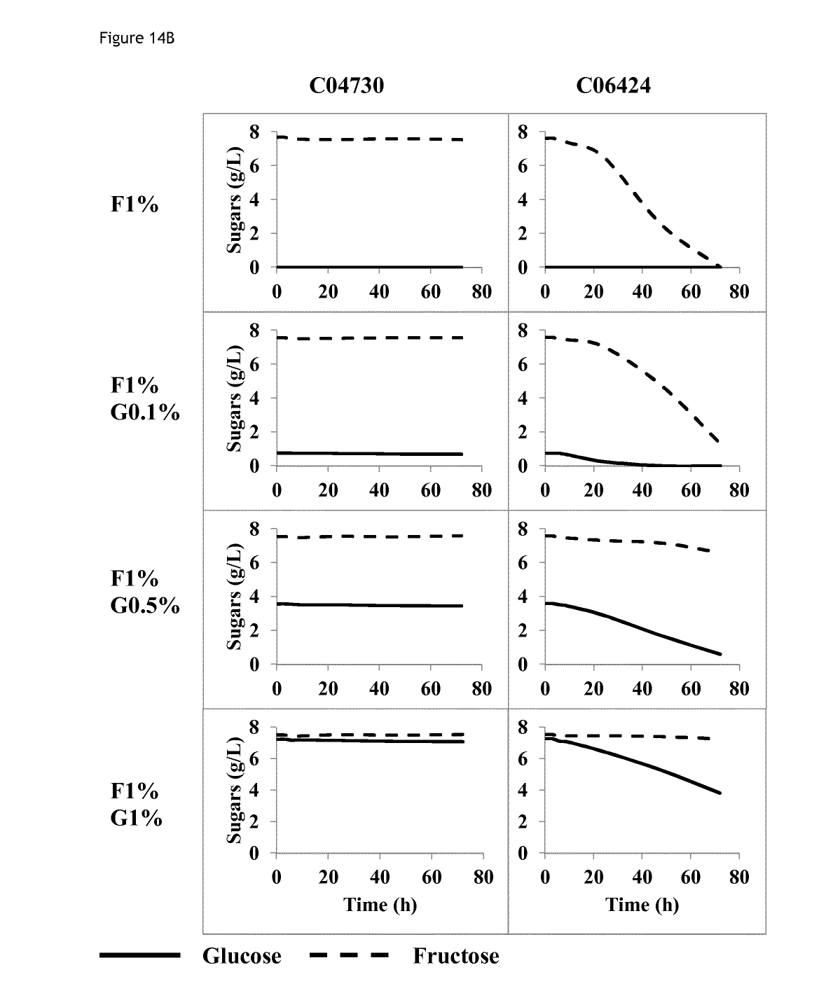

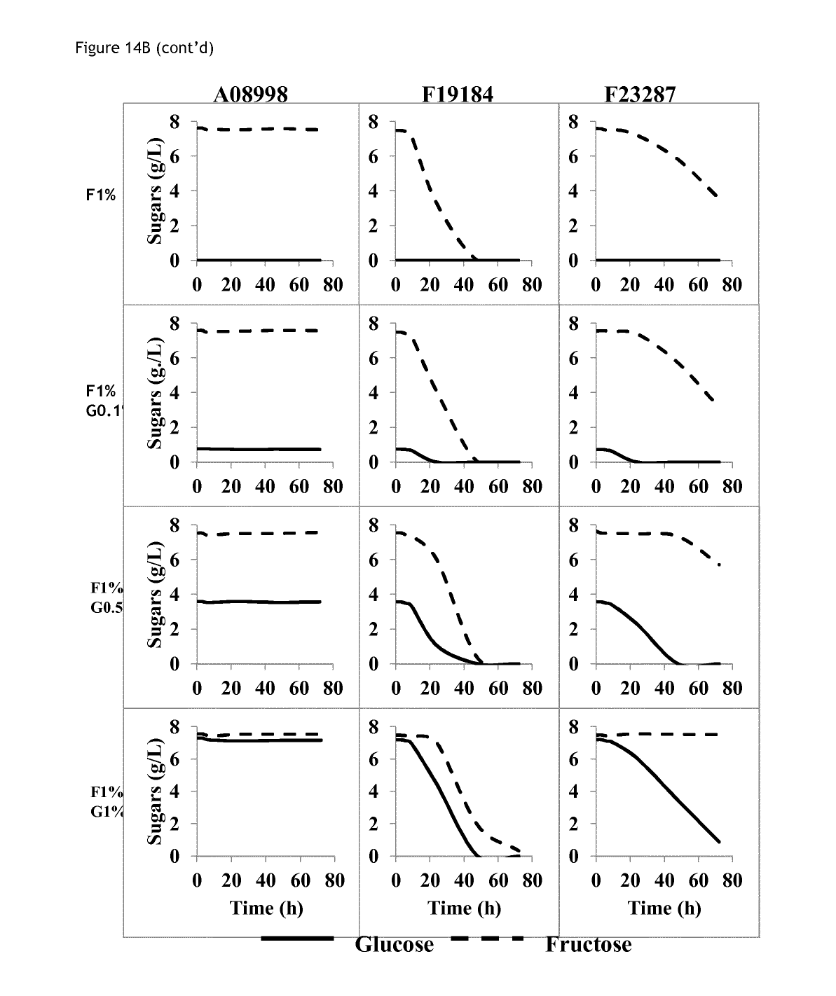

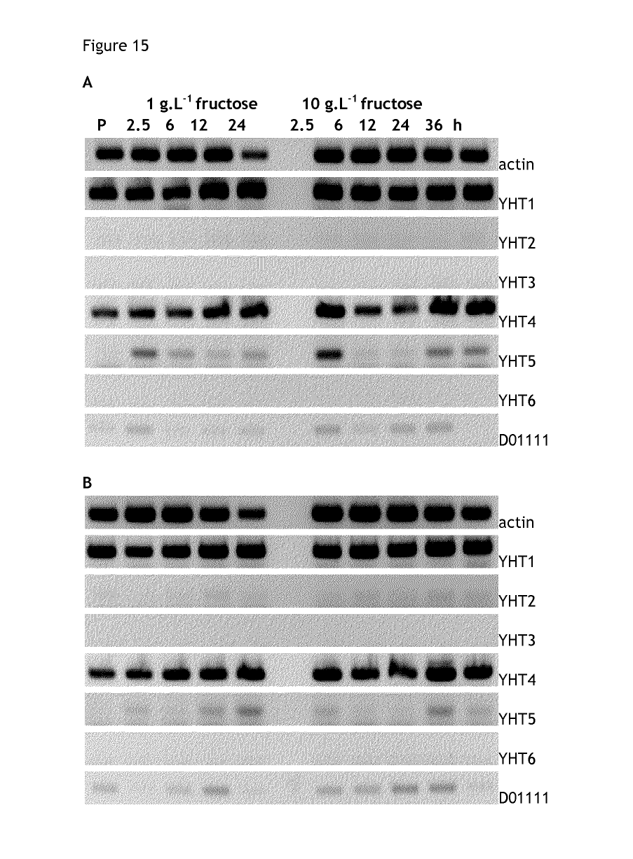

To recover prototrophy, strains Y3572 and Y3573 were transformed with a purified Sail fragment of the plasmid pINA62 that contained the LEU2 gene (Gaillardin and Ribet, 1987). Construction of the Y4086 strain, which was modified for lipid production, is depicted in detail in FIG. 1.

1.2. Growth Media

Media and growth conditions for Escherichia coli were identical to those in previous studies (Sambrook and Russell, 2001), as were those of Y. lipolytica (Barth and Gaillardin, 1996). Rich (YPD) medium was prepared using 20 g·L⁻¹ Bacto™ Peptone (Difco, Paris, France), 10 g·L⁻¹ yeast extract (Difco), and 20 g·L⁻¹ glucose (Merck, Fontenay-sous-Bois, France). Minimal (YNB) medium was prepared using 1.7 g·L⁻¹ yeast nitrogen base (without amino acids and ammonium sulphate, Difco), 10 g·L⁻¹ glucose (Merck), 5 g·L⁻¹ NH₄Cl, and 50 mM phosphate buffer (pH 6.8). To complement auxotrophic processes, 0.1 g·L⁻¹ uracil or leucine (Difco, Paris, France) were added as necessary.

1.3. Growth in Microtiter Plates

Precultures were obtained from frozen stocks, inoculated into tubes containing 5 mL YPD medium, and cultured overnight (170 rpm, 28° C.). Precultures were then centrifuged and washed with sterile distilled water; cell suspensions were standardized to an OD₆₀₀ of 0.1. Yeast strains were grown in 96-well plates in 200 μl of minimal YNB medium (see above) containing 10 g·L⁻¹ of either glucose or fructose.

The culture was performed three times, with 2-3 replicates for each condition. Cultures were maintained at 28° C. under constant agitation with a Biotek Synergy MX microtiter plate reader (Biotek Instruments, Colmar, France); each culture's optical density at 600 nm was measured every 20 min for 72 h.

1.4. Media and Growth for Lipid Biosynthesis Experiments

For lipid biosynthesis in minimal media, cultures were prepared as follows: an initial preculture was established by inoculating 50 mL of YPD medium in 250-mL Erlenmeyer flasks; this was followed by an overnight shaking step at 28° C. and 170 rpm. The resulting cell suspension was washed three times with sterile distilled water and used to inoculate 50 mL of YNB medium containing 15, 30, 60, 90, or 120 g·L⁻¹ of fructose (corresponding to a carbon/nitrogen (C/N) ratio of 15, 30, 60, 90, and 120, respectively). Each culture was incubated, with shaking, in non-baffled 250-mL Erlenmeyer flasks, at 28° C. and 170 rpm for 168 h, or until all available sugar had been consumed. We also evaluated lipid biosynthesis in several other types of media, including a glucose-only (60 g·L⁻¹, C/N=60) control medium, a sucrose-containing (60 g·L⁻¹, C/N=60) medium, and a rich fructose (YPF) medium. The latter was prepared using 20 g·L⁻¹ peptone; 10 g·L⁻¹ yeast extract; and 10, 50, 100, 200, or 250 g·L⁻¹ of fructose. The preculture and growth conditions for each experiment were as described above.

1.5. Bioreactor Studies

Lipid biosynthesis was also evaluated in batch cultures (BC) that were maintained for 96 h in 5-L stirred-tank BIO-STAT B-PLUS reactors (Sartorius, Frankfurt, Germany) under the following conditions: 2-L working volume, 28° C., 800 rpm, and 4-L·min⁻¹ aeration rate. The production media contained 150 g sucrose, 1.7 g YNB, 3.75 g NH₄Cl, 0.7 g KH₂PO₄, and 1.0 g MgSO₄×7H₂O, all in 1 L of tap water. Culture acidity was automatically maintained at pH 6.8 using a 40% (w/v) NaOH solution. Culture inocula were grown in 0.1 L of YNB medium with 30 g·L⁻¹ glucose in 0.5-L flasks on a rotary shaker kept at 170 rpm and 28° C. for 48 h; inocula were added to the bioreactor cultures in a volume equal to 10% of the total working volume.

To analyze lipid production in the bioreactor cultures, a 15-mL sample was taken from each culture 10 min after inoculation (Time=0); subsequent sampling was conducted every 12 hours. Each sample was centrifuged for 10 min at 5000 rpm; supernatants and cell pellets were collected and used for further analyses.

1.6. General Genetic Techniques and Plasmid Construction

Standard molecular genetics techniques were used throughout this study following Sambrook et al. (1989). Restriction enzymes were obtained from New England Biolabs (Ipswich, England). Genomic DNA from yeast transformants was prepared as described by Querol et al. (1992). PCR amplification was performed using an Eppendorf 2720 thermal cycler and employing both Taq (Promega, Madison, Wis.) and Pfu (Stratagene, La Jolla, Calif.) DNA polymerases. PCR fragments were then purified with a QIAgen Purification Kit (Qiagen, Hilden, Germany), and DNA fragments were recovered from agarose gels using a QIAquick Gel Extraction Kit (Qiagen, Hilden, Germany). The Staden software package was used for gene sequence analysis (Dear and Staden, 1991).To quantify hexokinase gene expression, genes were amplified with the primer pairs ylHXK1-fwd and ylHXK1-rev (GAGAAGATCTATGGTTCATCTTGGTCCCCGAAAACCC, SEQ ID NO: 38 and GCGCCCTAGGCTAAATATCGTACTTGACACCGGGCTTG, SEQ ID NO: 39, respectively), and scHXK2-fwd and scHXK2-rev (SEQ ID NO: 4 0: GCGCGGATCCATGGTTCATTTAGGTCCAAAAAAACC and SEQ ID NO: 41: GCGCCCTAGGTTAAGCACCGATGATACCAACG, respectively), all of which contained BamHI(BglII)-AvrII restriction sites. These restriction sites enabled the genes to be cloned into JME1128 plasmids that had been digested with BamHI-AvrII, as previously described (Beopoulos et al. 2008; Dulermo et al., 2011). To delete the genes of interest, the disruption cassettes were produced in accordance with the protocol of Fickers and colleagues (2003). Auxotrophies were restored via excision using the Cre-lox recombinase system following transformation with the replicative plasmid pUB4-Cre1 (JME547) (Fickers et al., 2003).

1.7. Fluorescence Microscopy

Images were obtained using a Zeiss Axio Imager M2 microscope (Zeiss, Le Pecq, France) with a 100× objective lens and Zeiss filter sets 45 and 46 for fluorescence microscopy. Axiovision 4.8 software (Zeiss, Le Pecq, France) was used for image acquisition. To make the lipid bodies (LBs) visible, BodiPy® Lipid Probe (2.5 mg·mL⁻¹ in ethanol; Invitrogen) was added to the cell suspension (OD₆₀₀=5) and the samples were incubated for 10 min at room temperature.

1.8. Lipid Determination

Fatty acids (FAs) in 15-mg aliquots of freeze-dried cells were converted into methyl esters using the method described in Browse et al. (1986) and were analyzed using a gas chromatograph (GC). GC analysis of FA methyl esters was performed using a Varian 3900 instrument equipped with a flame ionization detector and a Varian FactorFour vf-23ms column, for which the bleed specification at 260° C. was 3 pA (30 m, 0.25 mm, 0.25 μm). FAs were identified by comparing their GC patterns to those of commercial FA methyl ester standards (FAME32; Supelco) and quantified using the internal standard method, which involved the addition of 50 μg of commercial C17:0 (Sigma).

Total lipid extractions were obtained from 100-mg samples (cell dry weight (CDW)) in accordance with the method described by Folch et al. (1957). Briefly, Y. lipolytica cells were spun down, washed with water, freeze dried, and then resuspended in a 2:1 chloroform/methanol solution and vortexed with glass beads for 20 min. The organic solution was extracted and washed with 0.4 mL of 0.9% NaCl solution before being dried at 60° C. overnight and weighed to quantify lipid production.

1.9. Sugar and Citric Acid Measurement

Citric acid (CA), glucose, fructose, and sucrose were identified and quantified by HPLC (UltiMate 3000, Dionex-Thermo Fisher Scientific, UK) using an Aminex HPX87H column coupled to UV (210 nm) and RI detectors. The column was eluted with 0.01 N H₂SO₄ at room temperature and a flow rate of 0.6 mL·min⁻¹. Identification and quantification were achieved via comparisons to standards. Before being subject to HPLC analysis, samples were filtered on 0.45-μm pore-size membranes.

1.10. Dry Biomass Determination

To determine dry biomass, the cell pellets from 15-mL culture samples were washed twice with distilled water, filtered on 0.45-μm pore-size membranes, and dried at 105° C. using a WPS 1105 weight dryer (Radwag, Poznań, Poland) until a constant mass was reached.

1.11. Measurement of Hexokinase Activity

Total hexokinase activity was determined using whole cell extracts and a Hexokinase Colorimetric Assay Kit (Sigma-Aldrich, Saint Louis, Mo., USA) in accordance with the manufacturer's instructions. The reaction was performed at 24° C. in 96-well plates using a Biotek Synergy MX microtiter plate reader and was monitored by measuring absorbance at 450 nm. One unit of hexokinase was defined as the amount of enzyme that generated 1.0 μmole of NADH per minute at pH 8.0 at room temperature.

1.12. Reverse Transcription and qRT-PCR

RNA extraction was performed using TRIzol® reagent (Invitrogen, Carlsbad, Calif., USA) in accordance with the manufacturer's instructions. Nucleic acids amounts were measured using a Biochrom WPA Biowave II spectrophotometer (Biochrom Ltd., Cambridge, UK) equipped with a TrayCell (HelmaAnalytics, Müllheim, Germany). Following the manufacturer's instructions, cDNA was prepared using Maxima First Strand cDNA Synthesis Kits for RT-qPCR (ThermoScientific, Waltham, Mass., USA).

Real-time PCR was performed using the DyNAmo Flash SYBR Green qPCR Kit (ThermoScientific, Waltham, Mass., USA) with 0.5 μM forward and reverse primers and 1 μg of cDNA template in a final reaction volume of 10 μL. Thermocycling was performed in the Eco Real-Time PCR System (Illumina, San Diego, Calif., USA) with the following cycling parameters: 5 min incubation at 95° C., followed by 40 cycles of 10 s at 95° C., 10 s at 60° C., and 8 s at 72° C. Fluorescence data were acquired during each elongation step, and at the end of each run, specificity was controlled by melting curve analysis. Hexokinase expression was detected using the primers ylHXK1-qPCR-fwd (SEQ ID NO: 42: TCTCCCAGCTTGAAACCATC) and ylHXK1-qPCR-rev (SEQ ID NO: 43: CTTGACAACTCGCAGGTTGG). The results were normalized to actin gene expression (Lazaret al., 2011) and then analyzed using the ddCT method (Schmittgen and Livak, 2008).

All experiments in this paper were performed at least three times.

### 2. RESULTS AND DISCUSSION

Y. lipolytica is a strictly aerobic microorganism that is known to grow on hydrophobic substrates like n-alkanes, fatty acids, and oils (Fickers et al., 2005b). This yeast has been reported to metabolize a few types of different sugars, namely glucose, fructose, and mannose (Coelho et al., 2010; Michely et al., 2013), and its preferential consumption of glucose over fructose has been well-described (Wojtatowicz et al., 1997; Lazar et al., 2011).

2.1. Variability of Fructose Utilization Among Y. Lipolytica Strains of Different Origin

The ability of three wild-type strains of different origins, i.e., W29 (France), H222 (Germany), and A-101 (Poland), to grow in media containing either glucose or fructose was compared. The three wild-type strains presented similar growth kinetics in YNB containing 10 g·L⁻¹ glucose (μ=0.355 h⁻¹; FIG. 2A), but their growth kinetics differed significantly in the medium containing 10 g·L⁻¹ fructose (FIG. 2B). In the fructose medium, H222 displayed a constant growth rate (0.282 h⁻¹), while both A-101 and W29 clearly showed reduced growth rates and distinct phases of growth. In the first few hours of culture, cells from all three strains grew at approximately equal rates; however, beginning at approximately 8 h of culture, both A101 and W29 exhibited biphasic growth profiles. In A-101, the first phase was characterized by a slow growth rate from 8 to 20 h (0.131 h⁻¹), while growth during the second phase was faster, around 0.203 h⁻¹ (although this rate was not constant and could even be interpreted as occurring in two distinct sub-phases). In W29, there was a clear period of very slow growth, from 8 to 20 h, followed by a phase of exponential growth (0.182 h⁻¹). Altogether, it is clear that the three wild-type strains each responded quite differently to the fructose medium by exhibiting different rates and phases of growth.

2.2. Overexpression of the ylHXK1 Gene Enhances Hexokinase Activity, Growth, and Fructose Uptake in Y. Lipolytica

As Hxk1p is crucial for fructose assimilation in Y. lipolytica, we reasoned that interstrain variation in its activity could potentially be responsible for the diversity of growth patterns that we observed among the strains grown on fructose. We thus obtained the genome sequences of the different Y. lipolytica strains (Neuvéglise, unpublished data) and compared the hexokinase gene and its promoter region among the three strains (data not shown). No polymorphisms were found in the putative hexokinase sequence, and only a few changes were identified in the different strains' promoter regions.

In an attempt to increase hexokinase activity among the different strains, we decided to introduce an additional copy of ylHXK1 under the strong, constitutive TEF promoter (Muller et al., 1998) into each strain. First, we examined the impact of this addition on the overall abundance of HXK1 transcripts and kinase activity by comparing these ylHXK1 transformants to their WT parental strains after they had been grown in glucose versus fructose media (Table 5). The presence of an additional copy of ylHXK1 increased both HXK1 transcript abundance (at least 23 fold) and hexokinase activity (at least 6 fold) in both carbon sources, which confirmed that constitutive overexpression had been successful. Transformants with the H222 background differed the least from their WT parent, both in terms of their HXK1 transcription levels and their kinase activity, while the largest difference was seen in the W29 transformants—of the three original strains, W29 showed the slowest growth on fructose. After they had been grown in fructose-containing medium, all three ylHXK1-overexpressing strains exhibited similar levels of hexokinase activity, around 1700 U·g_{CDW}⁻¹, an observation that suggests that this value could be the maximum level of hexokinase activity.

TABLE 5
Activity and mRNA fold change of hexokinase extracted from Y. lipolytica
WT and ylHXK1 mutants growing in YNB medium with 100 g · L⁻¹ glucose or 100 g · L⁻¹
fructose analyzed at 24 h of the culture.

| | Glucose | | | Fructose | | |
|---|---|---|---|---|---|---|
| | | | Fold | | | Fold |
| | | | change in | | | change in |
| | Activity | Fold | transcript | Activity | Fold | transcript |
| Strain | (U · g_{CDW}⁻¹) | change | level | (U · g_{CDW}⁻¹) | change | level |
| W29 | 193.5 ± 23 | 7.70 | 28.22 ± 1.7 | 145.3 ± 12 | 12.15 | 96.11 ± 4.8 |
| W29-HXK1 | 1490.4 ± 186 | | | 1766.2 ± 118 | | |
| A-101 | 42.4 ± 3 | 27.10 | 40.21 ± 3.0 | 155.6 ± 10 | 10.73 | 55.39 ± 2.8 |
| A-101- | 1148.6 ± 87 | | | 1670.2 ± 151 | | |
| HXK1 | | | | | | |
| H222 | 33.1 ± 4 | 33.92 | 55.84 ± 3.6 | 256.5 ± 21 | 6.45 | 23.61 ± 1.2 |
| H222-HXK1 | 1122.6 ± 100 | | | 1653.6 ± 181 | | |

Next, we examined the growth capacity of the different transformants as compared to the WT strains. Although we observed a clear increase in hexokinase activity in both glucose- and fructose-based media, the growth profiles of the ylHXK1-overexpressing strains in YNB glucose were similar to those of the WT strains (μ=0.367 h⁻¹; FIG. 2C). In contrast, overexpression of native Y. lipolytica hexokinase significantly improved the growth rate of all the transformants when they were grown in fructose-based medium (μ=0.363 h⁻¹; FIG. 2D). In YNB fructose, all three ylHXK1-overexpressing strains exhibited the same growth kinetics, which were equivalent to those observed when the strains were grown in YNB glucose. This finding means that the ability of the slow-growing strains W29 and A-101 to grow on fructose was immensely improved and suggests that hexokinase activity may be a limiting factor that restricts growth in these WT strains. Interestingly, overexpression of hexokinase II in S. cerevisiae did not stimulate its growth on fructose (Ernandes et al., 1998), suggesting that there are fundamental differences between S. cerevisiae and Y. lipolytica in the regulation of fructose metabolism.

Finally, to investigate the indirect effects of hexokinase overexpression on glucose and fructose assimilation, we analyzed the uptake of these sugars by following changes in their concentration in the medium during yeast culture; their initial concentration was 100 g·L⁻¹ (FIG. 6). Both the original Y. lipolytica WT strains and their ylHXK1 transformants consumed glucose at the same rate (0.65, 0.56, and 0.54 g·L⁻¹·h⁻¹ for W29, A-101, and H222, respectively; FIG. 6: A,C,E). However, at the beginning of the culturing period, fructose was consumed faster by the ylHXK1-overexpressing strains (at a rate of 0.64, 0.56, and 0.55 g·L⁻¹·h⁻¹ for W29, A-101, and H222, respectively) than by the WT strains (0.36, 0.38, and 0.54 g·L⁻¹·h⁻¹ for W29, A-101, and H222, respectively; FIG. 6: B,D,F). After 24 h, fructose consumption rates slowed and became similar for the WT strains and the ylHXK1 transformants, which suggests that hexokinase overexpression achieves its maximal impact at the beginning of growth. As in our analysis of transcript abundance, we observed the largest rate increase in the W29 transformant, whose WT parent grew and consumed fructose slower than the other original strains. It is worth noting that, in the ylHXK1 transformants, fructose consumption rates reached the same levels as glucose consumption rates.

2.3. Overexpression of Hexokinase Inhibits Filamentation of Y. Lipolytica

The cultivation of Y. lipolytica in YNB glucose favored the growth of cells in the yeast form; however, when fructose was the carbon source, filamentation was clearly observed in the three WT strains (FIG. 3: A,C,E), even though conditions were otherwise the same. This phenomenon was more apparent when fructose concentrations were equal to or lower than 10%, and higher concentrations of this sugar seemed to partially inhibit hyphae formation (data not shown). Overexpression of hexokinase in Y. lipolytica strongly decreased the extent of filamentation for cells growing in fructose-based medium (FIG. 3: B,D,F), and after 5 days of culture, the cells still remained in the yeast form.

In Y. lipolytica and other well-studied yeasts like S. cerevisiae, filamentation is known to be triggered by non-glucose C sources: N acetyl glucosamine in Y. lipolytica (Herrero et al., 1999; Hurtado and Rachubinski, 1999); and mannose, maltose, maltotriose, or sucrose in S. cerevisiae (da Silva et al., 2007; Van de Velde and Thevelein, 2008). Interestingly, both the use of fructose as a carbon source (da Silva et al., 2007) and the absence of hexokinase are also involved in filamentation in S. cerevisiae. In this species, deletion of the gene encoding hexokinase II resulted in the induction of filamentation in a glucose-containing medium (Van de Velde and Thevelein, 2008).

2.4. Hexokinase Overexpression Increases Biomass and Lipid Biosynthesis

In addition to investigating growth, we also compared the lipid production of Y. lipolytica WT strains with that of their corresponding ylHXK1-overexpression transformants; all strains were grown in YNB medium with either 100 g·L⁻¹ glucose or 100 g·L⁻¹ fructose as the carbon source. The C/N molar ratio was fixed at 100 for this experiment. The dry biomass and fatty acids extracted from the cells as well as sugar consumption and citric acid production in the medium were quantified over the 120 h of culture. All the results obtained in this experiment are summarized in Table 6.

TABLE 6
Parameters of fatty acids, biomass and citric acid production by different
origin WT and ylHXK1 transformants of Y. lipolytica growing 120 h in YNB medium
with glucose or fructose (carbon source 100 g · L⁻¹, C/N 100)

| | | Glucose | | | | | | Fructose | | | | | |
|---|---|---|---|---|---|---|---|---|---|---|---|---|---|
| | | W29 | | A-101 | | H222 | | W29 | | A-101 | | H222 | |
| Parameters | | WT | ylHXK1 | WT | ylHXK1 | WT | ylHXK1 | WT | ylHXK1 | WT | ylHXK1 | WT | ylHXK1 |
| X | g · L⁻¹ | 21.4 | 21.9 | 16.8 | 17.3 | 15.4 | 16.2 | 17.4 | 21.6 | 16.9 | 17.8 | 15.7 | 15.8 |
| Y_{X/S} | g · g⁻¹ | 0.27 | 0.28 | 0.25 | 0.26 | 0.23 | 0.26 | 0.25 | 0.28 | 0.27 | 0.27 | 0.23 | 0.25 |
| FA | g · L⁻¹ | 2.57 | 3.28 | 3.03 | 3.12 | 1.69 | 1.95 | 1.56 | 3.02 | 2.19 | 2.85 | 1.26 | 1.90 |
| Y_{FA/X} | g · g⁻¹ | 0.12 | 0.15 | 0.18 | 0.18 | 0.11 | 0.12 | 0.09 | 0.14 | 0.13 | 0.16 | 0.08 | 0.12 |
| Y_{FA/S} | g · g⁻¹ | 0.032 | 0.044 | 0.045 | 0.046 | 0.025 | 0.032 | 0.022 | 0.039 | 0.035 | 0.043 | 0.019 | 0.029 |
| CA | g · L⁻¹ | 0.54 | 2.21 | 4.89 | 8.76 | 1.04 | 0.51 | 0.33 | 1.16 | 2.47 | 3.65 | 0.26 | 0.00 |

Symbols:
X—dry biomass,
FA—fatty acids,
CA—citric acid,
Y_{X/S}—yield of biomass from consumed substrate,
Y_{FA/S}—yield of fatty acids from consumed substrate,
Y_{FA/X}—yield of fatty acids from dry biomass;
SD of all analyzed parameters did not exceed 7%.

The greatest amount of dry biomass, ˜21.5 g·L⁻¹, was obtained from W29 and its ylHXK1 transformant when they were grown on glucose. Lower values were obtained from A-101 and H222 (17 g·L⁻¹ and 16 g·L⁻¹, respectively). The differences in final biomass observed among these strains in flask culture compared to Biotek culture may possibly result from the higher concentration of the carbon source in the flasks and differences in oxygenation between the two systems, which again confirms that physiological differences existed among the strains examined. Overexpression of ylHXK1 did not lead to a significant increase in biomass in YNB glucose medium for any of the strains. In contrast, ylHXK1 overexpression in strain W29 had a large impact on biomass production when the yeast was grown in fructose relative to what was seen for its WT parent, which had been the slowest-growing WT strain in that medium. WT W29 produced around 4 g·L⁻¹ less biomass in fructose than in glucose, while the W29 ylHXK1 transformant yielded similar amounts of dry biomass regardless of the medium's carbon source. Strains A-101 and H222 generated similar amounts of biomass when grown in fructose versus glucose, regardless of whether the hexokinase gene was overexpressed or not.

Slightly less dramatic differences were observed among the different Y. lipolytica strains when biomass yield per unit of substrate consumed was examined (Table 6). The highest value, Y_{X/S}=0.28, was produced by the ylHXK1 transformant of W29 in both substrates. However, a small increase was also observed for the ylHXK1 transformant of H222 relative to its WT parent in both glucose- and fructose-based media. No difference was observed between the A-101 strain and its ylHXK1 transformant in both sugars.

Among the WT strains, A-101 grown in the glucose-based medium yielded the highest amount of total fatty acids (3.03 g·L⁻¹, Table 6) out of all the strain/media combinations that were tested. Although the amount of FAs produced by WT A-101 was lower when the strain was grown in the fructose medium (38% less than in the glucose medium), it was still 74% and 40% higher than the amount obtained in the same medium for the H222 and W29 strains, respectively. Overexpression of hexokinase improved FA production in both substrates for all three Y. lipolytica transformant strains. Although the strain/medium combination that yielded the greatest amount of FAs was the W29 ylHXK1 transformant grown in YNB glucose medium (3.28 g·L⁻¹), the largest increase compared to WT was observed in the same strain in YNB fructose medium (3.02 g·L⁻¹); ylHXK1 overexpression almost doubled the amount of FAs produced as compared to the W29 WT. For the other strains, the effect of hexokinase overexpression on FA production was also more visible when the strains were grown in YNB fructose versus glucose medium; FA production increased by 51% and 30% for H222 and A-101, respectively.

However, when we adjusted these values to account for yeast biomass, we found that the best FA producer, in terms of FA yield obtained per unit biomass, was A-101 grown in YNB glucose medium (0.18 g·g⁻¹, Table 6). This strain produced 63% and 50% more FA per g of biomass than did H222 and W29, respectively. For all the strains, a lower amount of total FAs was produced and the yield of FAs per unit biomass was also lower in YNB fructose than in YNB glucose. Similarly, A-101 was also the best FA producer in the fructose medium, with a yield of 0.13 g of FA per g of biomass compared to yields of 0.09 g·g⁻¹ and 0.08 g·g⁻¹ for W29 and H222, respectively. In terms of production in fructose, the overexpression of hexokinase improved FA yield as compared to the WT for all the transformant strains, but we did not observe large differences in FAs per unit biomass between the WT and ylHXK1 mutants grown in the YNB glucose medium. Hexokinase overexpression resulted in an increase in FA production per g biomass in YNB fructose of 55%, 50%, and 23% for W29, H222, and A-101, respectively. A similar pattern was also observed for measurements of the conversion of consumed substrate into FAs (Y_{FA/S}; Table 6).

In addition to triggering lipid production, nitrogen limitation in Y. lipolytica also results in the production of citric acid (CA). Under the conditions present in this study, low amounts of CA, which is an undesirable by-product of lipid accumulation, were produced. The highest amount of CA was produced by A-101 and its ylHXK1 transformant (Table 6). These two strains produced more than 0.05 g of CA per g of cells, with the A-101 ylHXK1 transformant generating up to 0.13 g per g of YNB glucose substrate (data not shown). In batch culture and under conditions optimized for CA production, WT A-101 was able to produce 0.45 g of CA per gram of glucose (Rywińska et al., 2010). In the W29 and A-101 transformants, the overexpression of ylHXK1 significantly increased CA production in both glucose and fructose media. In contrast, ylHXK1 overexpression in H222 had the opposite effect—CA production was reduced in the glucose medium and absent in the fructose medium.

The three Y. lipolytica WT strains also differed in the composition of the FAs they produced (Table 7). Each strain generated high amounts of C18:1 and C16:0 in both YNB glucose and fructose media, with strain A-101 showing the highest quantity of C18:1 and the lowest quantity of C18:0 and C16:0 compared to the other strains. This result suggests that FA elongation and desaturation in Y. lipolytica A-101 were more efficient than in the other two strains, due to either an increase in activity of the Δ9-desaturase and elongase enzymes or increased stimulation by their respective promoters. A difference was also observed between strains W29 and H222. Although both strains generated similar amounts of C16:0, strain W29 produced more C18:1 than did strain H222; conversely, H222 contained more C18:0 than did W29. This pattern held regardless of whether the carbon source was glucose or fructose. However, both strains contained a higher percentage of C18:2 when grown in YNB fructose than when grown in YNB glucose. Overexpression of ylHXK1 had the clearest impact on fatty acid composition in strain W29 in both the glucose- and fructose-based media. Compared to the composition found in the WT strain, the percentage of C18:1 decreased for C18:0 and slightly for C16:0 in YNB glucose and even more in YNB fructose. It is possible that faster FA synthesis might have resulted in the saturation of Δ9-desaturase and thus reduced the conversion of C18:0 into C18:1. In the other two strains, hexokinase overexpression did not result in any visible changes in relative FA composition.

TABLE 7
Composition of FA produced by Y. lipolytica WT and ylHXK1 transformants
growing 120 h in YNB glucose or fructose medium (carbon source 100 g · L⁻¹, C/N 100)

| | Glucose | | | | | | Fructose | | | | | |
|---|---|---|---|---|---|---|---|---|---|---|---|---|
| Fatty | W29 | | A-101 | | H222 | | W29 | | A-101 | | H222 | |
| acid | WT | ylHXK1 | WT | ylHXK1 | WT | ylHXK1 | WT | ylHXK1 | WT | ylHXK1 | WT | ylHXK1 |
| C16:0 | 17.4 | 18.4 | 11.9 | 11.3 | 18.8 | 17.2 | 15.5 | 18.7 | 12.2 | 12.0 | 17.3 | 16.8 |
| C16:1 | 7.1 | 6.2 | 7.6 | 8.7 | 5.7 | 6.1 | 7.0 | 6.7 | 7.9 | 8.6 | 7.2 | 7.5 |
| (n-7) | | | | | | | | | | | | |
| C18:0 | 11.9 | 13.7 | 8.2 | 7.5 | 15.8 | 14.0 | 9.5 | 13.5 | 7.6 | 7.6 | 12.1 | 11.5 |
| C18:1 | 52.5 | 47.1 | 61.1 | 62.9 | 47.9 | 50.2 | 54.2 | 47.4 | 60.3 | 61.6 | 48.9 | 49.0 |
| (n-9) | | | | | | | | | | | | |
| C18:2 | 7.3 | 8.7 | 7.6 | 6.4 | 7.3 | 7.6 | 9.6 | 8.7 | 8.3 | 6.8 | 10.2 | 10.3 |
| (n-6) | | | | | | | | | | | | |
| Others | 4.0 | 5.9 | 3.6 | 3.3 | 4.5 | 4.8 | 4.2 | 5.0 | 3.7 | 3.4 | 4.3 | 4.9 |

2.5. Impact of Different Hexokinase Genes and Varying C/N Ratios on Fatty Acid and Citric Acid Production

Taking into account all of these results, the French strain W29 was chosen for further analysis, as overexpression of hexokinase in this background resulted in the highest degree of improvement in the parameters examined. Furthermore, many studies on lipid biosynthesis in Y. lipolytica have been performed using W29-derived strains, which made it easier to compare our results on sugar utilization improvement with those from the literature.

Data from the literature regarding the regulation of glycolysis and the characterization of hexokinase reveal differences in the enzyme's kinetics among different organisms. For example, hexokinase in Y. lipolytica is unique in that it is highly sensitive to trehalose-6-phosphate inhibition, much more so than Hxk2p in S. cerevisiae (Petit and Gancedo, 1999). Because of this, we wanted to compare the improvement in FA production resulting from the expression of HXK2 in a Y. lipolytica background (strain PO1d) with that resulting from overexpression of the native Y. lipolytica hexokinase (again in strain PO1d); each inserted gene was regulated by the constitutive TEF promoter (FIG. 7). After 72 h of culture in YNB medium with 60 g·L⁻¹ of fructose, the ylHXK1-overexpression transformant yielded over 80% more fatty acids than did the WT, whereas the strain expressing the scHXK2 gene accumulated 50% more lipids than did the WT. These results reveal that the native hexokinase of Y. lipolytica is more efficient than the one found in S. cerevisiae when it comes to sugar phosphorylation and lipid production from fructose.

As a second test, the effect of varying C/N molar ratios in the YNB fructose medium on lipid production was investigated (FIG. 4A). We found that FA yield differed significantly for different C/N ratios, but that the Y. lipolytica WT and the ylHXK1 transformant responded similarly to each ratio tested. As the C/N ratio increased, the yield of FA from both strains also increased; when the C/N ratio reached 60, however, yields from the WT strain plateaued and those from the ylHXK1 transformant decreased slightly. The highest yield of FA per g biomass was produced with a C/N ratio=60, and it was more than 0.07 g·g⁻¹ higher than for C/N=30. The largest improvement in yield between the WT strain and its ylHXK1 transformant was generated at a C/N ratio of 90. Additionally, after 120 h of culture, the remaining concentration of fructose in the medium was 10, 35, and 62 g·L⁻¹ for C/N ratios of 60, 90, and 120, respectively (FIG. 8C). After two additional days, 23 and 54 g·L⁻¹ of fructose remained in the medium for C/N ratios of 90 and 120, respectively. Under these conditions, the W29 WT strain produced large amounts of citric acid (Table 8), yielding 0.49 and 0.53 g CA per g of substrate consumed at C/N ratios of 90 and 120, respectively. In contrast, under the conditions used in this experiment, hexokinase overexpression significantly decreased CA production at all C/N ratios.

TABLE 8
Parameters of biomass and citric acid production by Y. lipolytica Y3573 in
YNB medium with fructose with different C/N ratio and in rich medium YP with
different fructose concentration.

| | | | C/N ratio | | | | | Fructose concentration (g · L⁻¹) | | | | |
|---|---|---|---|---|---|---|---|---|---|---|---|---|
| Parameter | | | 15 | 30 | 60 | 90 | 120 | 10 | 50 | 100 | 200 | 250 |
| X | g · L⁻¹ | W29 | 4.9 | 12.7 | 14.3 | 14.8 | 14.8 | 7.8 | 31.5 | 46.3 | 61.0 | 62.3 |
| | | ylHXK1 | 7.3 | 12.0 | 21.0 | 21.6 | 21.5 | 9.8 | 35.1 | 49.7 | 56.7 | 58.7 |
| Y_{X/S} | g · g⁻¹ | W29 | 0.36 | 0.47 | 0.33 | 0.30 | 0.29 | 0.78 | 0.63 | 0.46 | 0.32 | 0.29 |
| | | ylHXK1 | 0.49 | 0.44 | 0.42 | 0.32 | 0.33 | 0.98 | 0.70 | 0.50 | 0.31 | 0.27 |
| CA | g · L⁻¹ | W29 | 0.62 | 0.70 | 13.97 | 24.52 | 27.75 | 0 | 0 | 0 | 0 | 0 |
| | | ylHXK1 | 0 | 0.15 | 2.89 | 5.11 | 3.99 | 0.18 | 0.07 | 1.25 | 3.71 | 5.50 |
| Y_{CA/X} | g · g⁻¹ | W29 | 0.127 | 0.055 | 0.976 | 1.656 | 1.875 | 0 | 0 | 0 | 0 | 0 |
| | | ylHXK1 | 0 | 0.013 | 0.138 | 0.237 | 0.186 | 0.018 | 0.002 | 0.025 | 0.065 | 0.094 |
| Y_{CA/S} | g · g⁻¹ | W29 | 0.046 | 0.026 | 0.321 | 0.492 | 0.531 | 0 | 0 | 0 | 0 | 0 |
| | | ylHXK1 | 0 | 0.005 | 0.058 | 0.076 | 0.061 | 0.018 | 0.001 | 0.013 | 0.020 | 0.026 |

Symbols:
X—dry biomass,
CA—citric acid,
Y_{X/S}—yield of biomass from consumed substrate,
Y_{CA/S}—yield of CA from consumed substrate,
Y_{CA/X}—yield of CA from dry biomass

Further analysis of FA production was performed using different fructose concentrations in the presence of 10 g·L⁻¹ of peptones, which are routinely added to Y. lipolytica media to improve growth (FIG. 4B). The aim was to determine the concentration of this sugar that had an optimal effect on lipid biosynthesis without having a negative impact on the cells in terms of increase in osmotic pressure. As in our previous experiments, the observed patterns of FA yield from the Y. lipolytica WT strain and its ylHXK1 transformant were similar, except that, at very high fructose concentrations (over 200 g·L⁻¹), the WT strain stopped accumulating FA while the transformant strain continued. Between the WT and its ylHXK1 transformant, the highest degree of improvement in FA yield from dry biomass (53%) was observed at a fructose concentration of 100 g·L⁻¹. Despite the smaller differences in FA yield between these two strains observed at higher fructose concentrations, the largest overall amount of FAs (0.125 g·g⁻¹) was obtained from a fructose concentration of 250 g·L⁻¹ (FIG. 4B). As in the analysis of different C/N ratios, here we also measured residual fructose in the culture medium (FIG. 8).

After the yeast had spent 120 h in 200 and 250 g·L⁻¹ fructose, 26 and 48 g·L⁻¹ of fructose remained in the medium, respectively, and after an additional 2 days of culture, the remaining sugar concentration was 19 and 36 g·L⁻¹, respectively. Very little CA production was observed in this experiment (Table 8); even at the highest initial fructose concentrations, the yield of CA per unit substrate consumed only reached 2% and 2.6% (for 200 and 250 g·L⁻¹ of initial fructose, respectively). The results obtained for FA production and sugar utilization, taken together with the length of culture in each experiment, suggest that a carbon source concentration of between 100 and 200 g·L⁻¹ is the most promising for the optimization of lipid biosynthesis. This value was subsequently used for experiments involving bioreactor cultures.

2.6. Effects of ylHXK1 Overexpression in a Strain Optimized for Fatty Acid Accumulation

Finally, we investigated the impact of ylHXK1 overexpression in a highly modified strain of Y. lipolytica W29 that was engineered to optimize its oil-production potential; these experiments were conducted in YNB medium containing 60 g·L⁻¹ of carbon source, with a C/N ratio of 60 in order to maximize FA yield (as shown in the previous subsection; for details, see Materials a Methods). As a first step, the genes that encode acyl-coenzyme A oxidases (P0X1-6 genes) were deleted (Beopoulos et al., 2008); the resulting strain had an impaired ability to mobilize accumulated lipids through peroxisomal β-oxidation. In Y. lipolytica, accumulated lipids are stored in specialized organelles called lipid bodies, mainly in the form of triacylglycerols (TAGs) (Daum et al., 1998; Mlickova et al., 2004; Athenstaedt et al., 2006). Fatty acids stored as TAGs can later be efficiently used by the cell through the activity of a lipase attached to the lipid bodies, which is encoded by ylTGL4 (Dulermo et al., 2013). A deletion of this gene was introduced in the Δpox1-6 background to inhibit TAG degradation. Additionally, to increase TAG biosynthesis, the major acyl-CoA: diacylglycerol acyltransferase-en coding gene (ylDGA2) was overexpressed (Beopoulos et al., 2012). Finally, ylGPD1 was overexpressed in order to increase production of glycerol-3-phosphate (Dulermo and Nicaud, 2011); the resulting strain was designated Y3501. All these modifications were then combined with hexokinase overexpression in order to optimize fructose utilization for lipid production. As one of the cheapest fructose-containing substrates is sucrose, we further modified this strain in order to enable utilization of this compound through extracellular hydrolysis, by introducing into the genome a modified cassette for the efficient expression of the S. cerevisiae invertase gene (Lazar et al., 2013). The strain resulting from all of these modifications was called strain Y4086 (Table 4).

To test lipid biosynthesis, batch cultures were grown in non-baffled Erlenmeyer flasks in YNB media that contained 60 g·L⁻¹ of glucose, fructose, or sucrose as a carbon source at a C/N ratio of 60. Strain Y4086 produced around 15 g·L⁻¹ of dry biomass in the sucrose-based medium, which was the highest concentration of biomass generated in this experiment (Table 9). The same strain produced almost 4 g·L⁻¹ less biomass following cultivation in glucose or fructose. This result is probably due to the lower osmotic pressure in sucrose-based media, which allows cells to better adapt to culture conditions (Lazar et al., 2011). Strain Y4086 grown in the sucrose-based medium also generated the highest yield of biomass per unit substrate consumed; it was at least 50% higher than the yield obtained from the same strain grown in YNB medium containing either of the monosaccharides (Table 9).

TABLE 9
Parameters of FA, biomass and CA production of 96 h flask culture
using Y. lipolytica Y3501 and Y4086 strains growing in YNB medium
with glucose, fructose or sucrose (carbon source 60 g · L⁻¹, C/N 60)

| | | Glucose | | Fructose | | Sucrose |
|---|---|---|---|---|---|---|
| Parameters | | Y3501 | Y4086 | Y3501 | Y4086 | Y4086 |
| X | g · L⁻¹ | 11.4 | 11.0 | 10.7 | 11.3 | 15.1 |
| Y_{X/S} | g · g⁻¹ | 0.19 | 0.18 | 0.19 | 0.20 | 0.30 |
| FA | g · L⁻¹ | 3.20 | 2.76 | 2.26 | 2.58 | 4.43 |
| Y_{FA/X} | g · g⁻¹ | 0.281 | 0.250 | 0.212 | 0.228 | 0.294 |
| Y_{FA/S} | g · g⁻¹ | 0.053 | 0.046 | 0.041 | 0.045 | 0.087 |
| CA | g · L⁻¹ | 0.25 | 0.18 | 0.18 | 0.27 | 1.00 |

Symbols:
X—dry biomass,
FA—fatty acids,
CA—citric acid,
Y_{X/S}—yield of biomass from consumed substrate,
Y_{FA/S}—yield of fatty acids from consumed substrate,
Y_{FA/X}—yield of fatty acids from dry biomass.
SD of all analyzis did not exceed 5%.

Strain Y4086 grown in sucrose also produced the largest overall amount of FAs, as well as the best yield per unit biomass (4.43 g·L⁻¹ and 0.294 g·g⁻¹, respectively; Table 9). The same strain grown in YNB medium with either glucose or fructose produced significantly lower concentrations of lipids and lower yields. Additionally, in YNB fructose, only a very small improvement was observed in FA yield from biomass for strain Y4086 as compared to strain Y3501, whereas in YNB glucose, Y4086 actually performed worse in terms of Y_{FA/X} than did Y3501 (Table 9). Thus, the significant improvement in lipid accumulation that we observed in the fructose-based medium between WT W29 and its ylHXK1 transformant (Y3573)—the overexpression transformant contained 74% more FAs—was not repeated by the highly modified Y4086 strain (which only produced 14% more FAs than did Y3501). In this case, it seems that lipid metabolism was limited by another factor that remains to be identified.

The sucrose-based medium also proved itself superior in terms of FA yield per unit substrate consumed (Table 9). The value for Y. lipolytica Y4086 grown in this medium, 0.087 g·g⁻¹, was almost twice as high as that for cultures for which glucose or fructose was the sole carbon source (0.046 and 0.045, respectively). No significant differences were observed in FA yield for strain Y4086 grown in glucose versus fructose media; however, it is worth mentioning that the parental strain Y3501 produced 30% more FAs per unit substrate consumed in glucose-based medium than in fructose-based medium.

No significant concentrations of citric acid were observed at the end of the culture period for either of the Y. lipolytica strains (Table 9).

No significant differences were observed between the FA profiles of Y4086 and Y3501, but slight differences were observed between cultures of the same strain grown in YNB glucose versus fructose (Table 10). However, a comparison of Y4086 and Y3501 with W29 and the W29 ylHXK1 transformant revealed significant differences in the identities of the accumulated FAs (Table 7 and 10). The blocking of β-oxidation and TAG hydrolysis, combined with the increased amount of G3P and TAG synthesis, reduced fatty acid elongation thus increasing C16:0 level in both glucose- and fructose-based media. Therefore meaning amount of C16:0 synthesized in the cytosol could be directly esterified into TAG and would not follow further elongation and desaturation in the endoplasmic reticulum.

TABLE 10
Fatty acid composition in Y. lipolytica strains growing
96 h in YNB medium with glucose, fructose
or sucrose (carbon source 60 g · L⁻¹, C/N 60)

| | Glucose | | Fructose | | Sucrose |
|---|---|---|---|---|---|
| Fatty acid | Y3501 | Y4086 | Y3501 | Y4086 | Y4086 |
| C16:0 | 23.75 | 22.90 | 25.06 | 23.54 | 24.10 |
| C16:1(n-7) | 7.32 | 7.16 | 8.38 | 7.23 | 6.71 |
| C18:0 | 9.86 | 9.98 | 8.23 | 9.49 | 10.00 |
| C18:1(n-9) | 46.79 | 46.87 | 44.72 | 45.67 | 47.82 |
| C18:2(n-6) | 8.23 | 9.07 | 10.10 | 10.21 | 7.60 |
| Others | 4.05 | 4.03 | 3.52 | 3.86 | 3.76 |

2.7. Bioreactor Studies

To investigate strain Y4086 (which had been optimized to produce lipids from sucrose) on a larger scale, bioreactor cultures were grown in YNB sucrose medium (FIG. 5). A study of the improved expression cassette with invertase that was used here has already been published (Lazar et al. 2013); however, here we used the reference strain Y3501 as a control to compare the synergistic effects of the sucrose-hydrolyzing enzyme and sugar hexokinase (sugar phosphorylation). Over the 24 h of culture, strain Y4086, which expressed the pTEF-SUC2 version of invertase, hydrolyzed sucrose at a high rate, 5.28 g·L⁻¹·h⁻¹, from the very beginning of the culture period (FIG. 5A). At the same time, concentrations of glucose in the medium decreased as it was utilized for cell growth, whereas levels of fructose in the culture medium increased as a result of the hydrolysis of sucrose. Fructose began to be consumed only when the supply of glucose in the medium was almost exhausted. Over the 96 h of the experiment, strain Y4086 almost completely depleted the available carbon sources, whereas during the same period, Y3501 used only 70% of the available sugars (fructose was left in the culture medium). In contrast to Y4086, the control strain, which contained the inducible pXPR2-SUC2 version of invertase, hydrolyzed sucrose at a slow rate (0.35 g·L⁻¹·h⁻¹) for the first 72 h of culture and simultaneously consumed both the glucose and fructose present in the culture medium (FIG. 5B). After 72 h, sucrose began to be hydrolyzed more rapidly (at a rate of 2.16 g·L⁻¹·h⁻¹), the rate of glucose consumption remained constant, and fructose began to accumulate in the medium. Additionally, in the case of Y3501, sucrose hydrolysis was delayed by 24 h compared to published observations of the invertase-overexpressing strain JMY2529, which had been reported to hydrolyze this disaccharide within 48 h (Lazar et al., 2013).

Strain Y3501 also demonstrated a slower rate of hydrolysis than did JMY2529, as the rate of the latter reached 2.50 g·L⁻·h⁻¹. Similar trends were observed for strain Y4086, in which sucrose hydrolysis was also delayed for 12 h compared to the invertase-overexpressing strain JMY2531, and its hydrolysis rate was likewise slower (in JMY2531 it has been reported to reach 7.63 g·L⁻·h⁻¹; Lazar et al., 2013). This discrepancy could be explained by the high level of genetic modification of Y3501 and Y4086, which may have resulted in these strains having slower metabolisms.

Both of these strains began to grow as soon as bioreactor culturing was initiated (FIG. 5: A, B). The initial growth rate of Y4086 was 0.20 h⁻¹, and it reached the stationary phase after around 60 h of culture.

Conversely, strain Y3501 grew at a rate of 0.18 h⁻¹ and continued to grow until the end of the experiment. The final biomass of both strains was similar, around 34 g·L⁻¹.

As the medium used for the bioreactor studies contained only low levels of nitrogen and nitrogen limitation plays an important role in both lipid accumulation and CA production, the concentration of both metabolites was analyzed. Strain Y4086 began to secrete CA into the medium at a rate of 1.06 g·L⁻·h⁻¹ after 36 h of culture (FIG. 5A). In this experiment, glucose and fructose levels were high in the medium from the beginning and throughout the culture. Strain Y3501 began to secrete CA into the medium after 72 h of growth, at a rate of 0.77 g·L⁻·h⁻¹; this was also the time at which the strain began to hydrolyze sucrose at a faster rate (see above), and thus when the carbon sources available for cell survival started to be in excess (FIG. 5B). A similar situation was observed for lipid accumulation (FIG. 5). Strain Y4086 accumulated these compounds from the very beginning of the experiment, whereas strain Y3501 accumulated FAs very slowly for the first 60 h of culture. As the initial rate of sucrose hydrolysis was much faster in strain Y4086, its cells had the opportunity to store lipids all throughout the culture period, whereas, because strain Y3501's slow sucrose hydrolysis resulted in a lower availability of carbon, this strain used all available sugar to produce biomass rather than FAs.

As was mentioned above, the final biomass of both Y. lipolytica strains did not differ at the end of the experiment (it was around 34 g·L⁻¹ for each; Table S3). However, a comparison of the yield of dry biomass per unit substrate consumed showed that Y. lipolytica Y3501 converted 50% more carbon into biomass than did strain Y4086. The final biomass production of Y. lipolytica Y4086 was 124% higher in the bioreactor culture as compared to the flask culture, likely as a consequence of the controlled bioreactor conditions; however, at the same time, the yield from substrate decreased by 25%. A search of the available literature regarding strains optimized for lipid production revealed that, in a strain of Y. lipolytica that overexpresses ACC1 and DGA1, 28.5 g·L⁻¹ of biomass can be produced using 90 g·L⁻¹ of glucose as a carbon source, with a yield from substrate of around 0.32 g·g⁻¹ (Tai and Stephanopoulos, 2013). Another strain of Y. lipolytica that was highly modified for lipid biosynthesis produced around 20 g·L⁻¹ of biomass using 80 g·L⁻¹ of glucose as a carbon source, with a yield from substrate of 0.25 g·g⁻¹ (Blazeck et al., 2014). These results suggest that, under conditions optimized for Y. lipolytica strain Y4086, a comparable concentration and yield of biomass could be obtained.

Additionally, as noted earlier, cell morphology played an important role in lipid accumulation (FIG. 9). The reduced lipid yield from Y3501 is consistent with the observation that this strain existed in both yeast and hyphal forms, whereas Y4086 remained in the yeast form throughout the culture period. The overexpression of hexokinase in this strain inhibited hyphal growth and also led to the development of larger lipid bodies inside the cells.

Strain Y4086 produced significantly higher amounts of lipids than did strain Y3501 (Table 11). This improvement was seen in the increase of around 60% in the total lipids, FAs, and FA yield per unit biomass. Although the FA yield from biomass generated in the bioreactor cultures was lower than that generated in the flasks (26.2% and 29.4% respectively), the higher amount of biomass present in the bioreactors allowed for the production of almost 4.5 g·L⁻¹ more total lipids. As described by Tai and Setphanopoulos (2013), a “Push and Pull” strategy involving the overexpression of ACC1 and DGA1 generated 0.617 g lipids per g biomass from cultures grown in 90 g·L⁻¹ of glucose. This result is higher than that obtained in the current study for Y. lipolytica Y4086 grown in the sucrose-based medium (0.262 g·g⁻¹; Table 11). However, the results of Blazeck et al. (2014) indicated that sucrose could be a potentially attractive substrate for lipid production. Through Nile red fluorescence measurements, the authors showed that their highly modified PO1f strain had optimal lipid production on glucose- and mannose-containing substrates, and they found similar results to those obtained here for Y. lipolytica W29. The use of fructose as a carbon source decreased lipid production by 35% (Blazeck et al., 2014), and as demonstrated here, W29 was characterized as the weakest of the Y. lipolytica WT strains examined here in terms of fructose utilization. Taken in the context of our results, it is possible that the reduction in lipid production in the fructose medium observed by Blazeck and colleagues derived from problems with hexokinase limitation, as PO1f is a derivative of W29 (Blazeck et al., 2014). Additionally, the strain created by Blazeck et al. (2014), which expresses the invertase gene under the XPR2 promoter, was limited in its ability to utilize sucrose. These two observations indicate that, in order to efficiently produce lipids from sucrose, rapid sucrose hydrolysis is important (and can be obtained by the overexpression of invertase under a TEF promoter), as is fast sugar phosphorylation (obtained via hexokinase overexpression).

TABLE 11
Parameters of Y. lipolytica Y3501 and Y4086 growing in YNB
medium with sucrose during 96 h of the bioreactor process (sucrose
concentration 150 g · L⁻¹, C/N 60)

| Parameters | | Y3501 | Y4086 |
|---|---|---|---|
| X | g · L⁻¹ | 33.88 | 33.89 |
| Y_{X/S} | g · g⁻¹ | 0.36 | 0.24 |
| Lipids | g · L⁻¹ | 5.76 | 9.15 |
| FA | g · L⁻¹ | 5.45 | 8.89 |
| Y_{FA/X} | g · g⁻¹ | 0.161 | 0.262 |
| Y_{FA/S} | g · g⁻¹ | 0.057 | 0.063 |
| CA | g · L⁻¹ | 42.39 | 64.15 |
| Y_{CA/X} | g · g⁻¹ | 1.13 | 1.78 |
| Y_{CA/S} | g · g⁻¹ | 0.44 | 0.46 |

Symbols:
X—dry biomass,
FA—fatty acids,
CA—citric acid,
Y_{X/S}—yield of biomass from consumed substrate,
Y_{FA/S}—yield of fatty acids from consumed substrate,
Y_{FA/X}—yield of fatty acids from dry biomass.
SD of all analyzis did not exceed 5%.

In contrast to the results from the flask cultures, significantly higher amounts of CA were produced by Y. lipolytica strain Y4086 when it was grown in the bioreactors (Table 11). Up to 64 g·L⁻¹ of CA was generated by this strain when it was grown in a bioreactor with 150 g·L⁻¹ of sucrose, while it only produced 1 g·L⁻¹ during the flask experiment in medium containing 100 g·L⁻¹ of the same substrate (Table 9). The concentration of CA produced by strain Y4086 was 50% higher than that produced by strain Y3501, as was the yield from biomass; however, both strains converted similar amounts of sugar into CA (Y_{CA/S}=0.44-0.46; Table 11). The conversion of sucrose into CA by Y4086 was only slightly lower than that by Y. lipolytica invertase-overexpressing strains JMY2529 and JMY2531, in which 0.50 g·g⁻¹ and 0.58 g·g⁻¹ were generated, respectively (Lazar et al., 2013). These results suggest that the parameters for lipid accumulation in bioreactor cultures for Y. lipolytica strain Y4086 remain to be optimized in order to reduce CA production.

### 3. CONCLUSIONS

As a step towards understanding alternative methods of biofuel production, the complex lipid metabolism of Y. lipolytica has become the target of many studies in recent years. In particular, much of this research seeks to decipher the de novo biosynthesis and accumulation of lipids within the cells of Y. lipolytica. In optimizing biolipid production by this yeast, it has become clear that the selection of substrates is of great importance. From an economic point of view, these substrates must be cheap and widely available, and such raw materials are often sought among industrial byproducts. One of these substrates is sucrose (table sugar), which is a major component of molasses. Although WT strains of Y. lipolytica are not able to utilize this saccharide, it has already been shown that genetically engineered strains that express S. cerevisiae invertase are able to use it by breaking it down into its constituent glucose and fructose molecules. In the present study, we investigated another problem connected to proper sucrose utilization, which is that Y. lipolytica strains differ significantly in terms of their ability to utilize fructose. We determined that impaired fructose assimilation in some strains can be successfully eliminated through the overexpression of the native hexokinase gene. This genetic modification improves not only growth and fructose uptake in Y. lipolytica, but also lipid production from fructose. As a result of the increased hexokinase activity, cells remain in yeast form throughout the culture period; transformant strains are thus able to produce bigger lipid bodies and accumulate more lipids than WT strains. In Y. lipolytica, combining hexokinase overexpression with other genetic modifications of the lipid metabolism process enabled the accumulation of 23% of FA from fructose by 1 g of dry biomass. However, the improvement in lipid production in this strain that resulted from hexokinase overexpression was not as dramatic as that observed between the ylHXK1 transformant (modified only in hexokinase expression) and its WT parental strains. This observation indicates that lipid metabolism in the highly engineered strain Y4086 encountered another limiting factor besides hexokinase activity, and this factor remains to be identified. Additionally, higher lipid accumulation was achieved when sucrose was used as a carbon source instead of its constituents (glucose and fructose); bioreactor cultures in the sucrose-based medium generated 9 g·L⁻¹ of lipids. However, the preferential consumption of glucose over fructose remains a limiting factor that must be addressed in order to increase lipid productivity.

### EXAMPLE 2

### 1. MATERIALS AND METHODS

1.1. Strains, Media

Saccharomyces cerevisiae strain deleted for the hexose transporter EBY.VW4000 was used as recipient strain (hxt⁰; CEN.PK2-1 C Δhxt1-17 gal2Δ::loxP stl1Δ::loxP agt1Δ::loxP mph2Δ:loxP mph3Δ::loxP) [Wieczorke et al., 1999]. Transformants containing Y. lipolytica putative transporters and control strains with empty vectors used in this study are listed in Table 12. Strains were grown at 28° C. on minimal media YNB maltose 2% supplemented with Histidine, Leucine, Tryptophan and Uracil when required. YNB medium for S. cerevisiae contained 6.5 g·L-1 yeast nitrogen base (without amino acids and ammonium sulphate, Difco) and 10 g/L of (NH₄)₂SO₄. Tested carbon sources were added as indicated.

1.2. Cloning of Transporter Genes

Potential Yarrowia lipolytica sugar transporters were identified from literature and BLAST search (Altschul et al., 1990). Among them, 24 genes named according to their systematic name in Génolevures database (http://gryc.inra.fr/; formerly www.genolevures.org) were amplified using primers listed in Table 13 and genomic DNA from W29 or, H222 and A 101 when indicated (Table 12). PCR fragments were cloned in the centromeric plasmid pRS416 containing the ADH1 promoter (Mumberg et al., 1995), the 2μ plasmid pRS426 containing the ADH1 promoter or pRS426 containing the strong TEF promoter (Mumberg et al., 1995) as indicated in Table 12. Plasmids were introduced into S. cerevisiae strain EBY.VW4000) (hxt⁰ using the LiAc transformation protocol and selected on minimal media YNB maltose 2% supplemented with Tryptophan, Histidine and Leucine. Presence of the corresponding gene in the transformants was verified by PCR.

TABLE 13
Primers used in this study.

| | Gene | Gene | | | |
|---|---|---|---|---|---|
| | systematic | usual | Primer | | |
| SEQ ID | name | name | type * | Primer sequence ** | RE ** |
| 44 | A01958 | | Fwd | GCGCACTAGTATGTTCTGGAAGAACATGAAAAATG | SpeI |
| 45 | | | Rev | GCGCAAGCTTTTAACAATTCTCCACATGAATAACAC | HindIII |
| 46 | A08998 | | Fwd | GCGCACTAGTATGAAGCTGTTTAAACGAGAAGC | SpeI |
| 47 | | | Rev | GCGCAAGCTTCTATCCACGAATAGTGGCACCTC | HindIII |
| 48 | A14212 | | Fwd | GCGCACTAGTATGTCAATCAAGTCGCTCTCAAAGG | SpeI |
| 49 | | | Rev | GCGCAAGCTTCTAGACACCATCTTTAGCAACCTTC | HindIII |
| 50 | B00396 | | Fwd | GAGAACTAGTATGTCGCACCGGCCCTGG | SpeI |
| 51 | | | Rev | GCGCAAGCTTTCACCTATCAGCATTTTCACCCATTTCC | HindIII |
| 52 | B01342 | YHT5 | Fwd | GCGCACTAGTATGTACAAGGTCCATAACCCCTACCTC | SpeI |
| 53 | | | Rev | GCGCAAGCTTTTAGACATGCTCAGTTCCAGGATAC | HindIII |
| 54 | B06391 | YHT6 | Fwd | GCGCACTAGTATGATTGGAAACGCTCAAATTAACC | SpeI |
| 55 | | | Rev | GCGCAAGCTTTTACAATTGAGAGGGAGGGGCGTCG | HindIII |
| 56 | B17138 | | Fwd | GCGCACTAGTATGAAAGACTTCCTCGCCTTCAC | SpeI |
| 57 | | | Rev | GCGCAAGCTTCTACGCTGTCTCGATTCGAAC | HindIII |
| 58 | B21230 | | Fwd | GCGCACTAGTATGTCGTCTATATCTTCGTCCCAGCAG | SpeI |
| 59 | | | Rev | GCGCAAGCTTCTACATGGTCCAAACCTCGGTAAAATTT | HindIII |
| | | | | CG | |
| 60 | C04686 | | Fwd | GCGCACTAGTATGTCGCTGGCTATCACCAAC | SpeI |
| 61 | | | Rev | GCGCAAGCTTTTAAGCTGGCTGAGTAGTGTTATTGG | HindIII |
| 62 | C04730 | | Fwd | GCGCACTAGTATGGGCTTCAGAGGCCAAAGAC | SpeI |
| 63 | | | Rev | GCGCAAGCTTTTAAACATGTCTGGTTTCCTCTTGATCA | HindIII |
| | | | | GAAG | |
| 64 | C06424 | YHT1 | Fwd | GCGCACTAGTATGGGACTCGCTAACATCATC | SpeI |
| 65 | | | Rev | GCGCAAGCTTCTAGACAGACTCAATGTAGACTGTCTGT | HindIII |
| | | | | CC | |
| 66 | C08943 | YHT2 | Fwd | GCGCGGATCCATGGCCATTATTGTGGCTGTATTTG | BamHI |
| 67 | | | Rev | GCGCATCGATCTAATCCGAATCAAATCCAGAATCG | ClaI |
| 68 | C16522 | | Fwd | GCGCACTAGTATGAAGCTACAAGTACCCGCGTTTG | SpeI |
| 69 | | | Rev | GAGAGTCGACTCACTGAAACTCGGCCGAATC | SalI |
| 70 | D00132 | | Fwd | GCGCACTAGTATGGTTTTTGGACGAGAAAAAGAC | SpeI |
| 71 | | | Rev | GCGCAAGCTTTTAAACGAACTCGGCAGTG | HindIII |
| 72 | D00363 | | Fwd | GCGCACTAGTATGTTCTGGAAAAACATGAAGAATGAG | SpeI |
| 73 | | | Rev | GAGAGTCGACCTAACAGGTCTCCACGTGAAC | SalI |
| 74 | D01111 | | Fwd | GCGCACTAGTATGGGACGAAACTGGCTAG | SpeI |
| 75 | | | Rev | GCGCCCCGGGTTAAGCTTGAGAAACGTTCTCAAAAG | XmaI |
| 76 | D18876 | | Fwd | GCGCACTAGTATGTTCTGGAAAAATATGAAGAATG | SpeI |
| 77 | | | Rev | GCGCAAGCTTCTAACACGACTCCACCATC | HindIII |
| 78 | E20427 | | Fwd | GCGCACTAGTATGTCCGGGCAGACATATATAG | SpeI |
| 79 | | | Rev | GAGAGTCGACCTAGCAGTTCTCCACATGG | SalI |
| 80 | E23287 | YHT4 | Fwd | GCGCACTAGTATGGCGAGGCTTTGTCTTTC | SpeI |
| 81 | | | Rev | GCGCAAGCTTTTAAACAGTCTCGGTGTACTGAGG | HindIII |
| 82 | F06776 | | Fwd | GCGCACTAGTATGTTTTCGTTAACGGGCAAACC | SpeI |
| 83 | | | Rev | GCGCAAGCTTTTATACCGGAGGTTGAGGGAAGTC | HindIII |
| 84 | F18084 | | Fwd | GCGCACTAGTATGTCTTCCTATCCATCCGAGAAG | SpeI |
| 85 | | | Rev | GAGTAAGCTTTTAAGCAAGCTCCGCCGTGTG | HindIII |
| 86 | F19184 | YHT3 | Fwd | GCGCGGATCCATGTCCACTAGTGCTATGAC | Bann HI |
| 87 | | | Rev | GCGCAAGCTTCTAAGAGGACTCGGAGAAGTC | HindIII |
| 88 | F23903 | | Fwd | GCGCGGATCCATGTCGCTGGACAAAAACC | BamHI |
| 89 | | | Rev | GCGCAAGCTTCTACTTCTTGTAGCCTCTCTTGG | HindIII |
| 90 | F25553 | | Fwd | GCGCACTAGTATGATACTTTTTTGGTTACACAGAGGCG | SpeI |
| | | | | TCTTC | |
| 91 | | | Rev | GCGCAAGCTTTTATTGATGAGTGGTGGTGTCGGGGTA | HindIII |
| | | | | C | |
| 92 | C06424- | YHT1_{H}- | Fwd | CAGTTTGCCGTCACCATTGGTCTTCTGC | I162V |
| 93 | I162V | 162V | Rev | GCAGAAGACCAATGGTGACGGCAAACTG | |
| 94 | F19184- | YHT3_{H}- | Fwd | CCAGCTGTTTGTTACTCTCGGCATCTTC | I181V |
| 95 | I181V | 181V | Rev | GAAGATGCCGAGAGTAACAAACAGCTGG | |

* Abreviations: Fwd: forward; Rev: revese.
** Restriction site introduced for cloning are undelined and base changes to introduce the mutation for the amino acid exchange are boled. To simplify in the table the systematic name for the putative transporter were names according to Génolevures nomenclature (Durrens, P., Sherman, D. J. (2005)) without YALI0, e.g. YALI0A01958 are named A01958. YHT genes were amplified from the Y. lipolytica wild-type W29 or from the wild-type H222 for the variants of the isoleucine 162/181 altered to valine for YHT1_{H}-162V and YHT3_{H}-181V, respectively.

1.3. Site-Directed Mutagenesis.

Mutations were inserted into H222 C06424 (YHT1_{H}) and F19184 transporter (YHT3_{H}) by site-directed PCR mutagenesis. First, 5′ and 3′ fragments (Yhtx-a and Yhtx-b, respectively) were amplified with primer pairs Fwd/Mut-rev and Mut-fwd/Rev, respectively, using the Pyrobest polymerase (TaKaRa). The two mutagenesis primers covered the target codon and the neighboring 15-20 nucleotides and were directed in opposing directions (forward and reverse). The two fragments were then used as templates in PCR fusion with flanking primers pairs fwd/rev to produce the full-length YHT variant (Table 13).

YHT1_{H}-161V allele encoding the mutated C06424 gene from H222 for the Isoleucine 161 was constructed as follows. First, YHT1-a and YHT1-b fragments were amplified with primer pairs (C06424-fwd/C064241161Vmut-rev) and (C06424-rev/C064241161Vmut-fwd). The second PCR fusion contained the two fragments with primer pair C06424-fwd/C06424-rev to produce the full-length YHT1_{H}-161V allele. YHT3_{H}-181V allele encoding the mutated F19184 gene for the Isoleucine 181 was amplifies similarly with specific primers (Table 13) giving rise to the YHT3_{H}-181V allele.

1.4. Sugar Utilisation Test.

The S. cerevisiae transformants were grown at 30° C. for 24 h in the minimal media YNB maltose 2% three time successively in order to increase and standardize the plasmid copy number. For drop test, exponentially growing cells were centrifuged, washed twice with water and re-suspended to an optical density OD₆₀₀ of 1. Successive 10-fold dilutions were performer (10⁰-10⁻⁵) and 5 μl of each dilution were spotted onto YNB plate containing various sugar (glucose, fructose, mannose and galactose) and at different concentration (0.1 to 2% as indicated in the text or in figure legend).

1.5. Growth in Microtiter Plates

Yeast strains were grown in 96-well plates in 200 μl of minimal YNB medium containing either 1% glucose, 1% fructose or mixture of glucose and fructose (0,5% each). Precultures were obtained from frozen stocks, inoculated into tubes containing 5 mL YNB maltose 2% medium, and cultured for 24 h (170 rpm, 28° C.). Precultures were then centrifuged washed with sterile distilled water and their concentrations were standardized to an OD₆₀₀ of 0.1. This analysis was conducted three times, with 2-3 replicates per plate for each condition. Cultures were maintained at 28° C. under constant agitation with a Biotek Synergy MX microtiter plate reader (Biotek Instruments, Colmar, France); each culture's optical density at 600 nm was measured every 20 min for 72 h.

1.6. Media and Growth for Sugar Utilization Experiment in Flask

For sugar utilization in minimal media, cultures were prepared as follows: an initial preculture was established by inoculating 50 mL of YNB maltose 2% medium in 250-mL Erlenmeyer flasks; this was followed by an overnight shaking step at 28° C. and 170 rpm. The resulting cell suspension was washed three times with sterile distilled water and used to inoculate 50 mL of the main culture containing 1% of glucose, fructose or mixture of those sugars. Each culture was incubated, with shaking in 250-mL Erlenmeyer flasks, at 28° C. and 170 rpm during 72 h, or until all available sugar had been consumed. Samples for analysis were taken every 12 h.

1.7. Sugar Concentration Measurement

Glucose and fructose were identified and quantified by HPLC (UltiMate 3000, Dionex-Thermo Fisher Scientific, UK) using an Aminex HPX87H column coupled to UV (210 nm) and RI detectors. The column was eluted with 0.01 N H₂SO₄ at room temperature and a flow rate of 0.6 mL·min⁻¹. Identification and quantification were achieved via comparisons to standards. Before being subject to HPLC analysis, samples were filtered on 0.45-μm pore-size membranes.

### 2. RESULTS

2.1. Identification of putative Y. lipolytica hexose transporters.

Yarrowia lipolytica genome of strain E150 was determined 10 years ago by the Génolevures consortium (Dujon et al, 2004). Genome analysis shows that the GL3C0002 family coding for putatif sugar transporters contained 23 members from Yarrowia. This family contains the well known S. cerevisiae Hxt7 hexose transporter.

Little information has been reported for Y. lipolytica transporters (Young et al., 2011; Yong et al., 2014). The only report during this study was from Alper and coworkers which, during a survey on yeast sugar transporter preference, analysed the sugar preference of 6 members of this family, namely B01342, B06391, C06424, C08943, D00132 and F06776. These transporters were expressed in ARS plasmid (p414-TEF, CEN6/ARS4 origin; see Mumberg et al., 1995) under the control of the constitutif TEF promoter and transformed into S. cerevisiae strain EX12. Limited growth was observed only for C06424 with a growth rate depending on sugar tested of about 0.05-0.06 compared to 0.03-0.05 for the empty vector and 0.191, 0.254 and 0.278 for EX12 expressing S. cerevisiae Hxt7 on glucose, fructose and mannose, respectively (Young et al. 2014). Thus suggesting that only the Y. lipolytica C06424 transporter could transport glucose, galactose and manose but not fructose.

2.2. Functional Characterisation of Putative Y. Lipolytica Hexose Transporters in the S. Cerevisiae Heterologous Host by Drop Tests.

For the characterization and screening of putative sugar transporters, the hexose deficient Saccharomyces cerevisiae hxt⁰ strain EBY.VW4000 developed by Boles E. and coworker (Wieczorke et al., 1999) is wildly used. This strain lacks all 20 transporter genes (HXT1-17, GAL2, AGT1, MPHs) required for hexose uptake which prevents growth of glucose, fructose, mannose and galactose, thus allowing assessment of the function of heterologous transporters.

First, among the Y. lipolytica putative transporters, the three closest genes to S. cerevisiae HXT7 transporters, C06424 (YHT1), C08943 (YHT2) and F19184 (YHT3) were amplified from strain W29 and cloned into the replicative ARS plasmid pRS416 under the ADH1 promoter. Only the transformants carrying YHT1 present very slow growth on glucose plate while no growth could be observed for the transformants carrying YHT2 or YHT3 (data not shown) being slightly lower than that observed by the Alper group (Young et al, 2014). In addition no growth was observed for the three genes on fructose. This lack of efficient growth may result from low expression of the Y. lipolytica genes in S. cerevisiae or due to polymorphism in the corresponding gene in strain W29 used for the amplification. Therefore, the genes were amplified also using H222 and A-101 genomic DNA and cloned into 2μ based plasmids pRS426 either under the ADH1 and the TEF promoter. For strain harboring YHT1, the TEF promoter enables better growth than the ADH1 promoter which remain limited (FIG. 10A) while no growth was observed for YHT2 with both promoters on glucose.

Similar promoter-dependent growth was observed on fructose plates. This confirmed that both strong transporter expression and the use of 2μ based plasmid were required to observe growth complementation of S. cerevisiae strain EBY.VW4000.

Since that growth on fructose differed depending on strain origin, we hypothesized that this may also be due to differences of the fructose transport in addition to hexokinase defect. Therefore, the YHT1 and YHT2 genes were also amplified using H222 genomic DNA and YHT3 was amplified from both H222 and A-101 genomic DNA and will be named YHTX_{W}, YHX_{H} and YHTX_{A}, respectively. Growth complementation with the different alleles shown in FIG. 10B showed partial complementation on both glucose and fructose with the YHT3_{W} in contrast to the very efficient growth with strains expressing YHT3_{H} and YHTX_{A}.

Second, we extended the functional analysis by amplification and cloning 20 additional putative transporters from Y. lipolytica W29 and 11 from H222 into the 2μ plasmid pRS426 under the TEF promoter as described in Table 13. The corresponding strains are described in Table 12. Growth of transformants on 2% maltose was tested to verify the absence of growth defect induced by overexpression of hexose transporters (FIG. 11).

Growth of transformants was tested on four sugars; glucose, fructose, mannose and galactose at four different concentrations, 0.1 to 2% (FIG. 12). In this experiment, no growth complementation could be observed on the four sugars whatever the concentration with transporters A08998, C04730, F06776, A14212, C16522 and F25553 (FIG. 12 and data not shown). These results were similar for alleles of both H222 and W29 strains. Thus, indicating that they are not functional or not expressed in S. cerevisiae in our conditions.

Four of them allowed growth on glucose; B01342, C06424, E23287 and F19184 and were designated YHT5, YHT1, YHT4 and YHT3 respectively (Table 1). However, while YHT1_{H} and YHT1_{W} are functional, YHT5 did not allowed efficient growth on glucose and YHT3_{W} requires high glucose concentration for complementation.

The Yht2 transporter allowed growth on fructose, better at low concentration, independently to the allele used. While for the YHT3 transporter, complementation is clearly depending on the allele used, YHT3_{W} confers reduced growth on fructose compared to the YHT3_{H} and YHT3_{A}.

At least one other protein of the putative transporter could sustain hexose transport in the host S. cerevisiae. D01111 was found to complement the HXT-deficient EBY.VW4000 strain only for glucose uptake and resulted in a weak growth compared to that provided by YHT genes.

2.3. Functional Characterisation of Putative Y. Lipolytica Hexose Transporters in the S. Cerevisiae Heterologous Host in Liquid Media.

To further characterize putative Y. lipolytica transporters, growth of transformants was analysed in liquid media in 96 well microplate on glucose, fructose and glucose-fructose mixture. Representative curves for five YHT of Y. lipolytica H222 strain are presented in FIG. 13. No growth could be detected for C04730 and A08998 on any tested sugars,and E23287 presents only a slight growth on mixture of sugars; this latter transporter was designated YHT4. On the other hand YHT1 (C06424) and YHT3 (F19184) present better growth on fructose than on glucose. On mixture of sugars, YHT1 shows average growth, between glucose and fructose, while YHT3 exhibits similar growth to the one on fructose.

2.4. Growth and Sugar Consumption by the S. Cerevisiae Heterologous Host.

Previous report of invertase overexpression in Y. lipolytica shows the preferred consumption of glucose over fructose, suggesting an inhibition of fructose utilisation by glucose (Lazar et al, 2013). Thus growth and sugar consumption was monitored during growth in fructose media depending on glucose concentration in flasks (FIG. 14a).

As for Biotek experiments, no growth was observed for C04730 and A08998. In the case of YHT4 (E23287), the low growth on fructose could be improved by addition of small amounts of glucose, confirming its substrate preference for glucose. By contrast, growth of EBY.VW4000 overexpressing YHT1 (C06424) on fructose is inhibited by increasing amounts of glucose. Additionally, the highest OD₆₀₀ was reached by EBY.VW4000 overexpressing YHT3 (F19184). In all conditions growth profiles are similar.

Several of categories of transporter could be identified by analyzing sugar consumption. In this experiment, A08998 and C04730 showed no capacity to transport glucose or fructose. Yht1 (C06424), Yht4 (E23287), and Yht3 (F19184) are able to uptake glucose and fructose. For the former two, presence of glucose highly delays fructose utilization, whereas YHT3 (F19184) shows only slightly delayed fructose consumption if not concomitant with glucose (FIG. 14b).

The time course of residual sugars in the medium was examined. This reflects the consumption of fructose and glucose for S. cerevisiae cells expressing each of the identified fructose transporters (Yht1 to 4; the most efficient Yht3_{H222} was chosen), grown in the presence of fructose (10 g·L⁻¹) and varying concentrations of glucose.

First, fructose utilization appeared to be impeded by glucose in presence of equal amount of both sugars, whatever the transporter being expressed, including Yht2 which is not able to promote glucose uptake and Yht1 which seems to transport glucose alone less efficiently than fructose. Conversely the presence of fructose did not preclude the uptake of glucose for none of the Yht1, Yht3H222 or Yht4 transporters (Yht2 is not able to internalize glucose).

Second, lowering glucose concentration in the medium (5 g·L⁻¹ or 1 g·L⁻¹ at start of the culture, or in the course of cultivation through glucose consumption) relieved the inhibition exerted on fructose consumption. When Yht4 was expressed, this relief occurred for a remaining glucose concentration under about 0.8 g·L⁻¹. For Yht1, slopes of residual fructose in the medium actually suggest that glucose may be competing with fructose uptake in a rate positively proportional to its concentration in the medium rather than inhibitory in a threshold manner. Consumption of fructose by Yht3-expressing cells is unique since it is merely slightly delayed in the presence of external glucose over 4-5 g·L-land no competition with glucose was evidenced below this concentration.

### 3 FUNCTIONAL ANALYSIS IN Y. LIPOLYTICA

3.1 Deletion Analysis of YHT Genes

To identify the main transporters involved in growth of Y. lipolytica on fructose, derivatives of W29 carrying individual disruptions of the YHT genes promoting fructose transport (YHT1 to 4) or combination thereof, were constructed. Growth tests were performed in a microplate reader or as drop-test assays on plates in YNB minimal medium supplemented with individual sugars (fructose, glucose and mannose; galactose was not tested due to the inability of WT Y. lipolytica to grow on this sugar).

The single yht1 mutant displayed a significant phenotype in fructose. At 1 g·L⁻¹ of fructose, the sole YHT1 disruption was sufficient to prevent growth of Y. lipolytica, showing the essential role of this single gene in the uptake of fructose at low concentration. At 10 g·L⁻¹, an unexpected phenotype was observed, as the yht1 mutant grew more robustly than the WT strain. No particular phenotype was observed in glucose or mannose. Transformation of yht1Δ by YHT1 restored WT growth on fructose.

Deletion of YHT2 or YHT3 alone had no detectable effect on growth, neither in fructose nor in glucose. Moreover, strains carrying double mutations of YHT2 and YHT1 or of YHT3 and YHT1 showed the same phenotypes as the single yht1Δ mutant.

Likewise, the single yht4Δ mutant exhibited no significant growth alteration compared to the WT strain. However the combination of this deletion with the yht1 mutation led to a growth defect in fructose, glucose and mannose. The double deletion of YHT1 and YHT4 was sufficient to abolish growth on fructose at all tested concentrations from 1 to 10 g·L⁻¹. Growth on glucose was also severely affected in the double yht1Δ yht4Δ mutant. However, residual growth could sporadically outcome as filamentous-type colonies on YNB glucose plates after incubation for several days as well as very delayed growth in microplates. Moreover growth on mannose was also abolished in the double yht1Δ yht4Δ mutant showing that the two encoded transporters are necessary and sufficient for hexose transport in laboratory conditions for Y. lipolytica.

3.2 Transcription of YHT Genes

The transcription profiles of the YHT genes and D01111 in WT genetic backgrounds were investigated.

A first RT-PCR analysis was carried out during growth of W29 and H222 in minimal medium supplemented with the sole fructose at 1 g·L⁻¹ or 10 g·L⁻¹. Transcription profiles were very similar for both natural isolates (FIG. 15). YHT1 and YHT4 were the only two genes to be consistently transcribed in fructose. Transcripts for YHT5 and D01111 were sporadically detected, possibly indicative of low level of transcripts, whereas transcripts for YHT2, YHT3 and YHT6 were not detected at all. This result is consistent with the gene deletion analysis performed in the W29 context, showing that YHT1 and YHT4 code for the main transporters involved in growth on fructose. These results also indicate that the same two transporters are likely to be the physiologically active ones for H222, although the latter codes for a potentially very active Yht3 transporter for fructose.

In a second analysis, we investigated the transcription of YHT and D01111 genes in the complex environment of a bioreactor in which cells were grown in sucrose medium. The W29 and H222 derivatives used here were equipped with an efficient invertase expression cassette (Lazar et al., 2013). The continuous hydrolysis of sucrose by the secreted enzyme and uptake of the monosaccharides by the cells generated changing concentrations of glucose and fructose in the medium that could be followed by HPLC. This provided an interesting environment to study whether the expected delayed uptake of fructose (in the presence of glucose) could be related to transporter gene expression.

We could observe early raising concentrations of glucose and fructose, due to sucrose hydrolysis faster than uptake of released sugars. The uptake of glucose started from the beginning of cultivation whereas fructose was consumed only after glucose depletion (W29) or shortening (H222).

The transcription profiles, which are similar for the two strains, could be divided into two classes of transporter genes. The first one includes YHT1, YHT4 and D01111 whose transcripts are detected continuously during cultivation. YHT5 could be a particular case whose transcripts although continuously detected, apparently increased at the beginning of stationary phase. The second one comprises YHT2, YHT3 and YHT6 whose transcripts are detected essentially at stationary phase. Altogether transcripts were detected for all 7 genes (YHT1-6 and D01111) in both strains at entry to stationary phase after glucose depletion, transiently or not. This was also true for other genes of the SP family picked at random.

In conclusion, the RT analysis confirmed that Yht1 and Yht4 are major hexose transporters involved in fructose uptake in Y. lipolytica. It suggested that Yht5 and/or D01111 might be responsible for the residual fastidious growth sporadically observed on glucose in the yht1-4 mutant strain. In addition, this analysis showed that inhibition of transcription of transporter gene for fructose uptake is not the molecular basis for glucose over fructose preference. Conversely, the better detection of transcripts of D01111 in the complex bioreactor environment suggests a possible induction of the gene by sucrose or glucose.

### REFERENCES

Altschul S F, Gish W, Miller W, Myers E W, Lipman D J: Basic local alignment search tool. J Mol Biol 1990, 215(3):403-410.

Athenstaedt K, Jolivet P, Boulard C, Zivy M, Negroni L, Nicaud J M, Chardot T. 2006. Lipid particle composition of the yeast Y. lipolytica depends on the carbon source. Proteomics. 6: 1450-1459.

Barth G, Gaillardin C. 1996. Y. lipolytica. In: Wolf K (ed.). Non conventional yeasts in biotechnology, a handbook. Springer. p. 313-388.

Beopoulos A, Mrozova Z, Thevenieau F, Dall M T, Hapala I, Papanikolaou S, Chardot T, Nicaud J M. 2008. Control of lipid accumulation in the yeast Y. lipolytica. Applied Environmental Microbiology. 74: 7779-7789.

Beopoulos A, Cescut J, Haddouche R, Uribelarrea J L, Molina-Jouve C, Nicaud J M. 2009. Y. lipolytica as a model for bio-oil production. Progress in Lipid Research. 48: 375-387.

Beopoulos A, Haddouche R, Kabran P, Dulermo T, Chardot T, Nicaud J M. 2012. Identification and characterization of DGA2, an acyltransferase of the DGAT1 acyl-CoA:diacylglycerol acyltransferase family in the oleaginous yeast Y. lipolytica. New insights into the storage lipid metabolism of oleaginous yeasts. Applied Microbiology and Biotechnology. 93(4): 1523-1537.

Blazeck J, Hill A, Liu L, Knight R, Miller J, Pan A, Otoupal P, Alper H S. 2014. Harnessing Y. lipolytica lipogenesis to create a platform for lipid and biofuel production. Nature Communications. DOI: 10.1038/ncomms4131.

Browse J, McCourt P J, Somerville C R. 1986. Fatty acid composition of leaf lipids determined after combined digestion and fatty acid methyl ester formation from fresh tissue. Analytical Biochemistry. 152: 141-145.

Coelho M A Z, Amaral P F F, Belo I. 2010. Y. lipolytica: an industrial workhorse. In: Méndez-Villas A (ed). Current Research, Technology and Education Topics in Applied Microbiology and Microbial Biotechnology. Vol. 2. Formatex, Badajoz, p 930.

Daum G, Lees N D, Bard M, Dickson R. 1998. Biochemistry, cell biology and molecular biology of lipids of S. cerevisiae. Yeast. 14: 1471-1510.

da Silva P C, Horii J, Santos Miranda V, Gallera Brunetto H, Ceccato-Antonini S R. 2007. Characterization of industrial strains of S. cerevisiae exhibiting filamentous growth induced by alcohols and nutrient deprivation. World Journal of Microbiology and Biotechnology. 23: 697-704.

Dujon B, Sherman D, Fischer G, Durrens P, Casaregola S, Lafontaine I, De Montigny J, Marck C, Neuveglise C, Talla E, Goffard N, Frangeul L, Aigle M, Anthouard V, Babour A, Barbe V, Barnay S, Blanchin S, Beckerich J M, Beyne E, Bleykasten C, Boisrame A, Boyer J, Cattolico L, Confanioleri F, De Daruvar A, Despons L, Fabre E, Fairhead C, Ferry-Dumazet H, Groppi A, Hantraye F, Hennequin C, Jauniaux N, Joyet P, Kachouri R, Kerrest A, Koszul R, Lemaire M, Lesur I, Ma L, Muller H, Nicaud J M, Nikolski M, Oztas S, Ozier-Kalogeropoulos O, Pellenz S, Potier S, Richard GF, Straub M L, Suleau A, Swennen D, Tekaia F, Wesolowski-Louvel M, Westhof E, Wirth B, Zeniou-Meyer M, Zivanovic I, Bolotin-Fukuhara M, Thierry A, Bouchier C, Caudron B, Scarpelli C, Gaillardin C, Weissenbach J, Wincker P, Souciet J L. 2004. Genome evolution in yeasts. Nature. 430(6995): 35-44.

Dulermo T, Nicaud J M. 2011. Involvement of the G3P shuttle and β-oxidation pathway in the control of TAG synthesis and lipid accumulation in Y. lipolytica. Metabolic engineering. 13(5): 482-491.

Dulermo T, Tréton B, Beopoulos A, Kabran Gnankon A P, Haddouche R, Nicaud J M. 2013. Characterization of the two intracellular lipases of Y. lipolytica encoded by TGL3 and TGL4 genes: new insights into the role of intracellular lipases and lipid body organisation. Biochimica and Biophysica Acta. 1831(9): 1486-1495.

Durrens, P., Sherman, D. J. 2005. A systematic nomenclature of chromosomal elements for hemiascomycete yeasts. Yeast 22(5), 337-342.

Dyer J M, Chapital D C, Kuan J W, Mullen R T, Pepperman A B. 2002. Metabolic engineering of S. cerevisiae for production of novel lipid compounds. Applied Microbiology and Biotechnology. 59(2-3): 224-230.

Ernandes J R, De Meirsman C, Rolland F, Winderickx J, de Winde J, Brandao R L, Thevelein J M. 1998. During the initiation of fermentation overexpression of hexokinase PII in yeast transiently causes a similar deregulation of glycolisys as deletion of Tps1. Yeast. 14(3): 255-269.

Fickers P, Le Dall M T, Gaillardin C, Thonart P, Nicaud J M. 2003. New disruption cassettes for rapid gene disruption and marker rescue in the yeast Y. lipolytica. Journal of Microbiological Methods. 55: 727-737.

Fickers P, Nicaud J M, Destain J, Thonart P. 2005a. Involvement of hexokinase Hxk1 in glucose catabolite repression of LIP2 encoding extracellular lipase in the yeast Y. lipolytica. Current Microbiology. 50(3): 133-7.

Fickers P, Benetti P H, Waché Y, Marty A, Mauersberger S, Smit M S, Nicaud J M. 2005b. Hydrophobic substrate utilisation by the yeast Y. lipolytica, and its potential applications. FEMS Yeast Research. 5:527-543.

Folch J, Lees M, Sloane-Stanley G H. 1957. A simple method for the isolation and purification of total lipides from animal tissues. Journal of Biological Chemistry. 226: 497-509.

Gaillardin C, Ribet A M. 1987. LEU2 directed expression of β-galactosidase activity and phleomycin resistance in Y. lipolytica. Current Genetics. 11: 369-375.

Groenewald M, Boekhout T, Neuvéglise C, Gaillardin C, van Dijck P W M, Wyss M. 2014. Y. lipolytica: Safety assessment of an oleaginous yeast with a great industrial potential. Critical Reviews in Microbiology. 40(3): 187-206.

Guillaume C, Delobel P, Sablayrolles J M, Blondin B. 2007. Molecular Basis of Fructose Utilization by the Wine Yeast S. cerevisiae: a Mutated HXT3 Allele Enhances Fructose Fermentation. Applied and Environmental Microbiology, 73(8): 2432-2439.

Gysler C, Kneuss P, Niederberger P. 1990. Transformation of commercial baker's yeast strains by electroporation. Biotechnol Tech 4: 285-290.

Herrero A B, López M C, Fernandez-Lago L, Dominguez A. 1999. Candida albicans and Y. lipolytica as alternative models for analysing budding patterns and germ tube formation in dimorphic fungi. Microbiology. 145: 2727-2737.

Hoess R H, Abremski K. 1985. Mechanism of strand cleavage and exchange in the Cre-lox site-specific recombination system. J Mol Biol. 181(3): 351-362.

Hurtado C A, Rachubinski R A. 1999. MHY1 encodes a C2H2-type zinc finger protein that promotes dimorphic transition in the yeast Y. lipolytica. Journal of Bacteriology. 181: 3051-3057.

Ito H, Fukuda Y, Murata K, Kimura A. 1983. Transformation of intact yeast cells treated with alkali cations. Journal of Bacteriololy. 153: 163-168.

Klebe R J, Harriss J V, Sharp Z D, Douglas M G. 1983. A general method for polyethylene-glycol-induced genetic transformation of bacteria and yeast. Gene. 25(2-3): 333-341.

Kurat et al. 2006. Obese yeast: triglyceride lipolysis is functionally conserved from mammals to yeast. Journal of Biological Chemistry 281: 491-500.

Lazar Z, Walczak E, Robak M. 2011. Simultaneous production of citric acid and invertase by Y. lipolytica SUC⁺ transformants. Bioresource Technology. 102(13): 6982-6989.

Lazar Z, Rossignol T, Verbeke J, Crutz-Le Coq A M, Nicaud J M, Robak M. 2013. Optimized invertase expression and secretion cassette for improving Y. lipolytica growth on sucrose for industrial applications. Journal of Industrial Microbiology and Biotechnology. 40(11): 1273-1283.

Leandro, M. J., Fonseca, C. and Goncalves, P. 2009. Hexose and pentose transport in ascomycetous yeasts: an overview. FEMS Yeast Research, 9: 511-525.

Le Dall M T1, Nicaud J M, Gaillardin C. 1994. Multiple-copy integration in the yeast Yarrowia lipolytica. Curr Genet. 26(1): 38-44.

Liccioli T, Chambers P J, Jiranek V. 2011. A novel methodology independent of fermentation rate for assessment of the fructophilic character of wine yeast strains. Journal of Industrial Microbiology and Biotechnology. 38(7): 833-43.

Mauersberger S, Wang H J, Gaillardin C, Barth G, Nicaud J M. 2001. Insertional mutagenesis in the n-alkane-assimilating yeast Y. lipolytica: generation of tagged mutations in genes involved in hydrophobic substrate utilization. Journal of Bacteriology. 183: 5102-5109.

Madzak C, Tréton B, Blanchin-Roland S. 2000 Strong hybrid promoters and integrative expression/secretion vectors for quasi-constitutive expression of heterologous proteins in the yeast Yarrowia lipolytica. J. Mol. Microbiol. Biotechnol., 2(2): 207-216.

Madzak C, Gaillardin C, Beckerich J M. Heterologous protein expression and secretion in the non-conventional yeast Yarrowia lipolytica: a review. 2004 Journalof Biotechnology, 109(1-2): 63-81.

Maftahi M, Gaillardin C, Nicaud J M. 1996 Sticky-end polymerase chain reaction method for systematic gene disruption in Saccharomyces cerevisiae. Yeast. 12(9): 859-868.

Michely S, Gaillardin C, Nicaud J M, Neuvéglise C. 2013. Comparative physiology of oleaginous species from the Yarrowia clade. PLOS one. 8(5): e63356 (1-10).

Mirończuk A M, Furgata J, Rakicka M, Rymowicz W. 2014. Enhanced production of erythritol by Y. lipolytica on glycerol in repeated batch cultures. Journal of Industrial Microbiology and Biotechnology. 41 (1): 57-64.

Mlickova K, Roux E, Athenstaedt K, d'Andrea S, Daum G, Chardot T, Nicaud J M. 2004. Lipid accumulation, lipid body formation, and acyl coenzyme a oxidases of the yeast Y. lipolytica. Applied and Environmental Microbiology. 70: 3918-3924.

Moeller L, Zehnsdorf A, Aurich A, Barth G, Bley T, Strehlitz B. 2012. Citric acid production from sucrose by recombinant Y. lipolytica using semicontinuous fermentation. Engineering in Life Sciences. 13(2): 163-171.

Müller S, Sandal T, Kamp-Hansen P, Dalbøge H. 1998. Comparison of expression systems in the yeasts S. cerevisiae, Hansenula polymorpha, Kluyveromyces lactis, Schizosaccharomyces pombe and Y. lipolytica. Cloning of two novel promoters from Y. lipolytica. Yeast. 14 (14):1267-1283.

Mumberg D, Müller R, Funk M. 1995. Yeast vectors for the controlled expression of heterologous proteins in different genetic backgrounds. Gene 156(1): 119-122.

Nicaud J M, Fabre E, Gaillardin C. 1989. Expression of invertase activity in Y. lipolytica and its use as a selective marker. Current Genetics. 16(4): 253-260.

Orr-Weaver T L, Szostak J W, Rothstein R J. 1981 Yeast transformation: a model system for the study of recombination. Proc Natl Acad Sci USA. 78(10): 6354-6358.

Petit T, Gancedo C. 1999. Molecular Cloning and Characterization of the Gene HXK1 Encoding the Hexokinase from Y. lipolytica. Yeast. 15: 1573-1584.

Querol A, Barrio E, Huerta T, Ramon D. 1992. Molecular monitoring of wine fermentations conducted by active dry yeast strains. Applied and Environmental Microbiology, 58: 2948-2953.

Reifenberger et al. 1995. Yeast. 11: 5457.

Rywińska A, Rymowicz W. 2010. High-yield production of citric acid by Y. lipolytica on glycerol in repeated-batch bioreactors. Journal of Industrial Microbiology and Biotechnology. 37 (5): 431-435.

Rywińska A, Rymowicz W, Żarowska B, Skrzypiński A. 2010. Comparison of citric acid production from glycerol and glucose by different strains of Y. lipolytica. World Journal of Microbiology and Biotechnology. 26: 1217-1224.

Sambrook J, Russell D W. 2001. Molecular Cloning: A laboratory manual, 3rd ed, Cold Spring Harbor Laboratory Press, New York.

Sauer B. 1987. Functional expression of the cre-lox site-specific recombination system in the yeast Saccharomyces cerevisiae. Mol Cell Biol. 7(6): 2087-2096.

Schmittgen T D, Livak K J. 2008. Analyzing real-time PCR data by the comparative CT method. Nature Protocols. 3(6): 1101-1108.

Tai M, Stephanopoulos G. 2013. Engineering the push and pull of lipid biosynthesis in oleaginous yeast Y. lipolytica for biofuel production. Metabolic engineering. 15: 1-9.

Thevenieau F, Le Dall M T, Nthangeni B, Mauersberger S, Marchal R, Nicaud J M. 2007. Characterization of Yarrowia lipolytica mutants affected in hydrophobic substrate utilization. Fungal Genet Biol. 44(6): 531-542.

Van de Velde S, Thevelein J M. 2008. Cyclic AMP-Protein Kinase A and Snf1 Signaling Mechanisms Underlie the Superior Potency of Sucrose for Induction of Filamentation in S. cerevisiae. Eucaryotic cell. 7(2): 286-293.

Walczak E, Robak M. 2009. Growth on sucrose of Y. lipolytica yeasts clones with invertase gene from S. cerevisiae [in Polish]. Acta Scientiarum Polonorum, Biotechnologia. 8 (4): 25-36.

Wang H J, Le Dall M T, Wach Y, Laroche C, Belin J M, Gaillardin C, Nicaud J M. 1999. Evaluation of acyl coenzyme A oxidase (Aox) isozyme function in the n-alkane-assimilating yeast Yarrowia lipolytica. Journal of Bacteriology 181(17): 5140-5148.

Wieczorke, R., S. Krampe, T. Weierstall, K. Freidel, C. P. Hollenberg and E. Boles 1999. Concurrent knock-out of at least 20 transporter genes is required to block uptake of hexoses in Sacccharomyces cervisiae. FEBS Letters 464(3): 123-128)

Wojtatowicz M, Rymowicz W. 1991. Effect of inoculum on kinetics and yield of citric acid production on glucose media by Y. lipolytica A-101. Acta Alimentaria Polonica. 41(2): 137-143.

Wojtatowicz M, Rymowicz W, Robak M, Żarowska B, Nicaud J M, 1997. Kinetics of cell growth and citric acid production by Y. lipolytica Suck transformants in sucrose media. Polish Journal of Food and Nutricion Sciences. 6/47 (4): 49-54.

Xuan J W, Fournier P, Declerck N, Chasles M, Gaillardin C. 1990. Overlapping reading frames at the LYS5 locus in the yeast Y. lipolytica. Molecular and Cellular Biology. 10: 4795-4806.

Yongmanitchai W, Ward O P, Growth of and omega-3 fatty acid production by Phaeodactylum tricornutum under different culture conditions. Appl. Environ. Microbiol. 57: 419-425, 1991

Young E, Poucher A, Comer A, Bailey A, Alper H S. 2011. Functional survey for heterologous sugar transport proteins, using Saccharomyces cerevisiae as a host. Appl Environ Microbiol 77(10): 3311-3319.

Young E M, Tong A, Bui H, Spofford C, Alper H S. 2014. Rewiring yeast sugar transporter preference through modifying a conserved protein motif. Proc Natl Acad Sci USA. 111(1): 131-136.

1. A Yarrowia lipolytica strain overexpressing a hexokinase gene, wherein said strain is of a background selected in the list consisting of A-101, H222 and W29, and said strain is capable of accumulating lipids.

2. The strain of claim 1, wherein the said hexokinase gene is ylHXK1.

3. The strain of claim 1, said strain further overexpressing a hexose transporter gene.

4. The strain of claim 1, wherein the said transporter is YHT1, YHT3 or YHT3-1181V.

5. The strain of claim 1, said strain further overexpressing the SUC2 gene of Saccharomyces cerevisiae.

6. The strain of claim 1, said strain further overexpressing the GPD1 gene and said strain being deficient for beta-oxidation of fatty acids.

7. The strain of claim 1, said strain further comprising at least one loss-of-function mutation in at least one gene selected from the PEX genes, the POX genes, the MFE1 gene, and the POT1 gene.

8. The strain of claim 1, said strain further comprising at least one loss-of-function mutation in each of the genes POX1, POX2, POX3, POX3, POX4, POX5, and POX6.

9. The strain of claim 1, said strain further comprising at least one additional mutation in at least one gene encoding an enzyme involved in the metabolism of fatty acids.

10. The strain of claim 9, wherein said mutation further increases the capacity of the strain to accumulate lipids.

11. The strain of claim 9, wherein said mutation is a mutation in GUT2, TLG3 or TLG4.

12. The strain of claim 9, wherein said mutation is a mutation in the YALI0B10153g.

13. The strain of claim 1, said strain further overexpressing the ylDGA2 gene.

14. A method for constructing the strain of claim 1 comprising a step of transforming an oleaginous yeast with a polynucleotide allowing the overexpression of said hexokinase gene.

15. The method of claim 14, further comprising at least one step of introducing at least one additional polynucleotide enabling the overexpression of another gene selected in the list comprising YHT1, YHT3, YHT3-I181V, SUC2, GPD1, and ylDGA2.

16. The method of claim 14, further comprising a step of introducing at least one additional mutation affecting lipid synthesis, wherein said mutation affects at least one of the PEX genes, one of the POX genes, the MFE1 gene, the POT1 gene, the TLG3 and TLG4 genes, GUT2, or YALI0B10153g.

17. A method for producing lipids, comprising the steps of:
a. growing the strain of oleaginous yeast of claim 1 in an appropriate culture medium; and
b. harvesting the lipids produced by the culture of step a.

18. The method of claim 17, wherein the culture medium of step a) comprises a carbon source which is glucose, fructose or sucrose.
